(19) United States
(12) Patent Application Publication
(10) Pub. No.: US 2024/0199521 A1
(43) Pub. Date: Jun. 20, 2024

(54) PREPARATION METHOD FOR TETRA-SUBSTITUTED ALLENOIC ACID COMPOUND BASED ON PALLADIUM CATALYTIC SYSTEM

(71) Applicant: FUDAN UNIVERSITY, Shanghai (CN)

(72) Inventors: Shengming MA, Shanghai (CN); Weifeng ZHENG, Shanghai (CN); Hui QIAN, Shanghai (CN)

(73) Assignee: FUDAN UNIVERSITY, Shanghai (CN)

(21) Appl. No.: 18/275,291

(22) PCT Filed: Jan. 19, 2022
(86) PCT No.: PCT/CN2022/074914

§ 371 Date: Aug. 1, 2023

(30) Foreign Application Priority Data
Feb. 2, 2021 (CN) 202110142014.7

(51) Int. Cl. C07C 51/12; B01J 31/02; B01J 31/22; B01J 31/24; C07C 59/52

(52) U.S. Cl. CPC C07C 51/12; B01J 31/0259; B01J 31/2282; B01J 31/2428; C07C 59/52; B01J 2531/004; B01J 2531/0205; B01J 2531/824

(57) ABSTRACT

Disclosed in the present invention is a preparation method for a tetra-substituted allenoic acid compound based on a palladium catalytic system, that is, a highly optically active allenoic acid compound having axial chirality is directly constructed in one step by reacting tertiary propargyl alcohol, carbon monoxide and water in an organic solvent under the action of a palladium catalyst, a chiral bisphosphine ligand, an organophosphoric acid, and an organic additive, and the theoretical yield can reach 100%. The method of the present invention is simple to operate, the raw materials and reagents are readily available, the reaction conditions are mild, the substrate universality is wide, the functional group compatibility is good, the reaction has high enantioselectivity (77%˜96% ee), and the reaction is well compatible with complex natural products or substrates of a drug molecular skeleton. The highly optically active allenoic acid compound obtained by the present invention can be used as an important intermediate for constructing a γ-butyrolactone compound containing a tetra-substituted chiral quaternary carbon center, tetra-substituted allenol, tetra-substituted allenal, tetra-substituted allenyl ketone, tetra-substituted allenami de and other compounds.

### TECHNICAL FIELD

The present invention belongs to the technical field of chemical synthesis, particularly to a method for directly synthesizing highly optically active tetra-substituted allenoic acid compounds.

### BACKGROUND OF THE INVENTION

Chiral allenes compounds are widely found in natural products, drug molecules and material science, and are a very important class of compounds (Ref: (a) Hoffmann-Röder, A.; Krause, N. Angew. Chem., Int. Ed. 2004, 43, 1196. (b) Rivera-Fuentes, P.; Diederich, F. Angew. Chem., Int. Ed. 2012, 51, 2818.). The axial chiral cumulative carbon-carbon double bonds in these compounds can be efficiently converted into central chiral compounds by one-step or multi-step reactions, which has important application value in synthetic chemistry. Therefore, how to efficiently construct chiral allenes compounds with high optically active has been widely concerned by synthetic chemists. How to construct tetra-substituted chiral quaternary carbon centers has been extensively studied in the past decade and has achieved fruitful results (Ref: (a) Quasdorf, K. W.; Overman, L. E. Nature 516, 2014, 181. (b) Zeng, X.-P.; Cao, Z.-Y.; Wang, Y.-H.; Zhou, F.; Zhou, J. Chem. Rev. 116, 2016, 7330.). Compared with the construction of compounds containing tetra-substituted chiral quaternary carbon centers, the synthesis of tetra-substituted axial chirality allenes compounds still faces great challenges. At present, the methods reported in the known literature for the synthesis of such compounds are still very limited, and can be generally divided into two categories: the asymmetric addition reaction of nucleophilic reagents to the conjugated alkyne system catalyzed by metal or organic small molecule and the stereoselectivity addition of allenyl group nucleophilic reagents to different electrophilic reagents. The main reason is that the accumulated carbon-carbon double bonds in the structure of chiral allenes are mutually perpendicular in space, and the substituents at 1,3-position of the allenes are located in a relatively far mutually perpendicular space. Compared with the compact spatial arrangement of central chirality, the formation of chiral allenes requires a larger chiral shielding environment in order to induce the formation of their axial chirality with high enantioselectivity, and excessive chiral shielding may lead to the decline of reaction activity. (Ref: (a) Hayashi, T.; Tokunaga, N.; Inoue, K. Org. Lett. 2004, 6, 305. (b) Qian, D.; Wu, L.; Lin, Z.; Sun, J. Nat. Commun. 2017, 8, 567. (c) Hashimoto, T.; Sakata, K.; Tamakuni, F.; Dutton, M. J.; Maruoka, K. Nat. Chem. 2013, 5, 240. (d) Mbofana, C. T.; Miller, S. J. J. Am. Chem. Soc. 2014, 136, 3285. (e) Zhang, P.; Huang, Q.; Cheng, Y.; Li, R.; Li, P.; Li, W. Org. Lett. 2019, 21, 503. (f) Zhang, L.; Han, Y.; Huang, A.; Zhang, P.; Li, P.; Li, W. Org. Lett. 2019, 21, 7415. (g) Chen, M.; Qian, D.; Sun, J. Org. Lett. 2019, 21, 8127. (h) Yang, J.; Wang, Z.; He, Z.; Li, G.; Hong, L.; Sun, W.; Wang, R. Angew. Chem., Int. Ed. 2020, 59, 642. (i) Li, X.; Sun, J. Angew. Chem., Int. Ed. 2020, 59, 17049. (j) Partridge, B. M.; Chausset-Boissarie, L.; Burns, M.; Pulis, A. P.; Aggarwal, V. K. Angew. Chem., Int. Ed. 2012, 51, 11795. Armstrong, R. J.; (k) Wu, S.; Huang, X.; Wu, W.; Li, P.; Fu, C.; Ma, S. Nat. Commun. 2015, 6, 7946. (1) Wang, G.; Liu, X.; Chen, Y.; Yang, J.; Li, J.; Lin, L.; Feng, X. ACS Catal. 2016, 6, 2482. (m) Tap, A.; Blond, A.; Wakchaure, V. N.; List, B. Angew. Chem., Int. Ed. 2016, 55, 8962. (n) Tang, Y.; Xu, J.; Yang, J.; Lin, L.; Feng, X.; Liu, X. Chem. 2018, 4, 1658. (o) Nandakumar, M.; Dias, R. M. P.; Noble, A.; Myers, E. L.; Aggarwal, V. K. Angew. Chem., Int. Ed. 2018, 57, 8203. (p) Liao, Y.; Yin, X.; Wnag, X.; Yu, W.; Fang, D.; Hu, L.; Wang, M.; Liao, J. Angew. Chem., Int. Ed. 2020, 59, 1176.).

Chiral allenoic acid compounds can be obtained by separated by the splitting method of racemic allenic acid compounds or allenic nitrile compounds (Ref: (a) Ma, S.; Wu, S. Chem. Commun. 2001, 0, 441. (b) Ao, Y.-F.; Wang, D.-X.; Zhao, L.; Wang, M.-X. J. Org. Chem. 2014, 79, 3103.) and the hydrolysis method of chiral allenoic acid esters (Ref: (a) Marshall, J. A.; Bartley, G. S.; Wallace, E. M. J. Org. Chem. 1996, 61, 5729. (b) Yu, J.; Chen, W.-J.; Gong, L.-Z. Org. Lett. 2010, 12, 4050), but the examples of the preparation of tetra-substituted allenoic acid compounds using the above methods are very limited. The above methods have the disadvantages of low reaction yield, narrow substrate range, poor functional group tolerance, and low atom economy and so on. Therefore, the development of a highly efficient and enantioselective method for the synthesis of tetra-substituted axial chirality allenoic acid compounds from simple and readily available raw materials will be an important breakthrough in the existing synthesis methods. In 2019, our research group used triphenylphosphine as the supporting ligand in the palladium/DTBM-SEGphos and phosphoric acid co catalytic system, and successfully prepared highly optically active chiral tetra-substituted allenoic acid compounds through the kinetic resolution of tertiary propargyl alcohol. This method has the advantages of a wide range of substrates and good functional group tolerance and mild reaction conditions, etc (Ref: Zheng, W.-F.; Zhang, W.; Huang, C.; Wu, P.; Qian, H.; Wang, L.; Guo, Y-L.; Ma, S. Nat. Catal. 2019, 2, 997.). On this basis, we successfully realized the high stereoselectivity and high yield (theoretical yield as high as 100%) of preparing tetra-substituted chiral allenoic acid compounds by means of dynamic kinetic chiral transfer of tertiary propargyl alcohol.

### SUMMARY OF THE INVENTION

The object of the present invention is to provide a method for directly synthesizing highly optically active axially chiral tetra-substituted allenoic acid compounds, that is, a one-step process for directly constructing high optically active axially chiral tetra-substituted allenoic acid compounds by using tertiary propargyl alcohol, carbon monoxide and water as reactants in organic additives and an organic solvent in the presence of palladium catalyst, chiral bisphosphine ligand and organophosphoric acid.

The object of the present invention is achieved by using the following solution:

The present invention provides a method for directly constructing highly optically active axially chiral tetra-substituted allenoic acid compounds includes: in the presence of palladium catalyst, chiral diphosphine ligand, and organophosphoric acid, the tertiary propargyl alcohol with different substituents, carbon monoxide and water undergo asymmetric allylation reaction in organic additives and organic solvent through transition metal catalysis, constructing highly optically active axially chiral tetra-substituted allenoic acid compounds in one-step synthesis. The reaction has the following reaction equation (a):

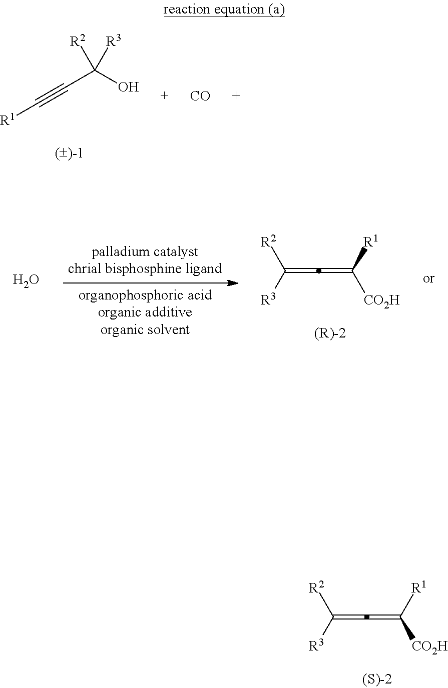

wherein,
R¹ is an alkyl, an alkyl with functional group, phenyl, aryl or heterocyclic group;
R² is an alkyl, an alkyl with functional group, phenyl, aryl or heterocyclic group;
R³ is an alkyl, an alkyl with functional group, phenyl, aryl or heterocyclic group;

In R¹, R² and R³, the functional group is selected from carbon-carbon triple bond, hydroxyl, acyl, acyloxy, amide, amino, and silicon group; said aryl group is phenyl group with electron-donating or electron-withdrawing substituents at the ortho, meta, and para positions; said heterocyclyl group is furyl or pyridyl group, or furan or pyridine with electron-donating or electron-withdrawing substituents.

Preferably,
R¹ is a C1-C30 alkyl, a C1-C30 alkyl with functional group at the end, phenyl, aryl or heterocyclic group;
R² is a C1-C10 alkyl, a C1-C10 alkyl with functional group at the end, phenyl, aryl or heterocyclic group;
R³ is a C1-C10 alkyl, a C1-C10 alkyl with functional group at the end, phenyl, aryl or heterocyclic group;

In R¹, R² and R³, the functional group is selected from carbon-carbon triple bond, hydroxyl, acyl, acyloxy, amide, amino, silicon group; said aryl group is phenyl group with electron-donating or electron-withdrawing substituents at the ortho, meta and para positions, said heterocyclic group is a furanyl or pyridyl group, or furan or pyridine with electron-donating or electron-withdrawing substituents; said electron-withdrawing substituents in the aryl or heterocyclic groups include halogen, nitro, ester, carboxyl, acyl, amide, and cyano groups, and the electron-donating substituents include alkyl, alkenyl, phenyl, alkoxy group, hydroxyl, amino, silicon group.

Further preferably,
R¹ is a C1-C20 alkyl, a C1-C20 alkyl with functional group at the end, phenyl, aryl or heterocyclic group;
R² is a C1-C10 alkyl, a C1-C10 alkyl with functional group at the end, phenyl, aryl or heterocyclic group;
R³ is a C1-C5 alkyl, a C1-C5 alkyl with functional group at the end, phenyl, aryl or heterocyclic group.

In R² and R³, the C1-C20 alkyl group is alkyl, alkenyl, phenyl, aryl or heteroaryl group; the C1-C10 alkyl group is alkyl, alkenyl, phenyl, aryl or heteroaryl group; the C1-C5 alkyl group is methyl, ethyl, n-propyl (and its isomers), n-butyl (and its isomers) and n-pentyl (and its isomers) group; in the C1-C20 alkyl group with functional group at the end or the C1-C10 alkyl group with functional group at the end or the C1-C5 alkyl group with functional group at the end, the functional group is selected from carbon-carbon triple bond, hydroxyl, acyl, acyloxy, amide, amino, silicon group; said aryl group is phenyl group with electron-donating or electron-withdrawing substituents at the ortho, meta and para positions, said heterocyclic group is a furanyl or pyridyl group, or furan or pyridine with electron-donating or electron-withdrawing substituents; said electron-withdrawing substituents in the aryl or heterocyclic groups include halogen, nitro, ester, carboxyl, acyl, amide, and cyano groups, and the electron-donating substituents include alkyl, alkenyl, phenyl, alkoxy group, hydroxyl, amino, silicon group.

Further preferably,
R¹ is selected from C1-C15 linear alkyl, C3-C15 cycloalkyl, C1-C15 alkyl with functional group at the end, phenyl, aryl or heterocyclic group;
R² is selected from C1-C10 linear alkyl, C3-C10 cycloalkyl, C1-C10 alkyl with functional group at the end, phenyl, aryl or heterocyclic group;
R³ is selected from C1-C5 linear alkyl, C3-C5 cycloalkyl, C1-C5 alkyl with functional group at the end, phenyl, aryl or heterocyclic group;

In R¹, R² and R³, in the C1-C15 alkyl group with functional group at the end or the C1-C10 alkyl group with functional group at the end or the C1-C5 alkyl group with functional group at the end, the functional group is selected from carbon-carbon triple bond, hydroxyl, acyl, acyloxy, amide, amino, silicon group; said aryl group is phenyl group with electron-donating or electron-withdrawing substituents at the ortho, meta and para positions, said heterocyclic group is a furanyl or pyridyl group, or furan or pyridine with electron-donating or electron-withdrawing substituents; said electron-withdrawing substituents in the aryl or heterocyclic groups include halogen, nitro, ester, carboxyl, acyl, amide, and cyano groups, and the electron-donating substituents include alkyl, alkenyl, phenyl, alkoxy group, hydroxyl, amino, silicon group.

Further preferably,
R¹ is selected from methyl, ethyl, n-propyl (and its isomers), n-butyl (and its isomers), n-pentyl (and its isomers), n-hexyl (and its isomers) body), n-heptyl (and its isomers), n-octyl (and its isomers), n-nonyl (and its isomers), n-decyl (and its isomers), n-undecyl (and its isomers), n-dodecyl (and its isomers), n-tridecyl (and its isomers), n-tetradecyl (and its isomers), n-pentadecyl (and its isomers), phenethyl, 4-chlorobutyl, 3-methylbutyl, 3-cyanopropyl, allyl, carbazolylpropyl, acetoxypropyl, silicon-protected propargyl, propargyl group;
R² is selected from n-propyl, cyclohexyl, tert-butyl, phenyl, o-methylphenyl, m-methylphenyl, p-methylphenyl, o-fluorophenyl, m-fluorophenyl, p-fluorophenyl, m-methoxyphenyl, p-i sopropylphenyl, p-chlorophenyl, p-bromophenyl, p-esterylphenyl, p-trifluoromethylphenyl, p-cyanophenyl, p-trimethylsilylphenyl, 2-naphthyl, 3-thienyl group;
R³ is selected from methyl, ethyl, propyl group.

As a further improvement, the present process comprises the following steps:
(1) adding a palladium catalyst, a chiral bisphosphine ligand and an organophosphoric acid in sequence into a dried reaction tube, plugging the reaction tube with a rubber stopper, connecting the vacuum pump, replacing with argon under argon atmosphere, adding a functionalized tertiary propargyl alcohol, water, and a certain volume of organic solvent and organic additives; freezing the reaction tube in liquid nitrogen bath, inserting carbon monoxide balloon, replacing with carbon monoxide into the reaction system under the atmosphere of carbon monoxide; after freezing and pumping, when the reaction system returns to the room temperature and melts, putting the reaction tube in the preset low-temperature bath or oil bath and stirring.

Wherein, the dosage of the organic solvent is 1.0-10.0 mL/mmol; preferably, is 5.0 mL/mmol. The dosage of functionalized tertiary propargyl alcohol (±1) shown in equation (a) is taken as the basis.
(2) after the completion of the reaction in step (1), raising the reaction tube from the low-temperature bath or oil bath, after returning to the room temperature, adding a certain volume of ethyl acetate into the reaction tube, filtering the resulting mixture with silica gel short column, washing with a certain amount of ethyl acetate, concentrating, and subjecting to the flash column chromatography, so as to obtain the highly optically active axially chiral allenoic acid compounds.

Wherein, the certain volume of the ethyl acetate refers to taking the amount of functionalized tertiary propargyl alcohol (±1) shown in equation (a) as basis, said amount of ethyl acetate is 1.0-100 mL/mmol; preferably, is 5.0 mL/mmol.

As a further improvement, the palladium catalyst used in the present invention is any one or more of dis-(allyl-palladium chloride), tetra-(triphenylphosphine)palladium, tri-(dibenzylidene-acetone)dipalladium, dis-(cinnamyl-palladium chloride), dis-(dibenzylidene-acetone)monopalladium, palladium chloride, palladium acetate, dis-(triphenylphosphine)palladium chloride, bis-(acetonitrile)palladium chloride, and so on; preferably, is dis-(allyl-palladium chloride).

As a further improvement, the chiral diphosphine ligand used in the present invention is selected from one or more of (R)-L1˜(R)-L4 and its enantiomers (S)-L1˜(S)-L4 in the following structure; preferably, the chiral diphosphine ligand is (R)-L4 and/or its enantiomer (S)-L4.

Wherein, Ar is a phenyl, an aryl or heterocyclic group; said aryl group is a phenyl group substituted by alkyl or alkoxy group at the ortho, meta, and para positions; wherein said alkyl group includes methyl, trifluoromethyl, ethyl, propyl, isopropyl, butyl, isobutyl, tert-butyl group; said alkoxy group includes methoxy, ethoxyl, propoxyl, isopropoxyl, butoxyl, isobutyloxyl, tert butoxyl group; said heterocyclic group is thienyl, furyl or pyridyl group; preferably, said Ar is phenyl, 4-methylphenyl, 3,5-dimethylphenyl, 3,5-ditrifluoromethylphenyl, 3,5-dimethyl-4-methoxyphenyl, 3,5-ditert-butyl-4-methoxyphenyl group.

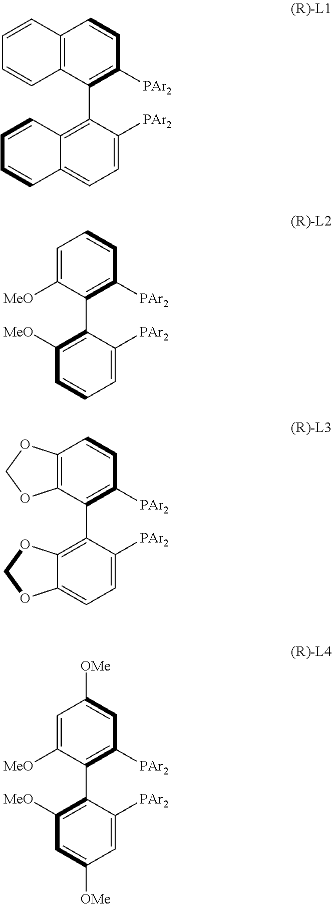

As a further improvement, the chiral diphosphine ligand used in the present invention is selected from one or more of (R)-L4a, (R)-L4b, (R)-L4c, (R)-L4d, (R)-L4e, (R)-L4f and its enantiomer (S)-L4a, (S)-L4b, (S)-L4c, (S)-L4d, (S)-L4e, (S)-L4f; wherein, the structure of said (R)-L4a, (R)-L4b, (R)-L4c, (R)-L4d, (R)-L4e, (R)-L4f is as follows:

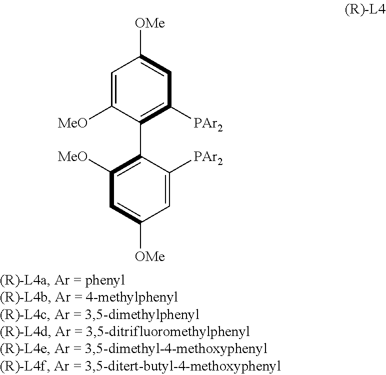

As a further improvement, the organophosphoric acid used in the present invention is selected from any one or more of organophosphoric acid 1, organophosphoric acid 2, organophosphoric acid 3, and so on; wherein, R⁴ is hydrogen, C1˜C6 alkyl, phenyl or aryl group; said aryl group is a phenyl group substituted by C1˜C6 alky at the ortho, meta, and para positions; R⁵ is hydrogen, R⁵ is C1˜C6 alkyl, phenyl or aryl group, and said aryl group is a phenyl group substituted by C1-C6 alky, halogenated alkyl, alkoxy group, halogens, nitro group at the ortho, meta, and para positions; preferably, R⁴ is phenyl group, R⁵ is 3,5-ditrifluoromethylphenyl group.

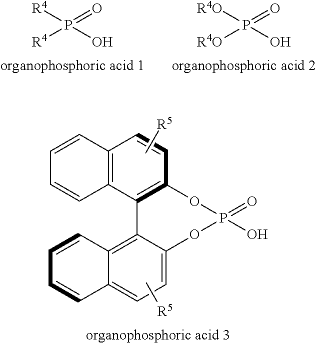

As a further improvement, the organic solvent is selected from any one or more of N-methyl pyrrolidone, 1,4-dioxane, tetrahydrofuran, acetonitrile, methyl tert-butyl ether, fluorobenzene, chlorobenzene, bromobenzene, iodobenzene, toluene, 1,2-xylene, 1,3-xylene, 1,4-xylene, mesitylene, 4-ethyltoluene, 1,4-diethylbenzene, triethylbenzene, trifluorotoluene, dichloromethane, dibromomethane, 1,1-dichloroethane, 1,2-dichloroethane, 1,2-dibromoethane, chloroform, acetic acid, N,N-dimethylformamide and dimethyl sulfoxide, and so on; preferably, is toluene.

As a further improvement, the organic additive is selected from any one or more of 1,1-bis(diphenylphosphine)methane, 1,2-bis(diphenylphosphine)ethane, 1,3-bis(diphenylphosphine)propane, 1,4-bis(diphenylphosphine)butane, 1,1′-bis (diphenylphosphine)ferrocene, bis(2-diphenylphosphine)ether, 4,5-bis(diphenylphosphino)-9,9-dimethylxanthene, 1,1′-binaphthyl-2,2′-bisdiphenylphosphine, triphenylphosphine, tri(4-methoxyphenyl)phosphine, tri(4-methylphenyl)phosphine, tri(4-fluorophenyl)phosphine, tris(4-trifluoromethylphenyl)phosphine, dichloromethane, dibromomethane, chloroform, bromoform, carbon tetrachloride, bromoethane, bromobutane, benzene, fluorobenzene, 1,4-difluorobenzene, hexafluorobenzene, chlorobenzene, 1,4-dichlorobenzene, bromobenzene, 1,4-dibromobenzene, 4-methoxyromobenzene, 4-methylbromobenzene, 4-fluorobromobenzene, 4-trifluoromethylbromobenzene, iodobenzene, trifluorotoluene, aniline, benzenesulfonic acid, phenol, phenylboronic acid, and so on; preferably, is bromobenzene.

As a further improvement, said reaction temperature of the present invention is −20˜100° C.; preferably, is 0˜80° C.; more preferably, is 25˜70° C.

As a further improvement, said reaction time of the present invention is 1-36 hours; preferably, is 12 hours.

As a further improvement, said molar ratio of tertiary propargyl alcohol (±1) with different substituents, water, palladium catalyst, chiral diphosphine ligand, organophosphoric acid and organic additives of the present invention is 1.0:(1.0-30.0):(0.005-0.1):(0.005-0.1):(0.01-0.3):(1.0-30.0); preferably, is 1.0:20.0:0.04:0.06:0.025:10.0.

Under the heating conditions of the synthetic method of the present invention, the following four technical difficulties are mainly overcome, as shown in the following reaction equation (b):

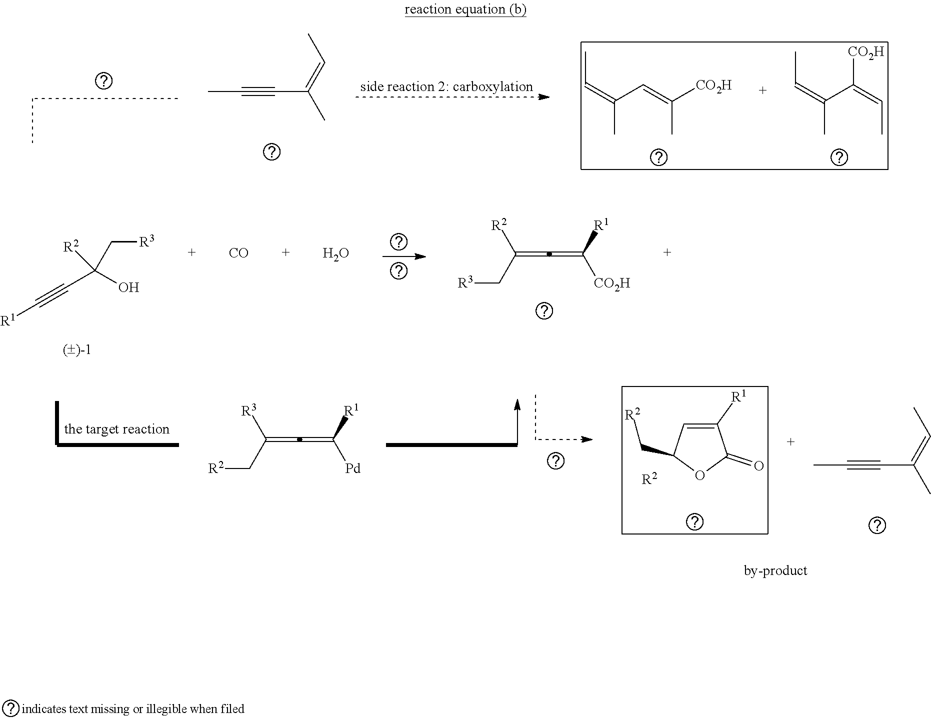

1) In the presence of phosphoric acid, the propargyl alcohol raw material is prone to side reaction elimination reaction due to heating, and generates by-product enyne, which causes the target reaction to fail to occur smoothly (side reaction 1);
2) The by-product enyne palladium catalytic system is prone to carboxylation in the presence of carbon monoxide and water to generate two conjugated dienoic acid isomer by-products with physical properties very similar to chiral allenoic acid, resulting in the inability to separate and purify the target product of the reaction, which affects the practicality of the reaction (side reaction 2);
3) The chiral allenoic acid product is unstable, and in the presence of a transition metal catalyst, prone to further lactone cycloisomerization into γ-butyrolactone by-products, resulting in a decrease in the yield of chiral allenoic acid (side reaction 3).
4) Under heating conditions, the chiral allenoic acid product will coordinate with the transition metal catalyst and undergo partial racemization, resulting in a decrease in the ee value.

The present invention can effectively overcome the above technical difficulties by using organic additives (such as bromobenzene and bromobenzene derivatives that donate electrons or withdraw electrons on the benzene ring), and successfully realize the preparation of chiral allenoic acid compounds with high enantioselectivity , and avoid the formation of other by-products in the reaction process, and exclusively obtain chiral allenoic acid compounds. Only in the process of condition optimization, (E)-conjugated dienoic acid 1, (E)-conjugated dienoic acid 2 are observed, under optimal conditions, only a small amount of enyne, γ-butyrolactone by-product can be observed in some reactions.

The present invention proposes the following possible mechanisms for the reaction described in the present invention:
(1) The palladium catalyst [Pd(π-allyl)Cl]₂ is simultaneously coordinated with chiral ligands (R)- or (S)-BTFM-Garphos and bromobenzene, and then in situ reduction generates catalytically active zero-valent palladium species I, and palladium species I is characterized by the possibility of coordinating both chiral bisphosphine ligands and bromine atoms in bromobenzene.
(2) Palladium-catalyst I and two different configurations of tertiary propargyl alcohols II or III activated by chiral phosphate CPA undergo S_{N}2′ oxidative addition to generate a pair of allenyl palladium diastereomers IV and V, because the reaction rates of allenyl palladium intermediates IV and V with carbon monoxide and water are quite different, and the reaction rate of allenyl palladium intermediate IV is much higher than that of allenyl palladium intermediate V (K_{VI}>>K_{V}), and the allenyl palladium intermediate V can undergo dynamic kinetic chirality transfer, is gradually converted into the allenyl palladium intermediate IV, and the subsequent reaction occurs with a single allenyl palladium intermediate.
(3) After the allenyl palladium intermediate IV reacts with carbon monoxide by the carbon monoxide intercalation, it accepts the nucleophilic attack of water to form the carbonyl-substituted allenyl palladium intermediate VI or VII.
(4) The chiral allenoic acid product is obtained after the reduction and elimination of the carbonyl-substituted allenyl palladium intermediate VI or VII, and the catalytic zero-valent palladium species I is released simultaneously, and the zero-valent palladium species I will participate in a new catalytic cycle again. The specific mechanism is shown in the following formula (c).

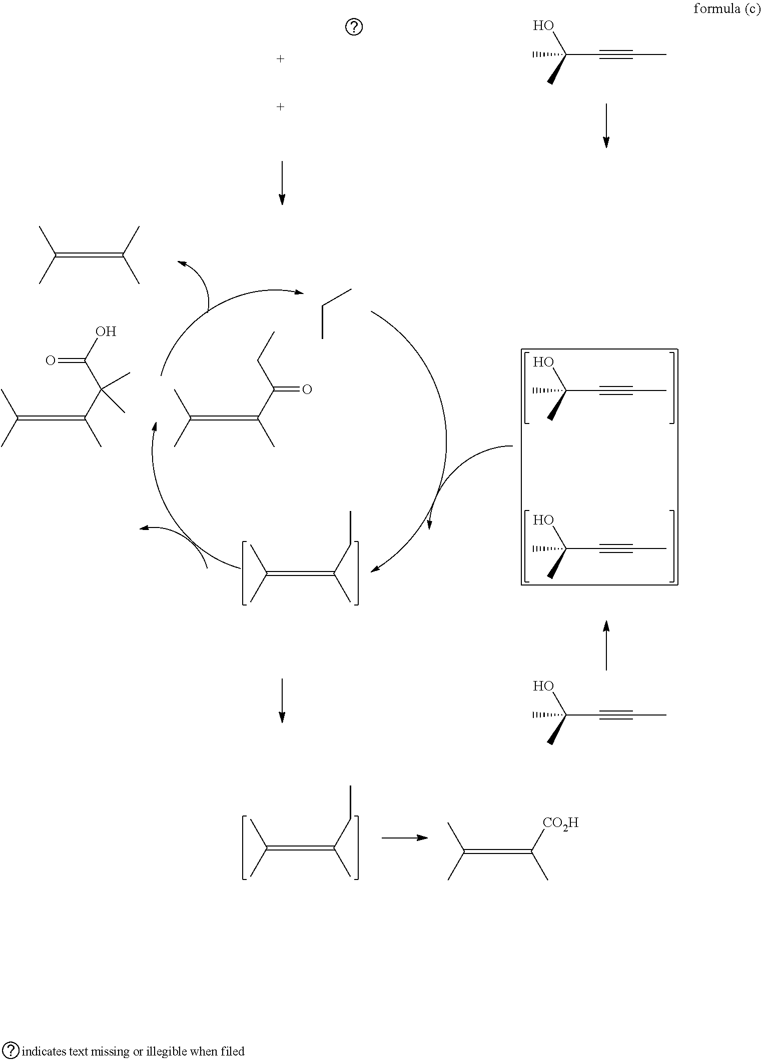

The present invention also provides highly optically active axially chiral allenoic acid compounds, the structure of which is shown as(R)-2, (S)-2:

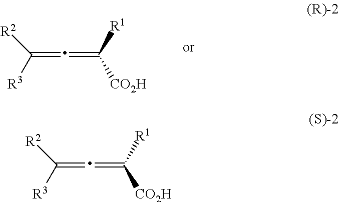

Wherein,

The definitions of R¹, R² and R³ are the same as those of the reaction equation (a).

The list of newly prepared compounds in the synthesis process of the present invention is shown in Table 1 below:

TABLE 1

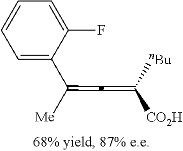

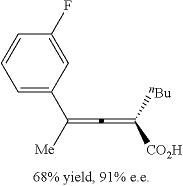

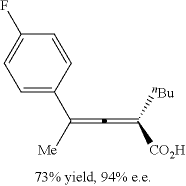

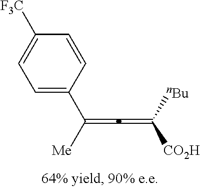

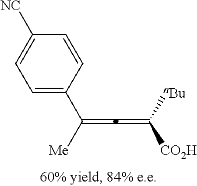

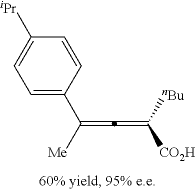

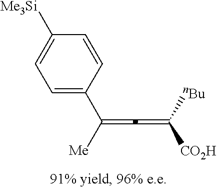

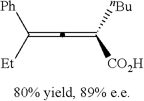

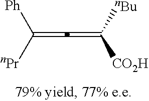

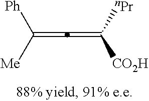

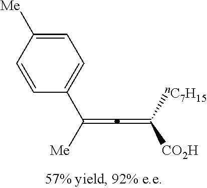

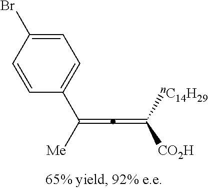

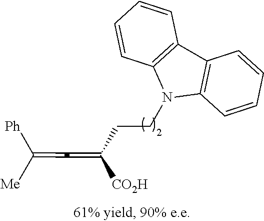

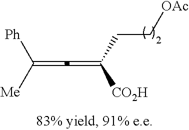

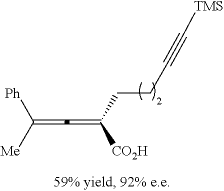

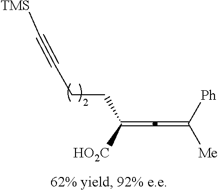

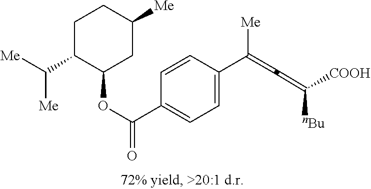

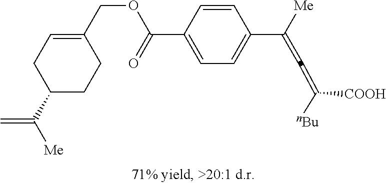

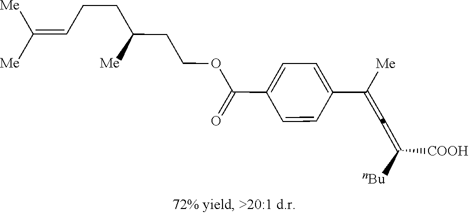

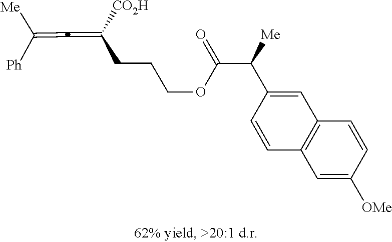

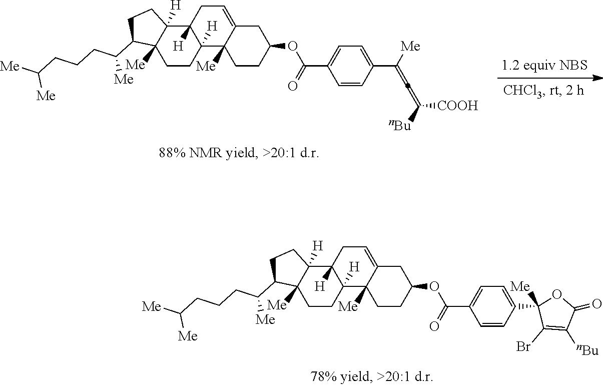

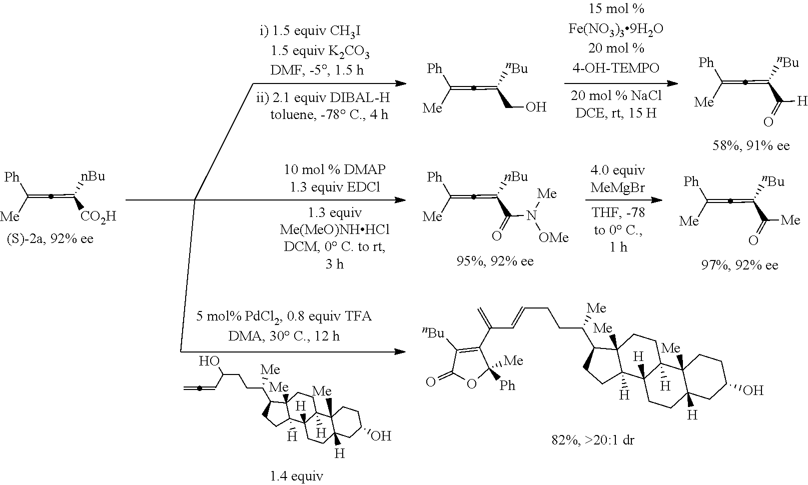

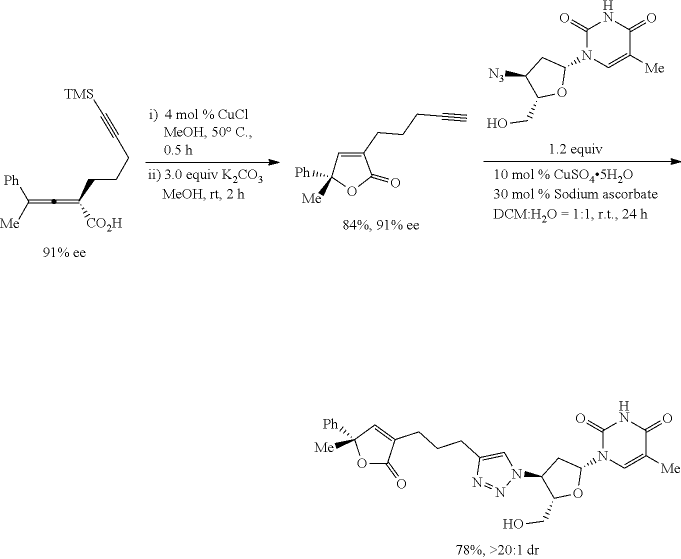

The present invention also provides the application of the highly optically active axially chiral allenoic acid compound shown in formula (R)-2 in the preparation of γ-butyrolactone compounds containing tetrasubstituted chiral quaternary carbon centers, tetrasubstituted allenic alcohol, tetra-substituted allenal, tetra-substituted allenyl ketone, tetra-substituted allenamide and other compounds.

The comparison list of the method of the present invention and the original method:

TABLE 2

| Type | Prior art: | Technical solution of the present invention: |
|---|---|---|
| 1. Different | PdCl₂ | [Pd(π-allyl)Cl]₂ |
| types of | | |
| catalysts: | | |
| 2. Different chiral ligands: | 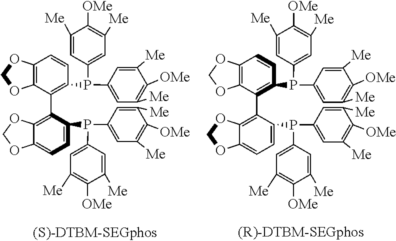 | 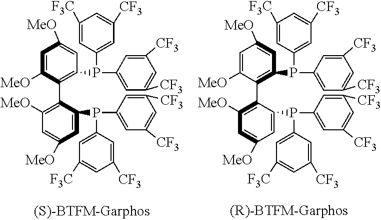 |
| 3. Different organo- phosphoric acids | 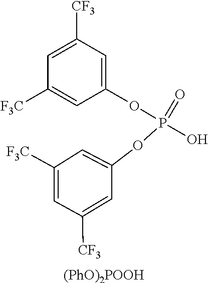 [SMILES: "(PhO)2POOH" ; O=P(O)(Oc1cc(C(F)(F)F)cc(C(F)(F)F)c1)Oc1cc(C(F)(F)F)cc(C(F)(F)F)c1] | 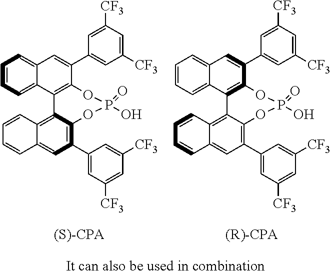 |
| 4. Different additive | 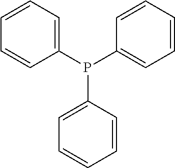 [SMILES: c1ccc(P(c2ccccc2)c2ccccc2)cc1] | 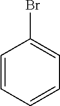 |
| 5. Different | -5° C. or 0° C. | 50° C. or 65° C. |
| reaction | | |
| temperature | | |
| 6. Different | 18 hours to 72 hours | 3 to 24 hours |
| reaction | | |
| time | | |
| 7. Different | Kinetic resolution process | Dynamic kinetic chiral transfer |
| reaction | | |
| concept | | |
| 8. Different | The enantioselectivity of the reaction is enhanced by | The enantioselectivity of the reaction is enhanced by bromo- |
| reaction | triphenyl phosphine as an auxiliary ligand, but only | benzene as a transient coordination ligand, which promotes |
| mechanism | one enantiomer of the raw material can react | the isomerizationof the key intermediates of the reaction and |
| | | ensures that the product does not undergo racemization |
| | | lactone cycloisomerization |
| 9. Different | Theoretically up to 50% | Theoretically up to 100% |
| total | | |
| reaction | | |
| yield | | |

The innovation of the present invention include:
(1) The reaction described in the present invention starts from the simple and readily available tertiary propargyl alcohol, and under the co-catalysis system of palladium and phosphoric acid, through the dynamic kinetic chirality transfer process, successfully achieves the highly enantioselective preparation of chiral tetra-substituted allenoic acid compounds. The theoretical yield of this reaction can reach 100%, while the prior art is kinetic resolution reaction, and the highest theoretical yield is 50% (see Table 2).
(2) bromobenzene is usually used as an electrophile in the coupling reaction, and in the reaction of the present invention, in the form of a transient coordination ligand, interacts with palladium and participates in the reaction catalysis cycle, while its bromobenzene itself does not participate in the reaction. Due to the use of bromobenzene as the additive, the present invention successfully overcomes or breaks through the technical barriers and technical limitations existing in the original kinetic resolution method, that is, the isomerization conversion of two key allenyl palladium intermediates cannot be realized, and promotes the dynamic kinetic chiral transfer of the allenyl palladium intermediates V with slow reaction rate to the allenyl palladium intermediates IV with fast reaction rate, which can undergo the dynamic kinetic chiral transfer and increase the reaction yield to 100%.
(3) After bromobenzene is coordinated with palladium as a transient coordination ligand in the reaction of the present invention, it can play the role of accelerating the isomerization of the allenyl palladium intermediate V with slow reaction rate to the allenyl palladium intermediate IV with fast reaction rate, which not only significantly improves the enantioselectivity of the reaction, but also accelerates the target conversion to a certain extent to achieve the effect of improving the reaction yield.
(4) bromobenzene can effectively inhibit the chiral allenoic acid product from coordinating with palladium after coordinating with palladium as a transient coordination ligand in the reaction of the present invention, thereby preventing the product from being racemized or further lactone cycloisomerization to form lactones compounds.
(5) In particular, the consumption of bromobenzene is at least ten times the equivalent of propargyl alcohol, otherwise the enantioselectivity of the reaction will decrease significantly.

The present invention has the following advantages: the present invention uses simple and readily available functionalized tertiary propargyl alcohol as starting material, under the action of palladium catalyst, chiral bisphosphine ligand, organophosphoric acid, organic additive and an organic solvent, for the first time, achieves the one-step synthesis of highly optically active axially chiral tetra-substituted allenoic acids compounds by dynamic kinetic chirality transfer. The chiral allenoic acid compounds obtained in the present invention can be used as important synthetic intermediates for constructing γ-butyrolactone compounds containing tetra-substituted chiral quaternary carbon centers, or converted into, tetra-substituted allenic alcohol, tetra-substituted allenal, tetra-substituted allenyl ketone, tetra-substituted allenamide and other compounds. The raw materials and reagents are readily available, preparation is convenient; the reaction conditions are mild, operations are simple; the substrate is widely applicable; the functional group compatibility is good; highly optically axially chirality pure tetrasubstituted allenoic acid compounds can be synthesized in one step; the product has high enantioselectivity (77% ee˜96% ee); the reaction can be applied to the later modification of complex molecules containing natural product backbones or drug molecule fragments; the product is easy to separate and purify; the product can be converted in one or more steps into different functional groups substituted tetra-substituted chiral allenes compounds or γ-butyrolactones compounds containing a chiral quaternary carbon center, etc.

### PREFERRED EMBODIMENTS OF THE INVENTION

The following examples are given to further illustrating the specific solutions of the present invention. The process, conditions, experimental methods, and so on for implementing the present invention are all general knowledge and common knowledge in the field except for the contents specifically mentioned below, and the present invention has no special limitation. The specific structural formula and the corresponding number (including their enantiomers) of chiral diphosphine ligands involved in all the examples are as follows:

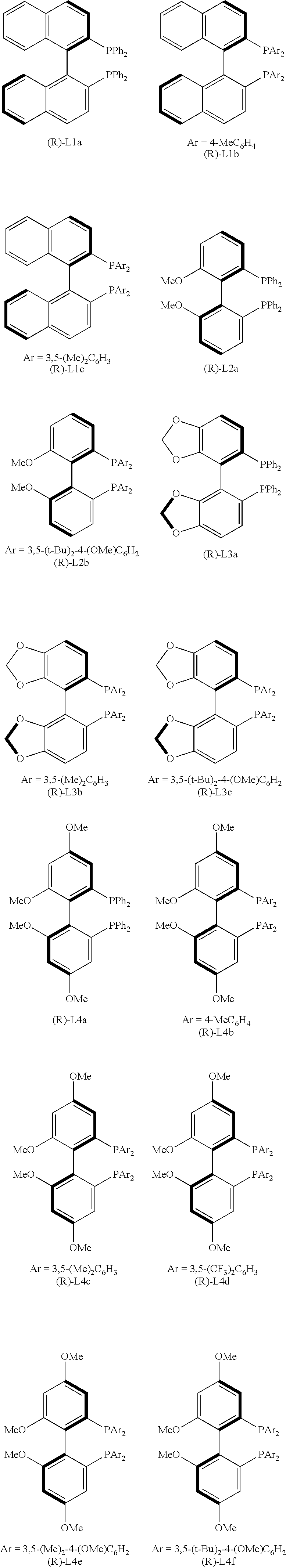

The specific structural formula and the corresponding number (including their enantiomers) of organophosphoric acid involved in all the examples are as follows:

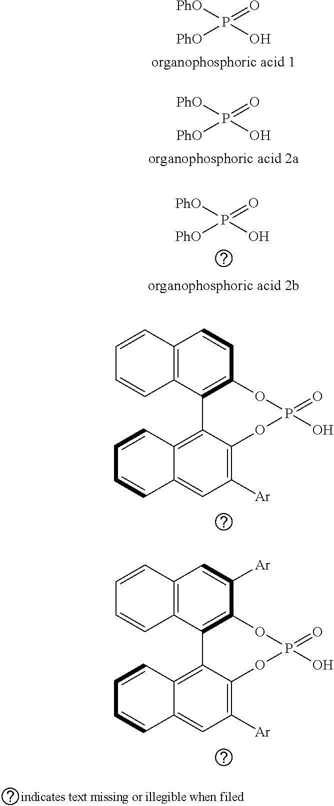

### EXAMPLE 1

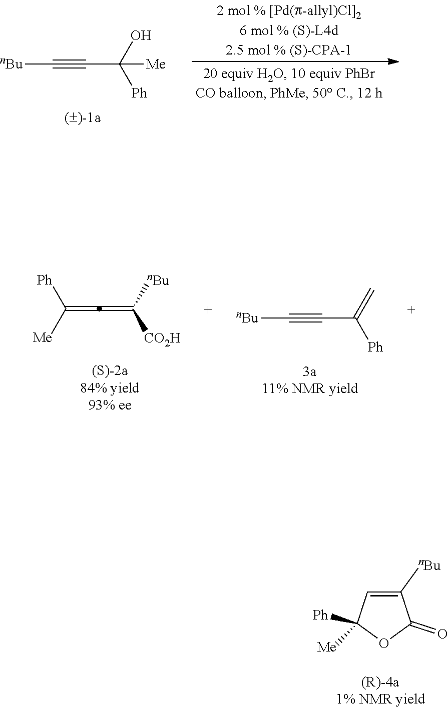

Wherein, “mol” refers to mole, “PhBr” refers to bromobenzene, “PhMe” refers to toluene, “CO balloon” refers to carbon monoxide balloon, “ee” refers to the percentage of enantiomeric excess.

[Pd(π-allyl)Cl]₂ (0.0015 g, 0.004 mmol), chiral bisphosphine ligand (S)-L4d (0.0148 g, 0.012 mmol), (S)-CPA-1 (0.0039 g, 0.005 mmol) were added in sequence to a dry Schlenk reaction tube. The reaction tube was plugged with a rubber stopper, and then connected with the vacuum pump, and replaced with the argon three times under an argon atmosphere. And under the protection of the argon, tertiary propargyl alcohol (±)-1a (0.0402 g, 0.2 mmol), toluene (0.8 mL), bromobenzene (211 μL, d=1.49 g/mL, 0.3144 g, 2 mmol) and water (72 μL, d=1.0 g/mL, 0.072 g, 4 mmol) were added. After the argon was closed, the reaction tube was placed in a liquid nitrogen bath to freeze for 3 minutes, inserted by carbon monoxide balloon (about 1 liter), replaced with carbon monoxide three times under a carbon monoxide atmosphere, then the liquid nitrogen bath was removed. After the reaction system returned to the room temperature and melted into liquid, the reaction tube was placed in an oil bath that had been preheated to 50° C. and stirred for 12 hours. The reaction was taken out of the oil bath, and after returning to room temperature, added with H₂O₂ (8 μL, d=1.13 g/mL, 30 wt. % in H₂O, 0.0027 g, 0.08 mmol). After stirred at room temperature for 30 minutes, the reaction solution was diluted by adding ethyl acetate (1 mL), and the resulting mixture was filtered through a short silica gel column (1 cm), washed with ethyl acetate (5 mL), concentrated, and subjected to flash column chromatography (eluent: petroleum ether (60˜90° C.)/ethyl acetate=15/1, then 10/1) to to afford a product: chiral allenoic acid (S)-2a (0.0385 g, 84%): solid; 93% ee (HPLC conditions: AS-H column, hexane/i-PrOH=98/2, 1.0 mL/min, λ=214 nm, t_{R} (major)=8.7 min, t_{R}(minor)=12.1 min); ¹H NMR (400 MHz, CDCl₃): δ=7.44-7.27 (m, 4 H, Ar—H), 7.27-7.21 (m, 1 H, Ar—H), 2.32 (t, J=7.6 Hz, 2 H, CH₂), 2.19 (s, 3 H, CH₃), 1.52-1.40 (m, 2 H, CH₂), 1.40-1.29 (m, 2 H, CH₂), 0.88 (t, J=7.2 Hz, 3 H, CH₃); ¹³C NMR (100 MHz, CDCl₃): δ=212.6, 172.8, 135.0, 128.5, 127.6, 126.1, 105.2, 101.8, 30.2, 28.3, 22.2, 16.3, 13.8.

### EXAMPLE 2

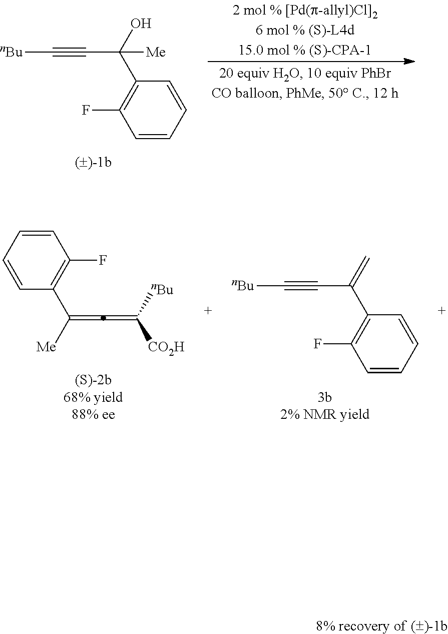

Operations were conducted by referring to Example 1. [Pd(π-allyl)Cl]₂ (0.0037 g, 0.01 mmol), chiral bisphosphine ligand (S)-L4d (0.0366 g, 0.03 mmol), (S)-CPA-1 (0.0601 g, 0.075 mmol), (±)-1b (0.1104 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 10/1) to afford a product: chiral allenoic acid (S)-2b (0.0841 g, 68%): oil; 88% ee (HPLC conditions: AS-H column, hexane/ⁱPrOH=98/2, 1.0 mL/min, λ=214 nm, t_{R}(major)=9.5 min, t_{R}(minor)=13.0 min); [α]_{D}²⁷=+37.5 (c=1.06, CHCl₃); ¹H NMR (400 MHz, CDCl₃): δ=7.33 (td, J₁=7.8 Hz, J₂=1.7 Hz, 1 H, Ar—H), 7.27-7.21 (m, 1 H, Ar—H), 7.12 (td, J₁=7.5 Hz, J₂=1.1 Hz, 1 H, Ar—H), 7.07-7.00 (m, 1 H, Ar—H), 2.36-2.24 (m, 2 H, CH₂), 2.24-2.13 (m, 3 H, CH₃), 1.53-1.41 (m, 2 H, CH₂), 1.39-1.27 (m, 2 H, CH₂), 0.89 (t, J=7.2 Hz, 2 H, CH₂); ¹³C NMR (100 MHz, CDCl₃): δ=212.9 (d, J=1.6 Hz), 173.1, 160.3 (d, J=248.8 Hz), 129.1 (d, J=8.7 Hz), 128.9 (d, J=3.1 Hz), 124.1 (d, J=3.2 Hz), 123.6 (d, J=11.9 Hz), 116.0 (d, J=22.1 Hz), 100.4, 99.9 (d, J=1.6 Hz), 30.0, 28.2, 22.2, 17.9 (d, J=2.4 Hz), 13.8; ¹⁹F NMR (376 MHz, CDCl₃): δ=−112.1; IR (neat): ν=2957, 2929, 2859, 1943, 1681, 1493, 1279, 1079 cm⁻¹; MS (70 eV, EI) m/z (%): 248 (M⁺, 2.21), 161 (100); HRMS calcd for C₁₅H₁₇FO₂ [M⁺]: 248.1207, found: 248.1207.

### EXAMPLE 3

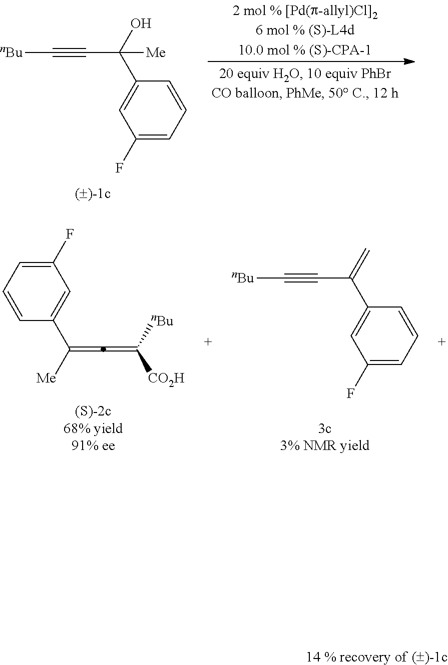

Operations were conducted by referring to Example 1. [Pd(π-allyl)Cl]₂ (0.0037 g, 0.01 mmol), chiral bisphosphine ligand (S)-L4d (0.0367 g, 0.03 mmol), (S)-CPA-1 (0.0402 g, 0.05 mmol), (±)-1c (0.1104 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 10/1) to afford a product: chiral allenoic acid (S)-2c (0.0847 g, 68%): white solid; 91% ee (HPLC conditions: AS-H column, hexane/ⁱPrOH=98/2, 1.0 mL/min, λ=214 nm, t_{R}(major)=8.0 min, t_{R}(minor)=11.8 min); [α]_{D}²⁷=+18.0 (c=1.00, CHCl₃); melting point: 104.1-105.2° C. (petroleum ether/DCM); ¹H NMR (400 MHz, CDCl₃): δ=7.34-7.23 (m, 1 H, Ar—H), 7.16 (d, J=8.0 Hz, 1 H, Ar—H), 7.07 (dt, J₁=10.4 Hz, J₂=2.0 Hz, 1 H, Ar—H), 6.94 (td, J₁=7.9 Hz, J₂=2.3 Hz, 1 H, Ar—H), 2.33 (t, J=7.4 Hz, 2 H, CH₂), 2.17 (s, 3 H, CH₃), 1.51-1.41 (m, 2 H, CH₂), 1.41-1.30 (m, 2 H, CH₂), 0.88 (t, J=7.4 Hz, 2 H, CH₂); ¹³C NMR (100 MHz, CDCl₃): δ=212.6, 172.7, 163.1 (d, J=244.1 Hz), 137.5 (d, J=7.1 Hz), 129.9 (d, J=8.6 Hz), 121.7 (d, J=2.4 Hz), 114.4 (d, J=21.3 Hz), 112.9 (d, J=22.9 Hz), 104.5 (d, J=3.1 Hz), 102.3, 30.1, 28.2, 22.2, 16.2, 13.8; ¹⁹F NMR (376 MHz, CDCl₃): δ=−113.6; IR (neat): ν=2961, 2929, 2863, 1937, 1685, 1422, 1264, 1089, 1021 cm⁻¹; MS (70 eV, EI) m/z (%): 248 (M⁺, 3.61), 161 (100); Anal. Calcd. for C₁₅H₁₇FO₂: C 72.56, H 6.90; found C 72.50, H 7.14.

### EXAMPLE 4

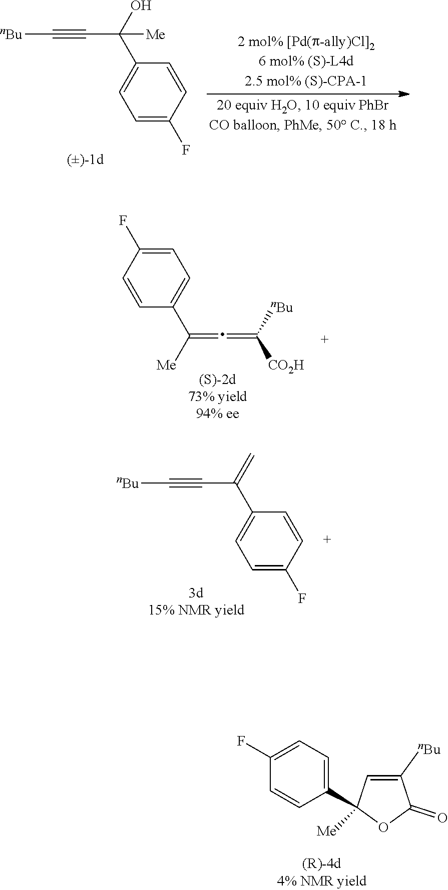

Operations were conducted by referring to Example 1. [Pd(π-allyl)Cl]₂ (0.0036 g, 0.01 mmol), chiral bisphosphine ligand (S)-L4d (0.0368 g, 0.03 mmol), (S)-CPA-1 (0.0101 g, 0.0125 mmol), (±)-1d (0.1104 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1, then 10/1) to afford a product: chiral allenoic acid (S)-2d (0.0911 g, 73%): white solid; 94% ee (HPLC conditions: AS-H column, hexane/ⁱPrOH=98/2, 1.0 mL/min, λ=214 nm, t_{R}(major)=8.9 min, t_{R}(minor)=11.9 min); [α]_{D}²⁸=+18.7 (c=1.00, CHCl₃); melting point: 113.0-114.0° C. (petroleum ether/DCM); ¹H NMR (400 MHz, CDCl₃): δ=7.41-7.29 (m, 2 H, Ar—H), 7.09-6.96 (m, 2 H, Ar—H), 2.32 (t, J=7.6 Hz, 2 H, CH₂), 2.17 (s, 3 H, CH₃), 1.51-1.40 (m, 2 H, CH₂), 1.40-1.29 (m, 2 H, CH₂), 0.88 (t, J=7.4 Hz, 3 H, CH₃); ¹³C NMR (100 MHz, CDCl₃): δ=212.3 (d, J=2.4 Hz), 172.8, 162.3 (d, J=245.7 Hz), 131.0 (d, J=3.2 Hz), 127.7 (d, J=8.7 Hz), 115.5 (d, J=21.3 Hz), 104.4, 101.9, 30.2, 28.3, 22.2, 16.5, 13.8; ¹⁹F NMR (376 MHz, CDCl₃): δ=−115.0; IR (neat): ν=2940, 2868, 1939, 1683, 1507, 1284, 1233 cm⁻¹; MS (70 eV, EI) m/z (%): 248 (M⁺, 2.68), 161 (100); Anal. Calcd. for C₁₅H₁₇FO₂: C 72.56, H 6.90; found C 72.72, H 7.14.

### EXAMPLE 5

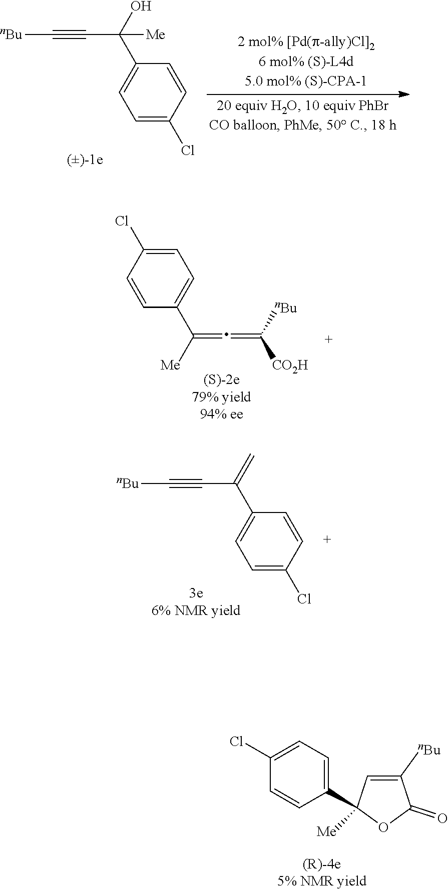

Operations were conducted by referring to Example 1 [Pd(π-allyl)Cl]₂ (0.0016 g, 0.004 mmol), chiral bisphosphine ligand (S)-L4d (0.0149 g, 0.0012 mmol), (S)-CPA-1 (0.0081 g, 0.01 mmol), (±)-1e (0.0471 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2e (0.0415 g, 79%): white solid; 93% ee (HPLC conditions: AS-H column, hexane/ⁱPrOH=98/2, 1.0 mL/min, λ=214 nm, t_{R}(major)=9.7 min, t_{R}(minor)=13.3 min); ¹H NMR (400 MHz, CDCl₃): δ=7.30 (s, 4 H, Ar—H), 2.32 (t, J=7.4 Hz, 2 H, CH₂), 2.17 (s, 3 H, CH₃), 1.49-1.40 (m, 2 H, CH₂), 1.40-1.29 (m, 2 H, CH₂), 0.88 (t, J=7.2 Hz, 3 H, CH₃); ¹³C NMR (100 MHz, CDCl₃): δ=212.4, 172.3, 133.6, 133.4, 128.7, 127.3, 104.4, 102.1, 30.2, 28.3, 22.2, 16.3, 13.8.

### EXAMPLE 6

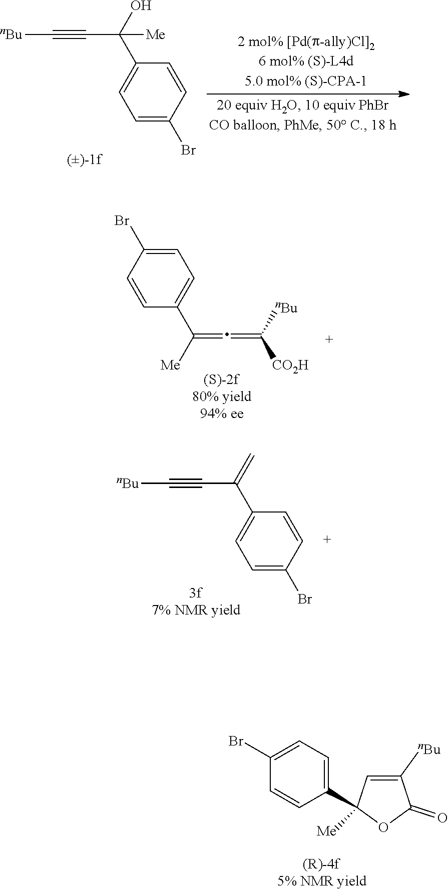

Operations were conducted by referring to Example 1. [Pd(π-allyl)Cl]₂ (0.0016 g, 0.004 mmol), chiral bisphosphine ligand (S)-L4d (0.0149 g, 0.0012 mmol), (S)-CPA-1 (0.008 g, 0.01 mmol), (±)-1f (0.0565 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2f (0.0499 g, 80%): white solid; 94% ee (HPLC conditions: AS-H column, hexane/ⁱPrOH=98/2, 1.0 mL/min, λ=214 nm, t_{R}(major)=10.5 min, t_{R}(minor)=14.8 min); ¹H NMR (400 MHz, CDCl₃): δ=7.50-7.41 (m, 2 H, Ar—H), 7.26-7.19 (m, 2 H, Ar—H), 2.32 (d, J=7.4 Hz, 2 H, CH₂), 2.16 (s, 3 H, CH₃), 1.49-1.39 (m, 2 H, CH₂), 1.39-1.29 (m, 2 H, CH₂), 0.88 (t, J=7.2 Hz, 3 H, CH₃); ¹³C NMR (100 MHz, CDCl₃): δ=212.4, 172.4, 134.1, 131.7, 127.6, 121.5, 104.5, 102.2, 30.2, 28.2, 22.2, 16.2, 13.8.

### EXAMPLE 7

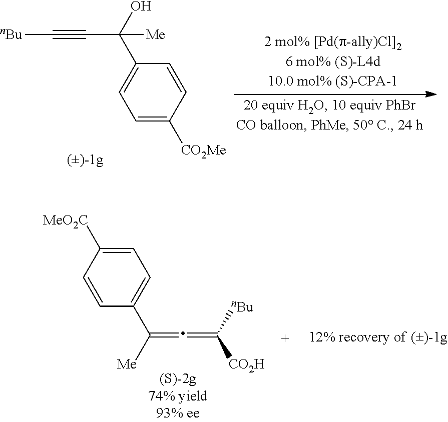

Operations were conducted by referring to Example 1. [Pd(π-allyl)Cl]₂ (0.0016 g, 0.004 mmol), chiral bisphosphine ligand (S)-L4d (0.0165 g, 0.0012 mmol), (S)-CPA-1 (0.0159 g, 0.02 mmol), (±)-1g (0.0519 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2g (0.0427 g, 74%): white solid; 94% ee (HPLC conditions: AS-H column, hexane/ⁱPrOH=98/2, 1.0 mL/min, λ=214 nm, t_{R}(major)=10.5 min, t_{R}(minor)=14.8 min); ¹H NMR (400 MHz, CDCl₃): δ=7.50-7.41 (m, 2 H, Ar—H), 7.26-7.19 (m, 2 H, Ar—H), 2.32 (d, J=7.4 Hz, 2 H, CH₂), 2.16 (s, 3 H, CH₃), 1.49-1.39 (m, 2 H, CH₂), 1.39-1.29 (m, 2 H, CH₂), 0.88 (t, J=7.2 Hz, 3 H, CH₃); ¹³C NMR (100 MHz, CDCl₃): δ=212.4, 172.4, 134.1, 131.7, 127.6, 121.5, 104.5, 102.2, 30.2, 28.2, 22.2, 16.2, 13.8.

### EXAMPLE 8

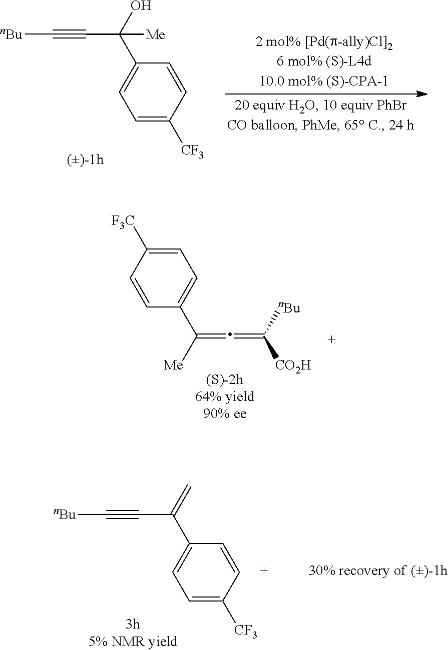

Operations were conducted by referring to Example 1. [Pd(π-allyl)Cl]₂ (0.0037 g, 0.01 mmol), chiral bisphosphine ligand (S)-L4d (0.0368 g, 0.03 mmol), (S)-CPA-1 (0.0403 g, 0.05 mmol), (±)-1h (0.1349 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 65° C. for 24 hours. Flash column chromatography (eluent: petroleum ether (60˜90° C.)/ethyl acetate=10/1, then 15/1) to afford a product: chiral allenoic acid (S)-2h (0.0911 g, 73%): white solid; 90% ee (HPLC conditions: AD-H column, hexane/ⁱPrOH=99/1, 1.0 mL/min, λ=214 nm, t_{R}(minor)=17.8 min, t_{R}(major)=27.0 min); [α]_{D}²⁶=+19.2 (c=1.00, CHCl₃); melting point: 101.4-102.4° C. (petroleum ether/DCM); ¹H NMR (400 MHz, CDCl₃): δ=7.59 (d, J=8.4 Hz, 2 H, Ar—H), 7.48 (d, J=8.4 Hz, 2 H, Ar—H), 2.34 (t, J=7.6 Hz, 2 H, CH₂), 2.21 (s, 3 H, CH₃), 1.51-1.41 (m, 2 H, CH₂), 1.40-1.30 (m, 2 H, CH₂), 0.88 (t, J=7.2 Hz, 3 H, CH₃); ¹³C NMR (100 MHz, CDCl₃): δ=212.9, 172.5, 139.0, 129.5 (q, J=32.4 Hz), 126.3, 125.5 (q, J=3.7 Hz), 124.1 (q, J=270.2 Hz), 104.5, 102.5, 30.2, 28.2, 22.2, 16.2, 13.8; ¹⁹F NMR (376 MHz, CDCl₃): δ=−63.1; IR (neat): ν=2957, 2939, 2867, 1943, 1689, 1418, 1327, 1267, 1125, 1075 cm⁻¹; MS (70 eV, EI) m/z (%): 299 (M⁺+1, 1.65), 298 (M⁺, 9.88), 211 (100); Anal. Calcd. for C₁₆H₁₇F₃O₂: C 64.42, H 5.74; found C 64.60, H 5.87.

### EXAMPLE 9

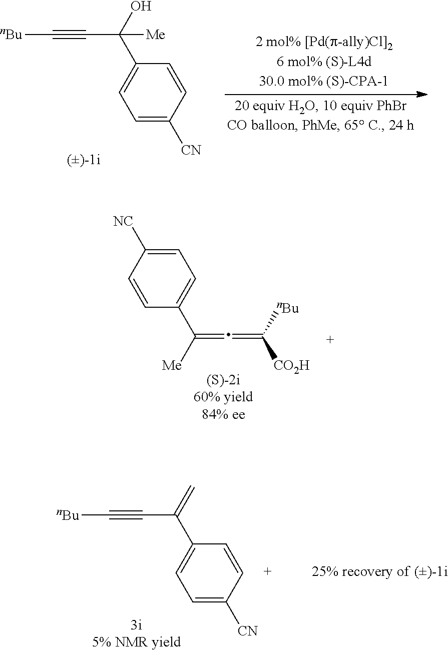

Operations were conducted by referring to Example 1. [Pd(π-allyl)Cl]₂ (0.0036 g, 0.01 mmol), chiral bisphosphine ligand (S)-L4d (0.0369 g, 0.03 mmol), (S)-CPA-1 (0.1202 g, 0.15 mmol), (±)-1i (0.1137 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 65° C. for 24 hours. Flash column chromatography (eluent: petroleum ether (60˜90° C.)/ether/dichloromethane=10/1/1, petroleum ether (60˜90° C.)/ethyl acetate=15/1) to afford a product: chiral allenoic acid (S)-21 (0.0772 g, 60%): white solid; 84% ee (HPLC conditions: AS-H column, hexane/ⁱPrOH=90/10, 1.0 mL/min, λ=214 nm, t_{R}(minor)=10.7 min, t_{R}(major)=12.8 min); [α]_{D}²⁵=+17.1 (c=1.00, CHCl₃); melting point: (petroleum ether/DCM); ¹H NMR (400 MHz, CDCl₃): δ=7.63 (d, J=8.4 Hz, 2 H, Ar—H), 7.47 (d, J=8.4 Hz, 2 H, Ar—H), 2.35 (t, J=7.6 Hz, 2 H, CH₂), 2.20 (s, 3 H, CH₃), 1.50-1.40 (m, 2 H, CH₂), 1.40-1.29 (m, 2 H, CH₂), 0.88 (t, J=7.2 Hz, 3 H, CH₃); ¹³C NMR (100 MHz, CDCl₃): δ=213.1, 172.2, 140.1, 132.3, 126.5, 118.7, 110.9, 104.4, 102.8, 30.1, 28.2, 22.2, 16.0, 13.7; IR (neat): ν=2962, 2930, 2862, 2227, 1939, 1693, 1419, 1285, 1059 cm⁻¹; MS (70 eV, EI) m/z (%): 256 (M⁺+1, 1.41), 255 (M⁺, 4.50), 168 (100); Anal. Calcd. for C₁₆H₁₇NO₂: C 75.27, H 6.71; found C 75.16, H 6.65.

### EXAMPLE 10

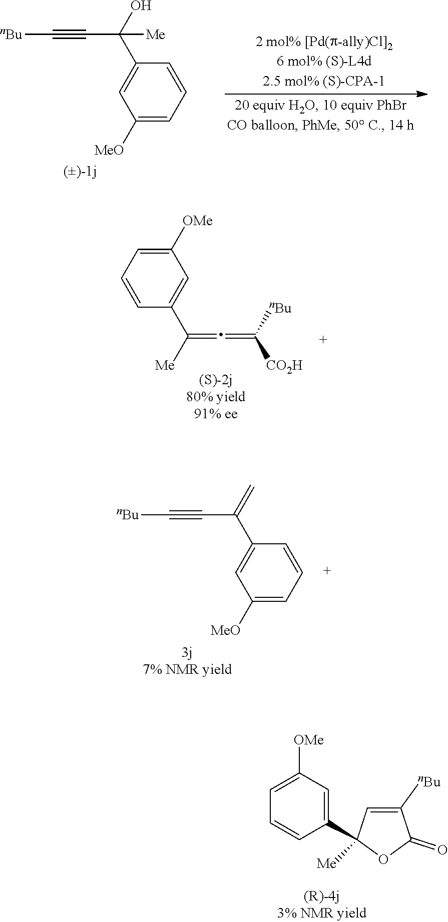

Operations were conducted by referring to Example 1. [Pd(π-allyl)Cl]₂ (0.0016 g, 0.004 mmol), chiral bisphosphine ligand (S)-L4d (0.0149 g, 0.0012 mmol), (S)-CPA-1 (0.004 g, 0.005 mmol), (±)-1j (0.0465 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 14 hours. Flash column chromatography (eluent: petroleum ether (60˜90° C.)/ethyl acetate=15/1, then 10/1) to afford a product: chiral allenoic acid (S)-2j (0.0416 g, 80%): white solid; 91% ee (HPLC conditions: AS-H column, hexane/ⁱPrOH=98/2, 1.0 mL/min, λ=214 nm, t_{R}(major)=11.9 min, t_{R}(minor)=16.1 min); ¹H NMR (400 MHz, CDCl₃): δ=7.26 (t, J=8.0 Hz, 1 H, Ar—H), 6.98 (d, J=8.0 Hz, 1 H, Ar—H), 6.92 (t, J=2.0 Hz, 1 H, Ar—H), 6.81 (dd, Ji =8.4 Hz, J₂=2.4 Hz, 1 H, Ar—H), 3.81 (s, 3 H, OCH₃), 2.32 (t, J=7.6 Hz, 2 H, CH₂), 2.18 (s, 3 H, CH₃), 1.52-1.41 (m, 2 H, CH₂), 1.41-1.30 (m, 2 H, CH₂), 0.88 (t, J=7.4 Hz, 3 H, CH₃); ¹³C NMR (100 MHz, CDCl₃): δ=212.5, 172.4, 159.8, 136.6, 129.5, 118.6, 112.8, 112.0, 105.1, 101.8, 55.2, 30.2, 28.3, 22.3, 16.4, 13.8.

### EXAMPLE 11

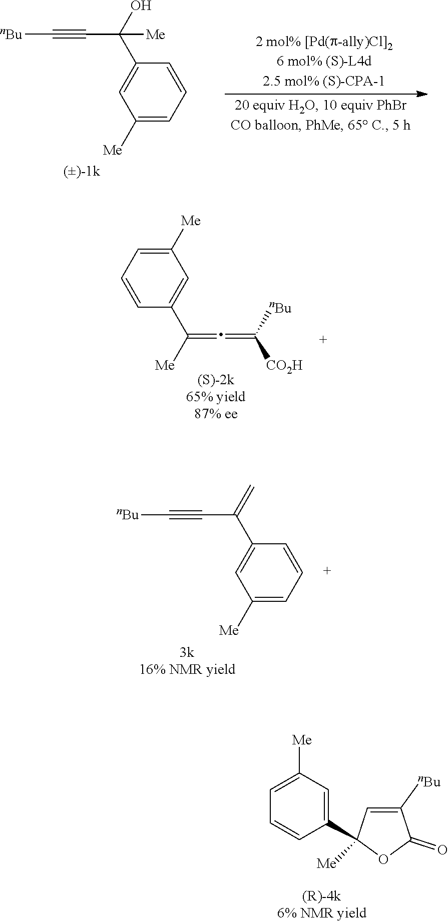

Operations were conducted by referring to Example 1. [Pd(π-allyl)Cl]₂ (0.0016 g, 0.004 mmol), chiral bisphosphine ligand (S)-L4d (0.0148 g, 0.0012 mmol), (S)-CPA-1 (0.0041 g, 0.005 mmol), (±)-1k (0.0432 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 65° C. for 5 hours. Flash column chromatography (eluent: petroleum ether (60˜90° C.)/ethyl acetate=20/1, then 15/1) to afford a product: chiral allenoic acid (S)-2k (0.0319 g, 65%): white solid; 87% ee (HPLC conditions: AS-H column, hexane/ⁱPrOH=98/2, 1.0 mL/min, λ=214 nm, t_{R}(major)=7.3 min, t_{R}(minor)=9.6 min); ¹H NMR (400 MHz, CDCl₃): δ=7.29-7.12 (m, 3 H, Ar—H), 7.07 (d, J=7.2 Hz, 1 H, Ar—H), 2.41-2.27 (m, 5 H, CH₂ and CH₃), 2.18 (s, 3 H, CH₃), 1.52-1.41 (m, 2 H, CH₂), 1.40-1.29 (m, 2 H, CH₂), 0.88 (t, J=7.4 Hz, 3 H, CH₃); ¹³C NMR (100 MHz, CDCl₃): δ=212.5, 172.7, 138.1, 134.9, 128.42, 128.38, 126.7, 123.2, 105.2, 101.6, 30.2, 28.3, 22.3, 21.5, 16.4, 13.8.

### EXAMPLE 12

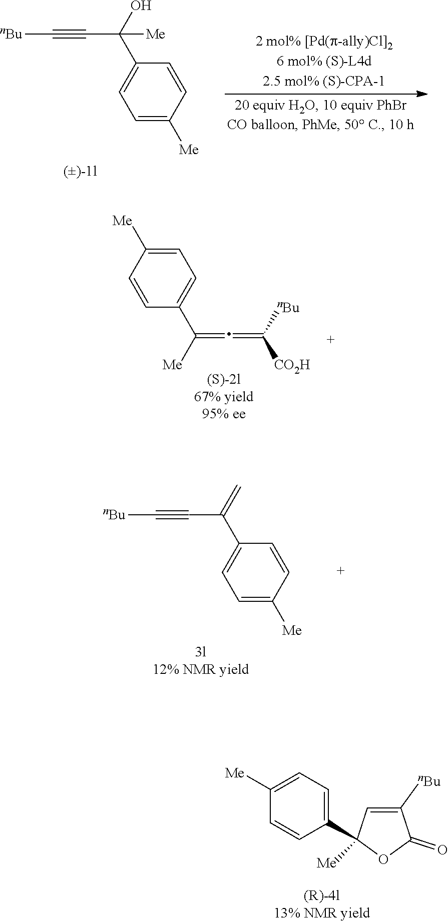

Operations were conducted by referring to Example 1. [Pd(π-allyl)Cl]₂ (0.0016 g, 0.004 mmol), chiral bisphosphine ligand (S)-L4d (0.0147 g, 0.0012 mmol), (S)-CPA-1 (0.0039 g, 0.005 mmol), (±)-1l (0.043 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 10 hours. Flash column chromatography (eluent: petroleum ether (60˜90° C.)/ethyl acetate=20/1, then 15/1) to afford a product: chiral allenoic acid (S)-2l (0.0325 g, 67%): white solid; 95% ee (HPLC conditions: AS-H column, hexane/ⁱPrOH=98/2, 1.0 mL/min, λ=214 nm, t_{R}(major)=9.4 min, t_{R}(minor)=10.9 min); ¹H NMR (400 MHz, CDCl₃): δ=7.27 (d, J=8.0 Hz, 2 H, Ar—H), 7.15 (d, J=8.0 Hz, 2 H, Ar—H), 2.38-2.26 (m, 5 H, CH₂ and CH₃), 2.17 (s, 3 H, CH₃), 1.50-1.40 (m, 2 H, CH₂), 1.39-1.29 (m, 2 H, CH₂), 0.87 (t, J=7.2 Hz, 3 H, CH₃); ¹³C NMR (100 MHz, CDCl₃): δ=212.5, 172.9, 137.4, 132.0, 129.2, 126.0, 105.1, 101.7, 30.2, 28.3, 22.3, 21.1, 16.3, 13.8.

### EXAMPLE 13

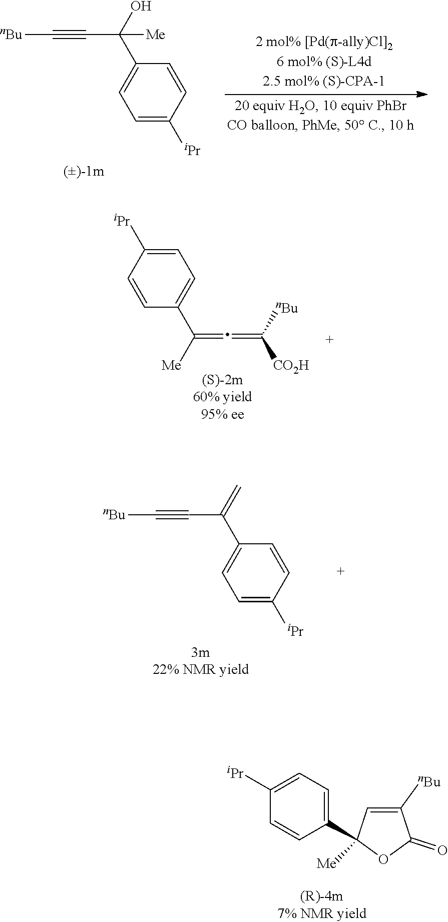

Operations were conducted by referring to Example 1. [Pd(π-allyl)Cl]₂ (0.0038 g, 0.01 mmol), chiral bisphosphine ligand (S)-L4d (0.0369 g, 0.03 mmol), (S)-CPA-1 (0.0101 g, 0.0125 mmol), (±)-1m (0.1223 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 10 hours. Flash column chromatography (eluent: petroleum ether (60˜90° C.)/ethyl acetate=15/1) to afford a product: chiral allenoic acid (S)-2m (0.0821 g, 60%): white solid; 95% ee (HPLC conditions: AD-H column, hexane/ⁱPrOH=99/1, 1.0 mL/min, λ=214 nm, t_{R}(major)=16.7 min, t_{R}(minor)=18.6 min); [α]_{D}²⁷=+20.7 (c=1.01, CHCl₃); melting point: 79.6-80.2° C. (petroleum ether/DCM); ¹H NMR (400 MHz, CDCl₃): δ=7.31 (d, J=8.4 Hz, 2 H, Ar—H), 7.20 (d, J=8.4 Hz, 2 H, Ar—H), 2.90 (heptet, J=6.8 Hz, 1 H, CH), 2.32 (t, J=7.6 Hz, 2 H, CH₂), 2.17 (s, 3 H, CH₃), 1.51-1.40 (m, 2 H, CH₂), 1.40-1.29 (m, 2 H, CH₂), 1.24 (d, J=6.8 Hz, 6 H, 2 x CH₃), 0.88 (t, J=7.2 Hz, 3 H, CH₃); ¹³C NMR (100 MHz, CDCl₃): δ=212.6, 172.9, 148.4, 132.4, 126.6, 126.0, 105.0, 101.7, 33.8, 30.2, 28.3, 23.90, 23.87, 22.3, 16.3, 13.8; IR (neat): ν=2958, 2927, 1941, 1679, 1419, 1278, 1067 cm⁻¹; MS (70 eV, EI) m/z (%): 272 (M⁺, 3.98), 143 (100); Anal. Calcd. for C₁₈H₂₄O₂: C 79.37, H 8.88; found C 79.32, H 8.82.

### EXAMPLE 14

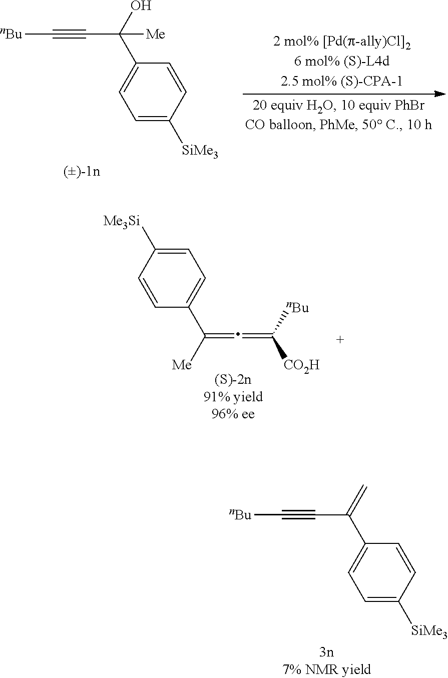

Operations were conducted by referring to Example 1. [Pd(π-allyl)Cl]₂ (0.0036 g, 0.01 mmol), chiral bisphosphine ligand (S)-L4d (0.0367 g, 0.03 mmol), (S)-CPA-1 (0.0102 g, 0.0125 mmol), (±)-1n (0.1375 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 10 hours. Flash column chromatography (eluent: petroleum ether (60˜90° C.)/ethyl acetate=15/1) to afford a product: chiral allenoic acid (S)-2n (0.1372 g, 91%): white solid; 96% ee (HPLC conditions: AD-H column, hexane/ⁱPrOH=99/1, 1.0 mL/min, λ=214 nm, t_{R}(major)=10.6 min, t_{R}(minor)=12.9 min); [α]_{D}²⁹=+20.3 (c=1.00, CHCl₃); melting point: 80.8-81.3° C. (petroleum ether/DCM); ¹H NMR (400 MHz, CDCl₃): δ=7.50 (d, J=8.0 Hz, 2 H, Ar—H), 7.37 (d, J=8.4 Hz, 2 H, Ar—H), 2.32 (t, J=7.4 Hz, 2 H, CH₂), 2.18 (s, 3 H, CH₃), 1.53-1.40 (m, 2 H, CH₂), 1.40-1.29 (m, 2 H, CH₂), 0.88 (t, J=7.2 Hz, 3 H, CH₃), 0.26 (s, 9 H, 3 x CH₃); ¹³C NMR (100 MHz, CDCl₃): δ=213.7, 173.9, 140.9, 136.5, 134.6, 126.3, 106.2, 102.9, 31.2, 29.3, 23.3, 17.2, 14.8, −0.2; IR (neat): ν=2956, 2928, 1942, 1682, 1416, 1249, 1058 cm⁻¹; MS (70 eV, EI) m/z (%): 303 (M⁺+1, 1.80), 302 (M⁺, 7.35), 73 (100); Anal. Calcd. for C₁₈H₂₆O₂Si: C 71.47, H 8.66; found C 71.45, H 8.55.

### EXAMPLE 15

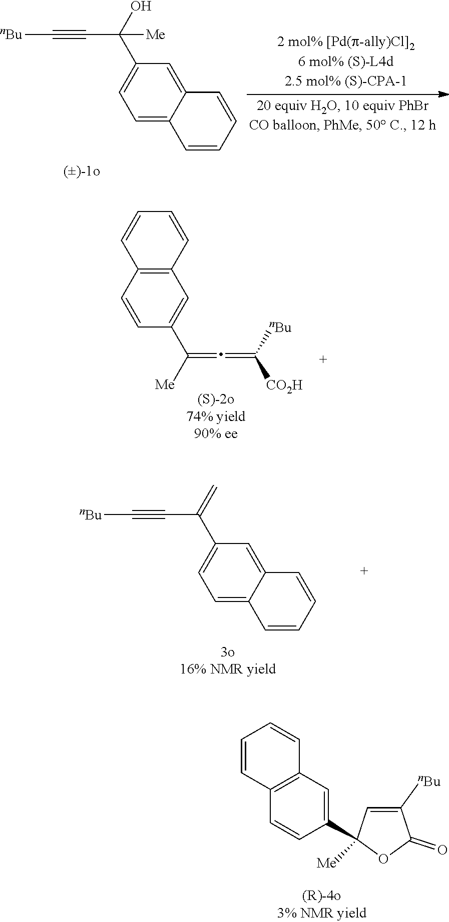

Operations were conducted by referring to Example 1. [Pd(π-allyl)Cl]₂ (0.0015 g, 0.004 mmol), chiral bisphosphine ligand (S)-L4d (0.0149 g, 0.0012 mmol), (S)-CPA-1 (0.0041 g, 0.005 mmol), (±)-1o (0.0503 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 12 hours. Flash column chromatography (eluent: petroleum ether (60˜90° C.)/ethyl acetate=15/1, then 10/1) to afford a product: chiral allenoic acid (S)-2o (0.0414 g, 74%): white solid; 90% ee (HPLC conditions: AS-H column, hexane/ⁱPrOH=98/2, 1.0 mL/min, λ=214 nm, t_{R}(major)=11.3 min, t_{R}(minor)=15.0 min); ¹H NMR (400 MHz, CDCl₃): δ=7.87-7.71 (m, 4 H, Ar—H), 7.56-7.40 (m, 3 H, Ar—H), 2.37 (t, J=7.4 Hz, 2 H, CH₂), 2.31 (s, 3 H, CH₃), 1.54-1.43 (m, 2 H, CH₂), 1.42-1.31 (m, 2 H, CH₂), 0.88 (t, J=7.2 Hz, 3 H, CH₃); ¹³C NMR (100 MHz, CDCl₃): δ=213.1, 172.4, 133.5, 132.8, 132.4, 128.09, 128.06, 127.6, 126.3, 126.1, 124.8, 124.2, 105.5, 102.1, 30.2, 28.4, 22.3, 16.3, 13.8.

### EXAMPLE 16

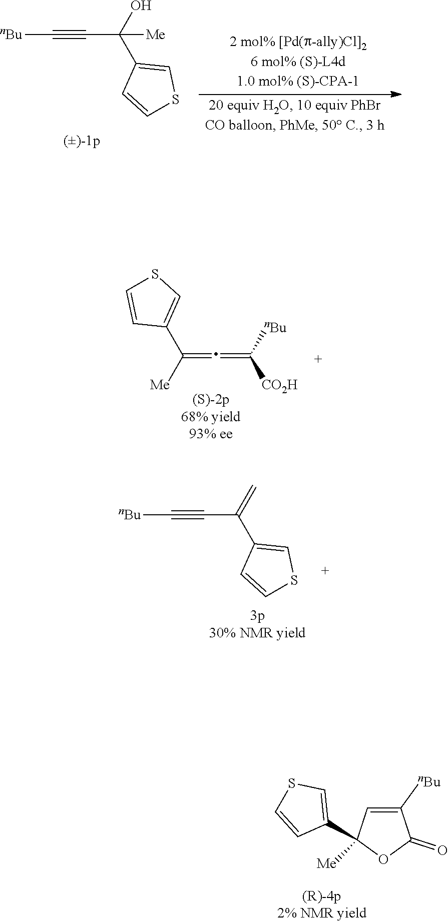

Operations were conducted by referring to Example 1. [Pd(π-allyl)Cl]₂ (0.0015 g, 0.004 mmol), chiral bisphosphine ligand (S)-L4d (0.0148 g, 0.0012 mmol), (S)-CPA-1 (0.0015 g, 0.002 mmol), (±)-1p (0.0503 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 3 hours. Flash column chromatography (eluent: petroleum ether (60˜90° C.)/ethyl acetate=15/1, then 10/1) to afford a product: chiral allenoic acid (S)-2p (0.0321 g, 68%): white solid; 93% ee (HPLC conditions: AS-H column, hexane/ⁱPrOH=98/2, 1.0 mL/min, λ=214 nm, t_{R}(major)=11.5 min, t_{R}(minor)=15.9 min); ¹H NMR (400 MHz, CDCl₃): δ=7.28 (dd, J₁=5.2 Hz, J₂=2.8 Hz, 1 H, one proton from thienyl), 7.15 (d, J₁=2.8 Hz, J₂=1.2 Hz, 1 H, one proton from thienyl), 7.04 (d, J₁=5.0 Hz, J₂=1.0 Hz, 1 H, one proton from thienyl), 2.31 (t, J=7.4 Hz, 2 H, CH₂), 2.17 (s, 3H, CH₃), 1.50-1.40 (m, 2H, CH₂), 1.40-1.30 (m, 2 H, CH₂), 0.88 (t, J=7.2 Hz, 3 H, CH₃); ¹³C NMR (100 MHz, CDCl₃): δ=212.8, 172.2, 136.5, 126.3, 125.9, 120.6, 101.4, 101.3, 30.3, 28.4, 22.2, 16.7, 13.8.

### EXAMPLE 17

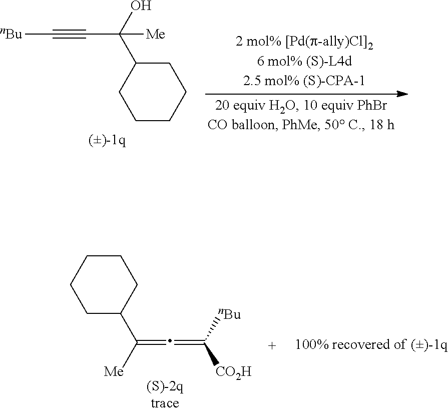

Operations were conducted by referring to Example 1. [Pd(π-allyl)Cl]₂ (0.0016 g, 0.004 mmol), chiral bisphosphine ligand (S)-L4d (0.0149 g, 0.0012 mmol), (S)-CPA-1 (0.004 g, 0.002 mmol), (±)-1q (0.042 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. No target chiral allenoic acid product (S)-2q was formed.

### EXAMPLE 18

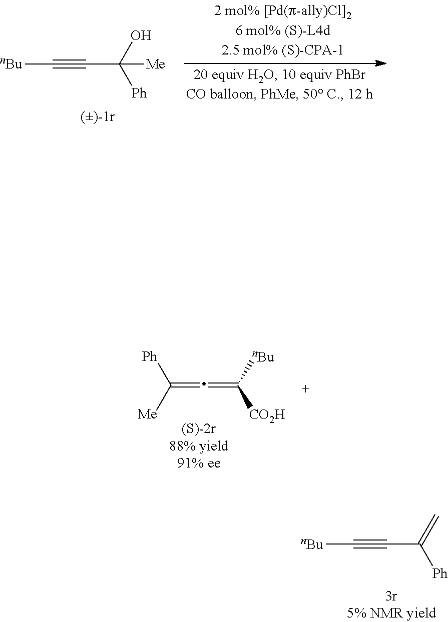

Operations were conducted by referring to Example 1. [Pd(π-allyl)Cl]₂ (0.0037 g, 0.01 mmol), chiral bisphosphine ligand (S)-L4d (0.0369 g, 0.03 mmol), (S)-CPA-1 (0.0101 g, 0.0125 mmol), (±)-1r (0.0943 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 15/1) to afford a product: chiral allenoic acid (S)-2r (0.0948 g, 88%): white solid; 91% ee (HPLC conditions: AS-H column, hexane/ⁱPrOH=98/2, 1.0 mL/min, λ=214 nm, t_{R}(major)=9.4 min, t_{R}(minor)=12.7 min); [α]_{D}²⁶=+49.4 (c=1.01, CHCl₃); melting point: 88.5-89.6° C. (petroleum ether/DCM); ¹H NMR (400 MHz, CDCl₃): δ=7.38 (d, J=7.6 Hz, 2 H, Ar—H), 7.33 (t, J=7.4 Hz, 2 H, Ar—H), 7.24 (t, J=7.2 Hz, 1 H, Ar—H), 2.30 (t, J=7.6 Hz, 2 H, CH₂), 2.19 (s, 3 H, CH₃), 1.51 (sextet, J=7.4 Hz, 2 H, CH₂), 0.92 (t, J=7.2 Hz, 3 H, CH₃); ¹³C NMR (100 MHz, CDCl₃): δ=212.7, 173.0, 135.0, 128.5, 127.5, 126.1, 105.2, 101.6, 30.6, 21.4, 16.3, 13.7; IR (neat): ν=2961, 2929, 1942, 1682, 1415, 1263, 1066 cm⁻¹; MS (70 eV, EI) m/z (%): 217 (M⁺+1, 3.86), 216 (M⁺, 24.20), 143 (100); Anal. Calcd. for Ci4H1602: C 77.75, H 7.46; found C 77.89, H 7.63.

### EXAMPLE 19

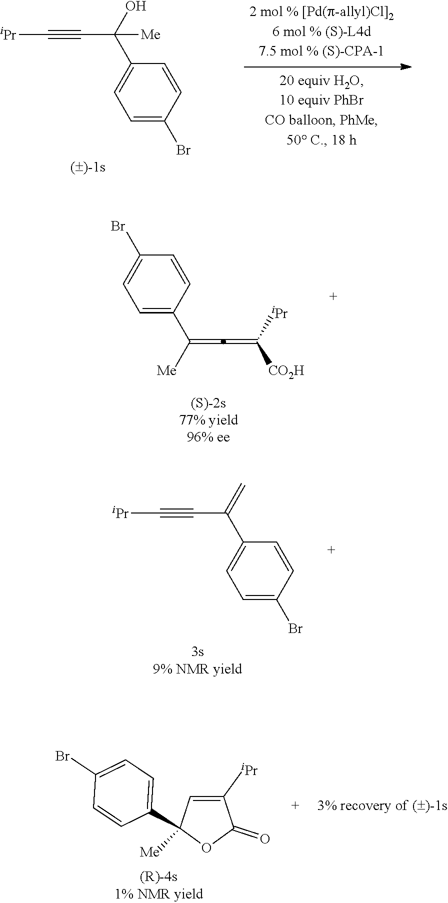

Operations were conducted by referring to Example 1. [Pd(π-allyl)Cl]₂ (0.0016 g, 0.004 mmol), chiral bisphosphine ligand (S)-L4d (0.0148 g, 0.0012 mmol), (S)-CPA-1 (0.0118 g, 0.015 mmol), (±)-1s (0.0531 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2s (0.0449 g, 77%): white solid; 96% ee (HPLC conditions: AS-H column, hexane/ⁱPrOH=98/2, 0.5 mL/min, λ=214 nm, t_{R}(major)=23.1 min, t_{R}(minor)=26.0 min); ¹H NMR (400 MHz, CDCl₃): δ=7.46 (d, J=8.4 Hz, 2 H, Ar—H), 7.25 (d, J=8.4 Hz, 2 H, Ar—H), 2.79 (heptet, J=6.8 Hz, 1 H, CH), 2.17 (s, 3 H, CH₃), 1.09 (d, J=6.8 Hz, 6 H, 2 x CH₃); ¹³C NMR (100 MHz, CDCl₃): δ=211.2, 171.9, 134.0, 131.7, 127.5, 121.5, 109.0, 105.9, 28.2, 22.1, 22.0, 16.3.

### EXAMPLE 20

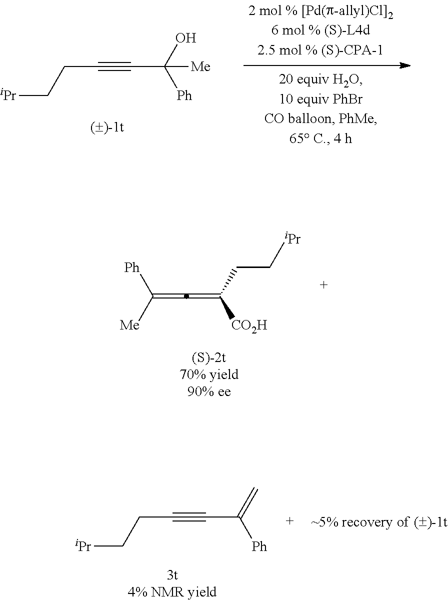

Operations were conducted by referring to Example 1. [Pd(π-allyl)Cl]₂ (0.0015 g, 0.004 mmol), chiral bisphosphine ligand (S)-L4d (0.0149 g, 0.0012 mmol), (S)-CPA-1 (0.004 g, 0.015 mmol), (±)-1t (0.0346 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 65° C. for 4 hours. Flash column chromatography (eluent: petroleum ether (60˜90° C.)/ethyl acetate=15/1, then 10/1) to afford a product: chiral allenoic acid (S)-2t (0.0346 g, 70%): white solid; 90% ee (HPLC conditions: AS-H column, hexane/ⁱPrOH=98/2, 1.0 mL/min, λ=214 nm, t_{R}(major)=7.5 min, t_{R}(minor)=9.9 min); ¹H NMR (400 MHz, CDCl₃): δ=7.44-7.28 (m, 4 H, Ar—H), 7.28-7.22 (m, 1 H, Ar—H), 2.33 (t, J=8.0 Hz, 2 H, CH₃), 2.19 (s, 3 H, CH₃), 1.65-1.50 (m, 1 H, CH), 1.42-1.30 (m, 2 H, CH₂), 0.87 (t, J=6.0 Hz, 6 H, 2 x CH₃); ¹³C NMR (100 MHz, CDCl₃): δ=212.4, 172.6, 135.0, 128.5, 127.6, 126.1, 105.3, 102.0, 37.1, 27.7, 26.6, 22.44, 22.40, 16.3.

### EXAMPLE 21

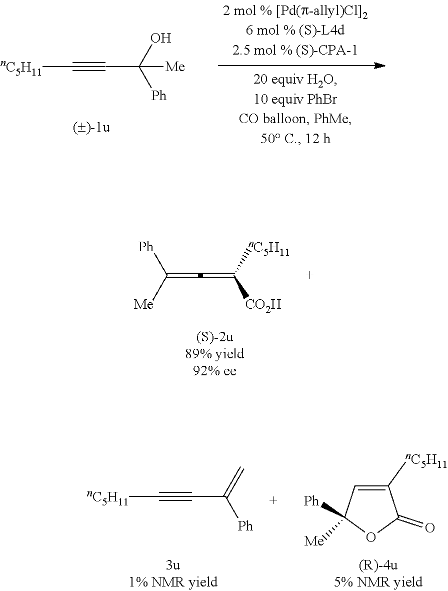

Operations were conducted by referring to Example 1. [Pd(π-allyl)Cl]₂ (0.0015 g, 0.004 mmol), chiral bisphosphine ligand (S)-L4d (0.0147 g, 0.0012 mmol), (S)-CPA-1 (0.0041 g, 0.015 mmol), (±)-1u (0.0431 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 12 hours. Flash column chromatography (eluent: petroleum ether (60˜90° C.)/ethyl acetate=15/1, then 10/1) to afford a product: chiral allenoic acid (S)-2u (0.0434 g, 89%): white solid; 92% ee (HPLC conditions: AS-H column, hexane/ⁱPrOH=98/2, 1.0 mL/min, λ=214 nm, t_{R}(major)=7.8 min, t_{R}(minor)=12.9 min); ¹H NMR (400 MHz, CDCl₃): δ=7.44-7.30 (m, 4 H, Ar—H), 7.28-7.22 (m, 1 H, Ar—H), 2.32 (t, J=7.6 Hz, 2 H, CH₂), 2.19 (s, 3 H, CH₃), 1.54-1.41 (m, 2 H, CH₂), 1.33-1.23 (m, 4 H, 2 x CH₂), 0.84 (t, J=7.0 Hz, 3 H, CH₃); ¹³C NMR (100 MHz, CDCl₃): δ=212.6, 172.7, 135.0, 128.5, 127.6, 126.1, 105.2, 101.8, 31.3, 28.5, 27.7, 22.4, 16.3, 14.0.

### EXAMPLE 22

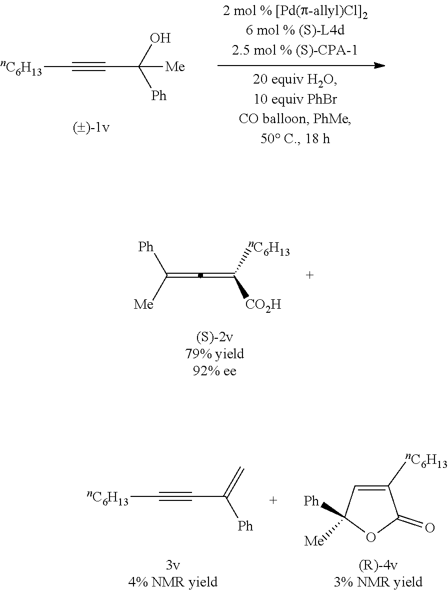

Operations were conducted by referring to Example 1. [Pd(π-allyl)Cl]₂ (0.0015 g, 0.004 mmol), chiral bisphosphine ligand (S)-L4d (0.0148 g, 0.0012 mmol), (S)-CPA-1 (0.0041 g, 0.015 mmol), (±)-1v (0.0485 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2v (0.0405 g, 79%): white solid; 92% ee (HPLC conditions: AS-H column, hexane/ⁱPrOH=98/2, 1.0 mL/min, λ=214 nm, t_{R}(major)=7.3 min, t_{R}(minor)=12.2 min); ¹H NMR (400 MHz, CDCl₃): δ=7.41-7.29 (m, 4 H, Ar—H), 7.28-7.22 (m, 1 H, Ar—H), 2.32 (t, J=7.4 Hz, 2 H, CH₂), 2.19 (s, 3H, CH₃), 1.53-1.41 (m, 2 H, CH₂), 1.36-1.15 (m, 6 H, 3 x CH₂), 0.84 (t, J=6.8 Hz, 3 H, CH₃); ¹³C NMR (100 MHz, CDCl₃): δ=212.6, 172.7, 135.0, 128.5, 127.6, 126.1, 105.2, 101.8, 31.6, 28.8, 28.6, 28.0, 22.6, 16.3, 14.0.

### EXAMPLE 23

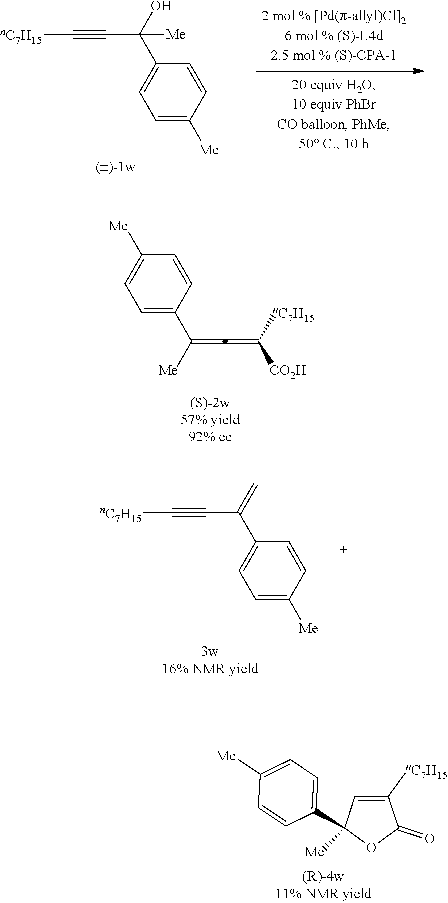

Operations were conducted by referring to Example 1. [Pd(π-allyl)Cl]₂ (0.0038 g, 0.01 mmol), chiral bisphosphine ligand (S)-L4d (0.0367 g, 0.03 mmol), (S)-CPA-1 (0.0101 g, 0.0125 mmol), (±)-1w (0.1292 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 10 hours. Flash column chromatography (eluent: petroleum ether (60˜90° C.)/ethyl acetate=15/1) to afford a product: chiral allenoic acid (S)-2w (0.0812 g, 57%): white solid; 92% ee (HPLC conditions: AS-H column, hexane/ⁱPrOH=98/2, 1.0 mL/min, λ=214 nm, t_{R}(major)=7.2 min, t_{R}(minor)=9.6 min); [α]_{D}³¹=+3.0 (c=1.00, CHCl₃); melting point: 81.4-82.4° C. (petroleum ether/DCM); ¹H NMR (400 MHz, CDCl₃): δ=7.26 (d, J=8.4 Hz, 2 H, Ar—H), 7.14 (d, J=8.0 Hz, 2 H, Ar—H), 2.38-2.26 (m, 5 H, CH₂ and CH₃), 2.17 (s, 3 H, CH₃), 1.52-1.41 (m, 2 H, CH₂), 1.35-1.16 (m, 8 H, 4 x CH₂), 0.85 (t, J=6.8 Hz, 3 H, CH₃); ¹³C NMR (100 MHz, CDCl₃): δ=212.5, 172.9, 137.4, 132.0, 129.2, 126.0, 105.1, 101.6, 31.8, 29.14, 29.07, 28.6, 28.1, 22.6, 21.1, 16.3, 14.0; IR (neat): ν=2955, 2926, 2856, 1941, 1681, 1417, 1278, 1063 cm⁻¹; MS (70 eV, EI) m/z (%): 287 (M⁺+1, 2.80), 286 (M⁺, 6.61), 157 (100); Anal. Calcd. for C₁₉H₂₆O₂: C 79.68, H 9.15; found C 79.78, H 9.18.

### EXAMPLE 24

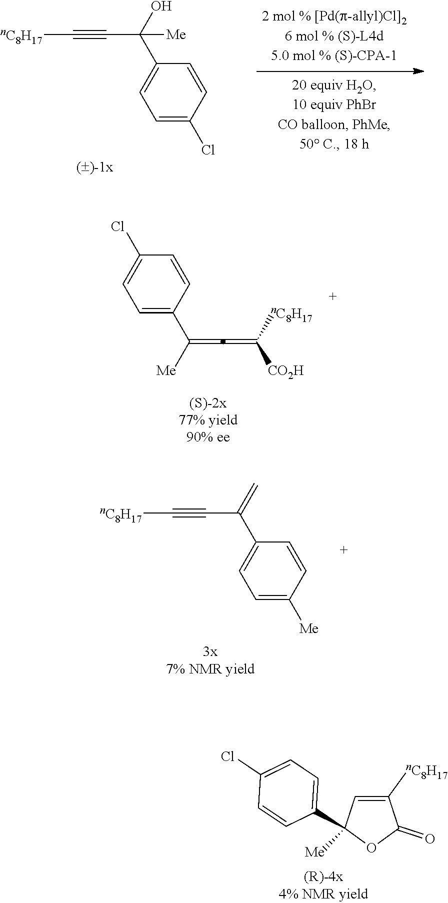

Operations were conducted by referring to Example 1. [Pd(π-allyl)Cl]₂ (0.0015 g, 0.004 mmol), chiral bisphosphine ligand (S)-L4d (0.0147 g, 0.0012 mmol), (S)-CPA-1 (0.0081 g, 0.01 mmol), (±)-1x (0.0585 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2x (0.0495 g, 77%): white solid; 90% ee (HPLC conditions: AS-H column, hexane/ⁱPrOH=98/2, 1.0 mL/min, λ=214 nm, t_{R}(major)=6.4 min, t_{R}(minor)=9.7 min); ¹H NMR (400 MHz, CDCl₃): δ=7.30 (s, 4 H, Ar—H), 2.31 (t, J=7.4 Hz, 2 H, CH₂), 2.17 (s, 3 H, CH₃), 1.51-1.39 (m, 2 H, CH₂), 1.34-1.17 (m, 10 H, 5 x CH₂), 0.86 (t, J=6.8 Hz, 3 H, CH₃); ¹³C NMR (100 MHz, CDCl₃): δ=212.5, 172.5, 133.6, 133.4, 128.7, 127.3, 104.4, 102.2, 31.8, 29.3, 29.2, 29.1, 28.5, 28.0, 22.6, 16.3, 14.0.

### EXAMPLE 25

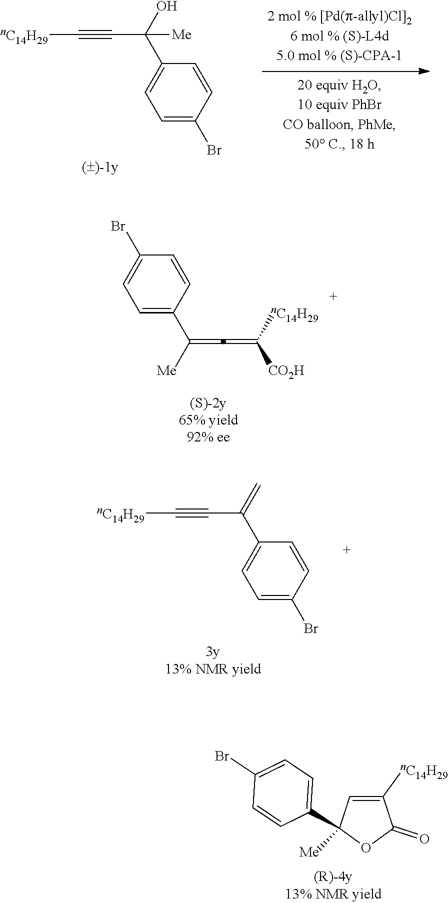

Operations were conducted by referring to Example 1. [Pd(π-allyl)Cl]₂ (0.0036 g, 0.01 mmol), chiral bisphosphine ligand (S)-L4d (0.037 g, 0.03 mmol), (S)-CPA-1 (0.02 g, 0.025 mmol), (±)-1y (0.211 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1) to afford a product: chiral allenoic acid (S)-2y (0.1459 g, 65%): white solid; 92% ee (HPLC conditions: AS-H column, hexane/ⁱPrOH=98/2, 0.5 mL/min, λ=214 nm, t_{R}(major)=10.2 min, t_{R}(minor)=14.5 min); [α]_{D}²⁵=−3.4 (c=1.04, CHCl₃); melting point: 81.1-81.6° C. (petroleum ether/DCM); ¹H NMR (400 MHz, CDCl₃): δ=7.45 (d, J=8.4 Hz, 2 H, Ar—H), 7.23 (d, J=8.4 Hz, 2 H, Ar—H), 2.31 (t, J=7.4 Hz, 2 H, CH₂), 2.16 (s, 3 H, CH₃), 1.51-1.39 (m, 2 H, CH₂), 1.36-1.12 (m, 22 H, 11 x CH₂), 0.88 (t, J=6.6 Hz, 3 H, CH₃); ¹³C NMR (100 MHz, CDCl₃): δ=212.5, 172.7, 134.1, 131.6, 127.6, 121.5, 104.5, 102.2, 31.9, 29.68, 29.66, 29.64, 29.59, 29.4, 29.3, 29.2, 28.5, 28.0, 22.7, 16.2, 14.1; IR (neat): ν=2921, 2854, 1940, 1685, 1475, 1417, 1271, 1079, 1017 cm⁻¹; MS (70 eV, EI) m/z (%): 450 (M⁺ (⁸¹Br), 4.83), 448 (M⁺ (⁷⁹Br), 4.76), 143 (100); Anal. Calcd. for C₂₅H₃₇BrO₂: C 66.81, H 8.30; found C 66.84, H 8.21.

### EXAMPLE 26

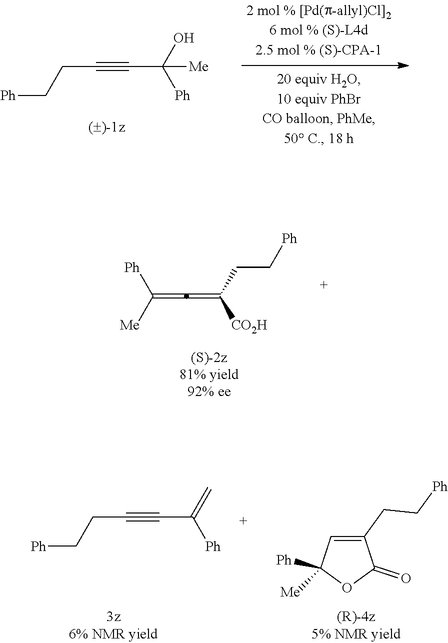

Operations were conducted by referring to Example 1. [Pd(π-allyl)Cl]₂ (0.0015 g, 0.004 mmol), chiral bisphosphine ligand (S)-L4d (0.0149 g, 0.0012 mmol), (S)-CPA-1 (0.004 g, 0.005 mmol), (±)-1z (0.0503 g, 0.2 mmol), bromobenzene (211 μL, d=1.49 g/mL, 0.3144 g, 2 mmol), water (72 μL, d=1.0 g/mL, 0.072 g, 4 mmol), Toluene (0.8 mL) were reacted at 50° C. for 18 hours. Flash column chromatography (eluent: petroleum ether (60˜90° C.)/ethyl acetate=15/1, then 10/1) to afford a product: chiral allenoic acid (S)-2z (0.0455 g, 81%): white solid; 92% ee (HPLC conditions: AS-H column, hexane/ⁱPrOH=98/2, 1.0 mL/min, λ=214 nm, t_{R}(major)=14.3 min, t_{R}(minor)=25.7 min); ¹H NMR (400 MHz, CDCl₃): δ=7.34-7.21 (m, 7 H, Ar—H), 7.19-7.13 (m, 3 H, Ar—H), 2.84 (t, J=7.6 Hz, 2 H, CH₃), 2.75-2.59 (m, 2 H, CH₂), 2.02 (s, 3 H, CH₃); ¹³C NMR (100 MHz, CDCl₃): δ=212.9, 172.6, 141.1, 134.7, 128.5, 128.3, 127.6, 126.1, 125.9, 105.5, 100.7, 34.0, 30.3, 16.1.

### EXAMPLE 27

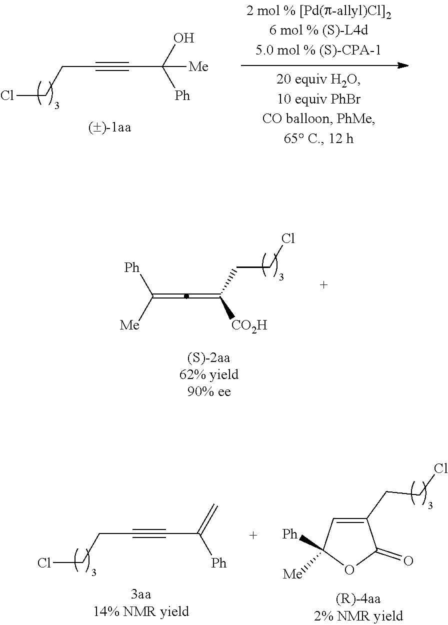

Operations were conducted by referring to Example 1. [Pd(π-allyl)Cl]₂ (0.0016 g, 0.004 mmol), chiral bisphosphine ligand (S)-L4d (0.0148 g, 0.0012 mmol), (S)-CPA-1 (0.0081 g, 0.01 mmol), (±)-1aa (0.0475 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 65° C. for 12 hours. Flash column chromatography (eluent: petroleum ether (60˜90° C.)/ethyl acetate=15/1, then 10/1) to afford a product: chiral allenoic acid (S)-2aa (0.0328 g, 62%): white solid; 90% ee (HPLC conditions: AS-H column, hexane/ⁱPrOH=98/2, 1.0 mL/min, λ=214 nm, t_{R}(major)=16.8 min, t_{R}(minor)=25.0 min); ¹H NMR (400 MHz, CDCl₃): δ=7.42-7.31 (m, 4 H, Ar—H), 7.29-7.24 (m, 1 H, Ar—H), 3.50 (t, J=6.6 Hz, 2 H, CH₂), 2.36 (t, J=7.6 Hz, 2 H, CH₂), 2.20 (s, 3 H, CH₃), 1.84-1.76 (m, 2 H, CH₂), 1.69-1.57 (m, 2 H, CH₂); ¹³C NMR (100 MHz, CDCl₃): δ=212.5, 172.6, 134.7, 128.6, 127.7, 126.1, 105.7, 101.1, 44.6, 32.0, 27.8, 25.3, 16.4.

### EXAMPLE 28

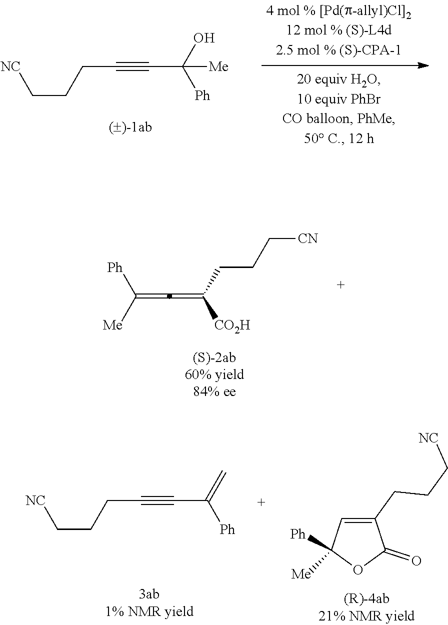

Operations were conducted by referring to Example 1. [Pd(π-allyl)Cl]₂ (0.0073 g, 0.02 mmol), chiral bisphosphine ligand (S)-L4d (0.0733 g, 0.06 mmol), (S)-CPA-1 (0.01 g, 0.0125 mmol), (±)-1ab (0.1069 g, 0.5 mmol), bromobenzene (527 μL, d=1.49 g/mL, 0.786 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate/dichloromethane=10/1/1, then petroleum ether (60˜90° C.)/ethyl acetate=3/1) to afford a product: chiral allenoic acid (S)-2ab (0.0328 g, 62%): white solid; 90% ee (HPLC conditions: AS-H column, hexane/ⁱPrOH=98/2, 1.0 mL/min, λ=214 nm, t_{R}(major)=16.8 min, t_{R}(minor)=25.0 min); ¹H NMR (400 MHz, CDCl₃): δ=7.42-7.31 (m, 4 H, Ar—H), 7.29-7.24 (m, 1 H, Ar—H), 3.50 (t, J=6.6 Hz, 2 H, CH₂), 2.36 (t, J=7.6 Hz, 2 H, CH₂), 2.20 (s, 3 H, CH₃), 1.84-1.76 (m, 2 H, CH₂), 1.69-1.57 (m, 2 H, CH₂); ¹³C NMR (100 MHz, CDCl₃): δ=212.5, 172.6, 134.7, 128.6, 127.7, 126.1, 105.7, 101.1, 44.6, 32.0, 27.8, 25.3, 16.4.

### EXAMPLE 29

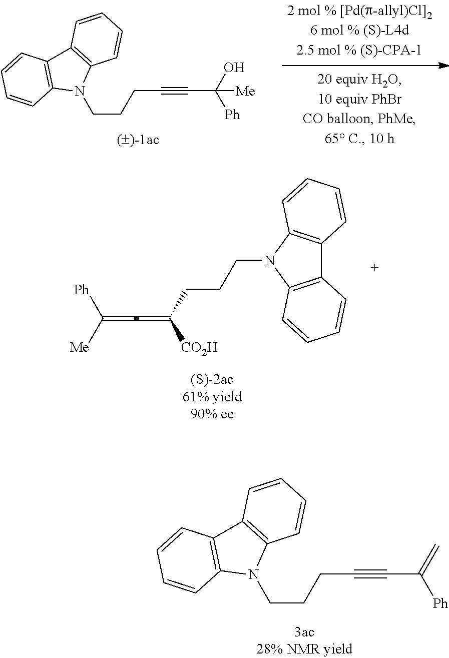

Operations were conducted by referring to Example 1. [Pd(π-allyl)Cl]₂ (0.0038 g, 0.01 mmol), chiral bisphosphine ligand (S)-L4d (0.0368 g, 0.03 mmol), (S)-CPA-1 (0.01 g, 0.0125 mmol), (±)-1ac (0.1775 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 65° C. for 10 hours. Flash column chromatography (eluent: petroleum ether (60˜90° C.)/ether/dichloromethane=10/1/1, then petroleum ether (60˜90° C.)/ethyl acetate=8/1) to afford a product: chiral allenoic acid (S)-2ac (0.1166 g, 61%): white solid; 90% ee (HPLC conditions: AS-H column, hexane/ⁱPrOH=90/10, 1.0 mL/min, λ=214 nm, t_{R}(major)=6.8 min, t_{R}(minor)=8.2 min); [α]_{D}²⁵=−31.4 (c=1.00, CHCl₃); melting point: 171.1-172.2° C. (petroleum ether/DCM); ¹H NMR (400 MHz, CDCl₃): δ=8.07 (d, J=7.6 Hz, 2 H, Ar—H), 7.43-7.23 (m, 9 H, Ar—H), 7.19 (t, J=7.4 Hz, 2 H, Ar—H), 4.30 (t, J=7.4 Hz, 2 H, CH₂), 2.46 (t, J=7.4 Hz, 2 H, CH₂), 2.20 (s, 3 H, CH₃), 2.12-1.98 (m, 2 H, CH₂); ¹³C NMR (100 MHz, CDCl₃): δ=212.2, 172.1, 140.3, 134.6, 128.7, 127.9, 126.1, 125.6, 122.8, 120.3, 118.8, 108.5, 106.3, 100.9, 42.6, 27.2, 26.3, 16.5; IR (neat): ν=3054, 2936, 1940, 1682, 1454, 1335, 1262, 1021 cm⁻¹; MS (70 eV, EI) m/z (%): 382 (M⁺+1, 7.06), 381 (M⁺, 20.11), 193 (100); Anal. Calcd. for C26H23NO2: C 81.86, H 6.08; found C 81.97, H 6.07.

### EXAMPLE 30

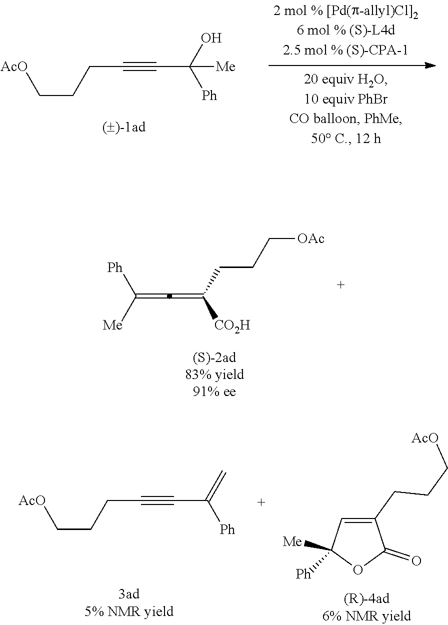

Operations were conducted by referring to Example 1. [Pd(π-allyl)Cl]₂ (0.0035 g, 0.01 mmol), chiral bisphosphine ligand (S)-L4d (0.0368 g, 0.03 mmol), (S)-CPA-1 (0.0101 g, 0.0125 mmol), (±)-1ad (0.1234 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate/dichloromethane=10/1/1, then petroleum ether (60˜90° C.)/ethyl acetate=8/1) to afford a product: chiral allenoic acid (S)-2ad (0.1134 g, 83%): oil substance; 91% ee (HPLC conditions: AS-H column, hexane/ⁱPrOH=90/10, 1.0 mL/min, λ=214 nm, t_{R}(major)=7.8 min, t_{R}(minor)=10.8 min); [α]_{D}²⁷=+7.5 (c=1.00, CHCl₃); ¹H NMR (400 MHz, CDCl₃): δ=7.45-7.29 (m, 4 H, Ar—H), 7.29-7.24 (m, 1 H, Ar—H), 4.09 (t, J=6.4 Hz, 2 H, CH₂), 2.42 (t, J=7.6 Hz, 2 H, CH₂), 2.21 (s, 3 H, CH₃), 2.02 (s, 3 H, CH₃), 1.88-1.76 (m, 2 H, CH₂); ¹³C NMR (100 MHz, CDCl₃): δ=212.3, 172.3, 171.2, 134.6, 128.6, 127.7, 126.0, 105.8, 100.8, 63.7, 27.0, 25.2, 20.8, 16.3; IR (neat): ν=2956, 2929, 1942, 1737, 1717, 1681, 1367, 1238, 1041 cm⁻¹; MS (70 eV, ESI) m/z: 297 (M+Na⁺), 275 (M+H⁺); HRMS calcd for C₁₆H₁₉O₄ [M+H⁺]: 275.1278, found: 275.1271.

### EXAMPLE 31

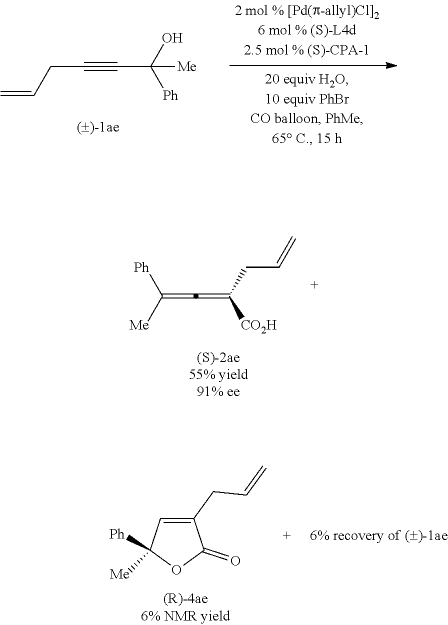

Operations were conducted by referring to Example 1. [Pd(π-allyl)Cl]₂ (0.0015 g, 0.004 mmol), chiral bisphosphine ligand (S)-L4d (0.0149 g, 0.0012 mmol), (S)-CPA-1 (0.0041 g, 0.005 mmol), (±)-1ae (0.0347 g, 0.2 mmol), bromobenzene (211 μL, d=1.49 g/mL, 0.3144 g, 2 mmol), water (72 μL, d=1.0 g/mL, 0.072 g, 4 mmol), toluene (0.8 mL) were reacted at 65° C. for 15 hours. Flash column chromatography (eluent: petroleum ether (60˜90° C.)/ethyl acetate=15/1, then 10/1) to afford a product: chiral allenoic acid (S)-2ae (0.0237 g, 55%): white solid; 90% ee (HPLC conditions: AS-H column, hexane/ⁱPrOH=98/2, 1.0 mL/min, λ=214 nm, t_{R}(major)=16.8 min, t_{R}(minor)=25.0 min); ¹H NMR (400 MHz, CDCl₃): δ=7.42-7.31 (m, 4 H, Ar—H), 7.29-7.24 (m, 1 H, Ar—H), 3.50 (t, J=6.6 Hz, 2 H, CH₂), 2.36 (t, J=7.6 Hz, 2 H, CH₂), 2.20 (s, 3 H, CH₃), 1.84-1.76 (m, 2 H, CH₂), 1.69-1.57 (m, 2 H, CH₂); ¹³C NMR (100 MHz, CDCl₃): δ=212.5, 172.6, 134.7, 128.6, 127.7, 126.1, 105.7, 101.1, 44.6, 32.0, 27.8, 25.3, 16.4.

### EXAMPLE 32

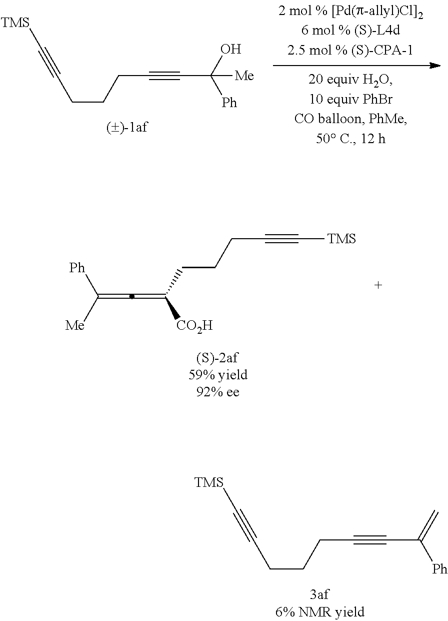

Operations were conducted by referring to Example 1. [Pd(π-allyl)Cl]₂ (0.0037 g, 0.01 mmol), chiral bisphosphine ligand (S)-L4d (0.0368 g, 0.03 mmol), (S)-CPA-1 (0.0101 g, 0.0125 mmol), (±)-1af (0.142 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 15/1) to afford a product: chiral allenoic acid (S)-2af (0.0904 g, 59%): white solid; 92% ee (HPLC conditions: AS-H column, hexane/ⁱPrOH=98/2, 0.5 mL/min, λ=214 nm, t_{R}(major)=13.1 min, t_{R}(minor)=16.7 min); [α]_{D}²⁵=−5.4 (c=1.00, CHCl₃); melting point: (petroleum ether/DCM); ¹H NMR (400 MHz, CDCl₃): δ=7.44-7.34 (m, 4 H, Ar—H), 7.32-7.27 (m, 1 H, Ar—H), 2.47 (td, J₁=7.5 Hz, J₂=2.0 Hz, 2 H, CH₂), 2.29 (t, J=7.2 Hz, 2 H, CH₂), 2.24 (s, 3 H, CH₃), 1.80-1.70 (m, 2 H, CH₂), 0.15 (s, 9 H, CH₃); ¹³C NMR (100 MHz, CDCl₃): δ=212.6, 172.6, 134.8, 128.6, 127.7, 126.1, 106.6, 105.6, 100.9, 84.9, 27.8, 26.9, 19.3, 16.4, 0.1; IR (neat): ν=2958, 2173, 1941, 1682, 1417, 1281, 1250, 1026 cm⁻¹; MS (70 eV, ESI) m/z: 313 (M+H⁺); Anal. Calcd. for C₁₉H₂₄O₂Si: C 73.03, H 7.74; found C 73.19, H 7.75.

### EXAMPLE 33

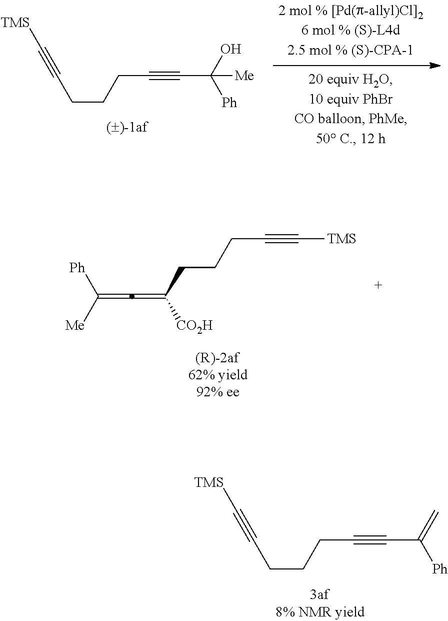

Operations were conducted by referring to Example 1. [Pd(π-allyl)Cl]₂ (0.0038 g, 0.01 mmol), chiral bisphosphine ligand (S)-L4d (0.0369 g, 0.03 mmol), (S)-CPA-1 (0.0102 g, 0.0125 mmol), (±)-1af (0.1421 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 15/1) to afford a product: chiral allenoic acid (R)-2af (0.0965 g, 62%): white solid; 92% ee (HPLC conditions: AS-H column, hexane/ⁱPrOH=98/2, 0.5 mL/min, λ=214 nm, t_{R}(minor)=13.3 min, t_{R}(major)=16.0 min); [α]_{D}²⁵=+5.5 (c=1.00, CHCl₃); melting point: 97.2-98.5° C. (petroleum ether/DCM); ¹H NMR (400 MHz, CDCl₃): δ=7.45-7.33 (m, 4 H, Ar—H), 7.33-7.26 (m, 1 H, Ar—H), 2.47 (td, J₁=7.5 Hz, J₂=2.3 Hz, 2 H, CH₂), 2.29 (t, J=7.0 Hz, 2 H, CH₂), 2.24 (s, 3 H, CH₃), 1.81-1.70 (m, 2 H, CH₂), 0.15 (s, 9 H, CH₃); ¹³C NMR (100 MHz, CDCl₃): δ=212.6, 172.6, 134.8, 128.6, 127.7, 126.1, 106.6, 105.6, 100.9, 84.9, 27.8, 26.9, 19.3, 16.4, 0.1; IR (neat): ν=2957, 2173, 1941, 1681, 1416, 1281, 1249, 1026 cm⁻¹; MS (70 eV, ESI) m/z: 335 (M+Nat), 313 (M+H⁺); Anal. Calcd. for C₁₉H₂₄O₂Si: C 73.03, H 7.74; found C 73.26, H 8.01.

### EXAMPLE 34

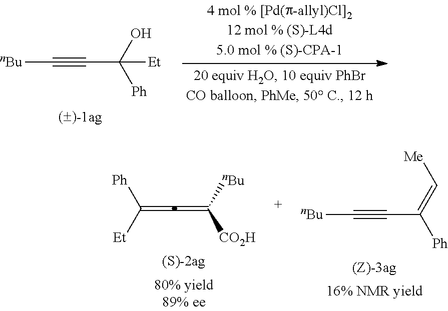

Operations were conducted by referring to Example 1. [Pd(π-allyl)Cl]₂ (0.0074 g, 0.02 mmol), chiral bisphosphine ligand (S)-L4d (0.0763 g, 0.06 mmol), (S)-CPA-1 (0.02 g, 0.025 mmol)), (±)-1ag (0.1085 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 15/1) to afford a product: chiral allenoic acid (S)-2ag (0.0975 g, 80%): white solid; 89% ee (HPLC conditions: AS-H column, hexane/ⁱPrOH=98/2, 1.0 mL/min, λ=214 nm, t_{R}(major)=7.4 min, t_{R}(minor)=9.1 min); [α]_{D}²⁵=+12.3 (c=1.00, CHCl₃); melting point: 64.4-65.4° C. (petroleum ether/DCM); ¹H NMR (400 MHz, CDCl₃): δ=7.38 (d, J=7.2 Hz, 2 H, Ar—H), 7.33 (t, J=7.8 Hz, 2 H, Ar—H), 7.27-7.21 (m, 1 H, Ar—H), 2.55 (quartet, J=7.3 Hz, 2 H, CH₂), 2.33 (t, J=7.6 Hz, 2 H, CH₂), 1.52-1.41 (m, 2 H, CH₂), 1.41-1.29 (m, 2 H, CH₂), 1.17 (t, J=7.4 Hz, 3 H, CH₃), 0.88 (t, J=7.2 Hz, 3 H, CH₃); ¹³C NMR (100 MHz, CDCl₃): δ=212.2, 173.3, 134.9, 128.6, 127.5, 126.3, 112.1, 103.8, 30.4, 28.4, 23.2, 22.4, 13.8, 12.3; IR (neat): ν=2960, 2931, 2873, 1939, 1678, 1415, 1277 cm⁻¹; MS (70 eV, EI) m/z (%): 245 (M⁺+1, 1.08), 244 (M⁺, 5.31), 129 (100); Anal. Calcd. for C₁₆H₂₀O₂: C 78.65, H 8.25; found C 78.73, H 8.40.

### EXAMPLE 35

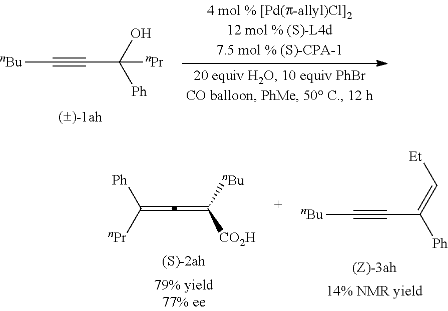

Operations were conducted by referring to Example 1. [Pd(π-allyl)Cl]₂ (0.0075 g, 0.02 mmol), chiral bisphosphine ligand (S)-L4d (0.0735 g, 0.06 mmol), (S)-CPA-1 (0.0302 g, 0.0375 mmol), (±)-1ag (0.1152 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 12 hours. Flash column chromatography (eluent: petroleum ether (60˜90° C.)/ethyl acetate=20/1, then 15/1) to afford a product: chiral allenoic acid (S)-2ag (0.1025 g, 79%): white solid; 77% ee (HPLC conditions: AS-H column, hexane/ⁱPrOH=98/2, 1.0 mL/min, λ=214 nm, t_{R}(major)=7.2 min, t_{R}(minor)=9.1 min); [α]_{D}²⁶=+12.7 (c=1.02, CHCl₃); melting point: 62.9-64.0° C. (petroleum ether/DCM); ¹H NMR (400 MHz, CDCl₃): δ=7.38 (d, J=6.8 Hz, 2 H, Ar—H), 7.33 (t, J=8.0 Hz, 2 H, Ar—H), 7.28-7.21 (m, 1 H, Ar—H), 2.51 (t, J=7.4 Hz, 2 H, CH₂), 2.32 (t, J=7.6 Hz, 2 H, CH₂), 1.64-1.51 (m, 2H, CH₂), 1.51-1.40 (m, 2 H, CH₂), 1.40-1.29 (m, 2 H, CH₂), 1.01 (t, J=7.4 Hz, 3 H, CH₃), 0.87 (t, J=7.2 Hz, 3 H, CH₃); ¹³C NMR (100 MHz, CDCl₃): δ=212.4, 173.2, 134.9, 128.6, 127.5, 126.4, 110.3, 102.9, 32.2, 30.4, 28.4, 22.4, 21.0, 13.9, 13.8; IR (neat): ν=2957, 2929, 2872, 1938, 1676, 1494, 1453, 1276 cm⁻¹; MS (70 eV, EI) m/z (%): 258 (M⁺, 6.49), 129 (100); Anal. Calcd. for C₁₇H₂₂O₂: C 79.03, H 8.58; found C 79.26, H 9.12.

### EXAMPLE 36

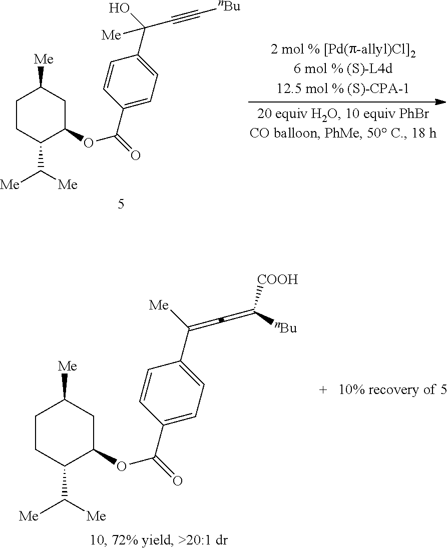

Operations were conducted by referring to Example 1. [Pd(π-allyl)Cl]₂ (0.0036 g, 0.01 mmol), chiral bisphosphine ligand (S)-L4d (0.0367 g, 0.03 mmol), (S)-CPA-1 (0.0502 g, 0.0625 mmol)), 5 (0.192 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1, then 10/1) to afford a product: chiral allenoic acid 10 (0.1492 g, 72%): oil substance; >20:1 dr; [α]_{D}²⁴=−37.7 (c=1.54, CHCl₃); ¹H NMR (400 MHz, CDCl₃): δ=8.02 (d, J=8.4 Hz, 2 H, Ar—H), 7.44 (d, J =8.4 Hz, 2 H, Ar—H), 4.92 (td, J₁=11.0 Hz, J₂=4.4 Hz, 1 H, CH), 2.34 (t, J=7.4 Hz, 2 H, CH₂), 2.21 (s, 3 H, CH₃), 2.17-2.09 (m, 1 H, CH), 2.02-1.86 (m, 1 H, CH), 1.73 (d, J=11.2 Hz, 2 H, CH₂), 1.64-1.50 (m, 2 H, CH₂), 1.50-1.40 (m, 2 H, CH₂), 1.40-1.30 (m, 2 H, CH₂), 1.20-1.02 (m, 2 H, CH₂), 1.00-0.84 (m, 10 H, CH and 3 x CH₃), 0.79 (d, J=7.2 Hz, 3 H, CH₃); ¹³C NMR (100 MHz, CDCl₃): δ=213.1, 172.3, 165.8, 139.7, 129.81, 129.76, 125.9, 104.8, 102.3, 74.9, 47.3, 40.9, 34.3, 31.4, 30.2, 28.2, 26.6, 23.7, 22.2, 22.0, 20.7, 16.6, 16.2, 13.8; IR (neat): ν=2956, 2928, 2868, 1941, 1709, 1683, 1271, 1112 cm⁻¹; MS (70 eV, ESI) m/z: 435 (M+Na⁺); HRMS calcd for C₂₆H₃₆O₄Na [M+Na⁺]: 435.2506, found: 435.2501.

### EXAMPLE 37

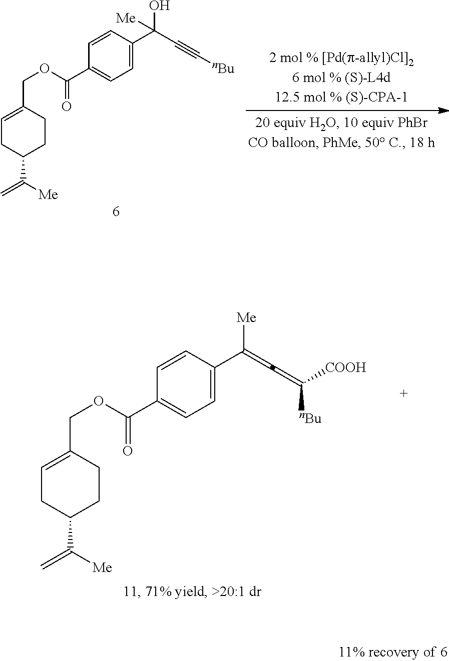

Operations were conducted by referring to Example 1. [Pd(π-allyl)Cl]₂ (0.0037 g, 0.01 mmol), chiral bisphosphine ligand (S)-L4d (0.0368 g, 0.03 mmol), (S)-CPA-1 (0.0504 g, 0.0625 mmol)), 6 (0.1902 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1, then 10/1) to afford a product: chiral allenoic acid 11 (0.1445 g, 71%): oil substance; >20:1 dr; [α]_{D}²⁵=−22.3 (c=1.00, CHCl₃); ¹H NMR (400 MHz, CDCl₃): δ=8.03 (d, J=8.4 Hz, 2 H, Ar—H), 7.44 (d, J=8.4 Hz, 2 H, Ar—H), 5.84 (s, 1 H, ═CH), 4.95-4.43 (m, 4 H, ═CH₂ and CH₂), 2.34 (t, J=7.4 Hz, 2 H, CH₂), 2.28-2.07 (m, 7 H, 2 x CH₂ and CH₃), 2.05-1.96 (m, 1 H, one proton of CH₂), 1.91-1.82 (m, 1 H, one proton of CH₂), 1.75 (s, 3 H, CH₃), 1.58-1.40 (m, 3 H, CH and CH₂), 1.39-1.29 (m, 2 H, CH₂), 0.88 (t, J=7.2 Hz, 3 H, CH₃); ¹³C NMR (100 MHz, CDCl₃): δ=213.1, 172.2, 166.2, 149.5, 139.9, 132.6, 129.9, 129.3, 125.9, 125.6, 108.8, 104.8, 102.3, 68.8, 40.8, 30.4, 30.1, 28.2, 27.3, 26.4, 22.2, 20.7, 16.2, 13.8; IR (neat): ν=2957, 2925, 2863, 1941, 1716, 1683, 1415, 1268, 1104 cm⁻¹; MS (70 eV, EI) m/z (%): 409 (M⁺+1, 1.37), 408 (M⁺, 4.44), 257 (100); HRMS calcd for C₂₆H₃₂O₄ [M⁺]: 408.2301, found: 408.2299.

### EXAMPLE 38

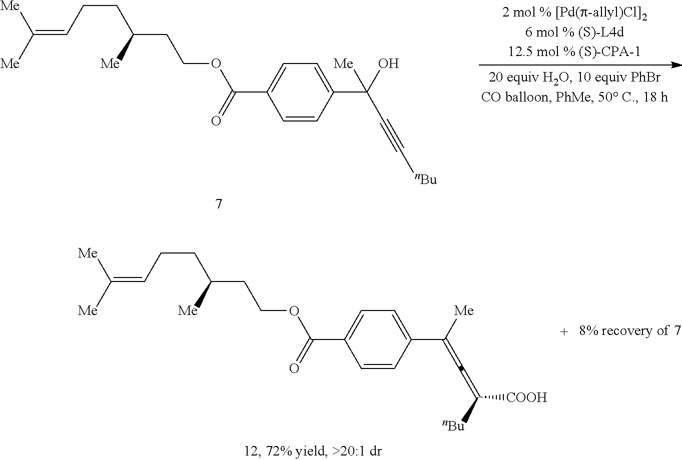

Operations were conducted by referring to Example 1. [Pd(π-allyl)Cl]₂ (0.0037 g, 0.01 mmol), chiral bisphosphine ligand (S)-L4d (0.0367 g, 0.03 mmol), (S)-CPA-1 (0.0506 g, 0.0625 mmol), 7 (0.1922 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1, then 10/1) to afford a product: chiral allenoic acid 12 (0.1488 g, 72%): oil substance; >20:1 dr; [α]_{D}²⁵=+11.2 (c=1.00, CHCl₃); ¹H NMR (400 MHz, CDCl₃): δ=8.01 (d, J=8.0 Hz, 2 H, Ar—H), 7.44 (d, J=8.4 Hz, 2 H, Ar—H), 5.10 (t, J=6.8 Hz, 1 H, ═CH), 4.45-4.23 (m, 2 H, CH₂), 2.34 (t, J=7.6 Hz, 2 H, CH₂), 2.21 (s, 3 H, CH₃), 2.09-1.92 (m, 2 H, CH₂), 1.86-1.77 (m, 1 H, CH), 1.72-1.52 (m, 8 H, CH₂ and 2 x CH₃), 1.50-1.21 (m, 6 H, 3 x CH₂), 0.97 (d, J=6.8 Hz, 3 H, CH₃), 0.88 (t, J=7.4 Hz, 3 H, CH₃); ¹³C NMR (100 MHz, CDCl₃): δ=213.1, 172.3, 166.4, 139.8, 131.3, 129.8, 129.4, 125.9, 124.5, 104.8, 102.3, 63.5, 36.9, 35.4, 30.1, 29.6, 28.2, 25.7, 25.4, 22.2, 19.5, 17.6, 16.2, 13.8; IR (neat): ν=2959, 2923, 2864, 1941, 1717, 1683, 1457, 1271, 1108 cm⁻¹; MS (70 eV, EI) m/z (%): 412 (M⁺, 2.99), 81 (100); HRMS calcd for C₂₆H₃₆O₄ [M⁺]: 412.2614, found: 412.2609.

### EXAMPLE 39

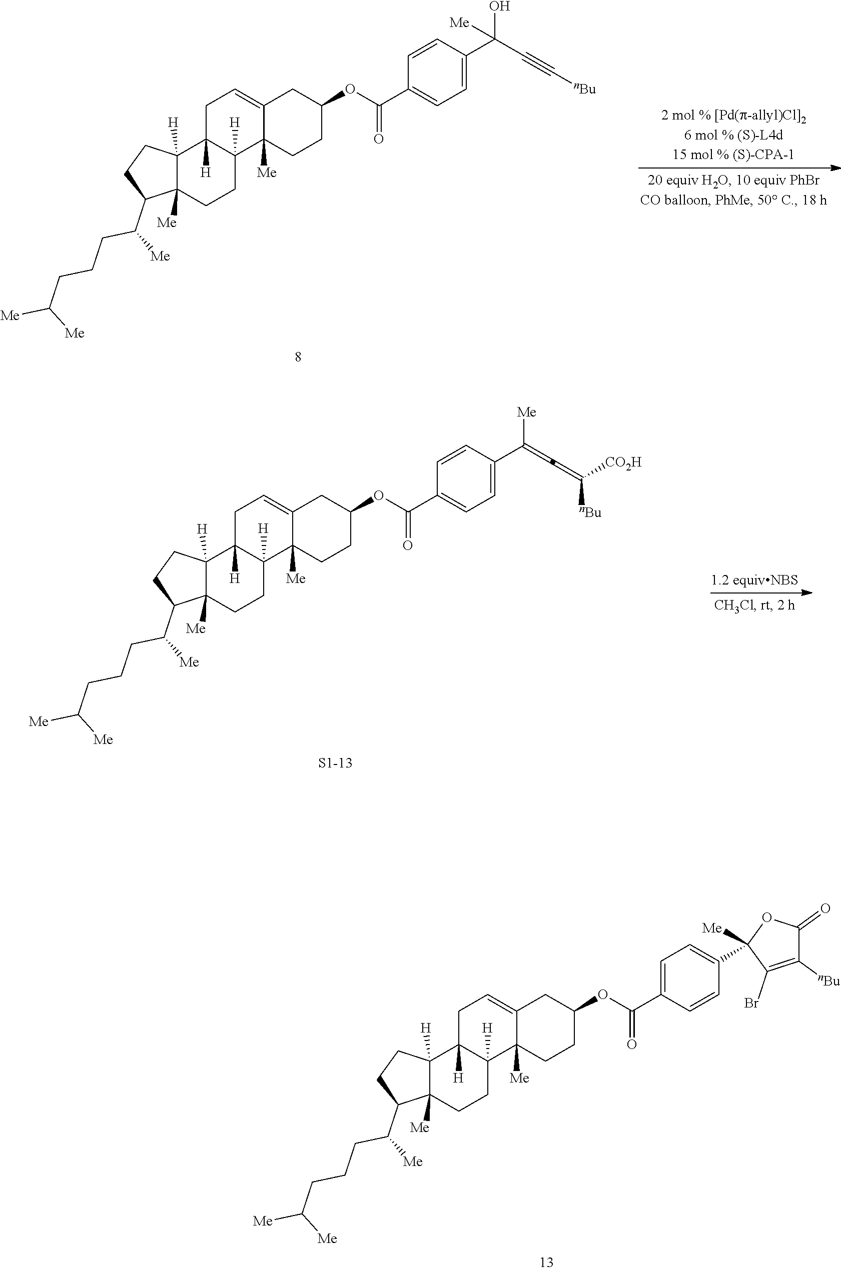

Operations were conducted by referring to Example 1. [Pd(π-allyl)Cl]₂ (0.0038 g, 0.01 mmol), chiral bisphosphine ligand (S)-L4d (0.0368 g, 0.03 mmol), (S)-CPA-1 (0.06 g, 0.0625 mmol)), 8 (0.3078 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=15/1, then 10/1) to afford a crude product: chiral allenoic acid S1-13 (0.2963 g), all of which was put to the next reaction.

S1-13 (0.2963 g, ˜0.5 mmol), NBS (N-bromosuccinimide) (0.1064 g, 0.6 mmol) and CHCl₃ (5 mL) were added in sequence to a dry Schlenk reaction tube. The reaction tube was plugged with a rubber stopper, then reacted at room temperature for 2 hours, concentrated, and subjected to flash column chromatography (eluent: petroleum ether (60˜90° C.)/ethyl acetate=15/1, then 10/1) to afford a product: bromo chiral γ-butyrolactone 13 (0.2603 g, 72%): oil substance; >20:1 dr; [α]_{D}²³=+63.5 (c=1.00, CHCl₃); melting point: 183.3-184.2° C. (petroleum ether/DCM); ¹H NMR (400 MHz, CDCl₃): δ=8.05 (d, J=8.4 Hz, 2 H, Ar—H), 7.45 (d, J=8.4 Hz, 2 H, Ar—H), 5.48-5.32 (m, 1 H, ═CH), 4.95-4.74 (m, 1 H, CH), 2.45 (d, J=7.6 Hz, 2 H, CH₂), 2.36 (t, J=7.6 Hz, 2 H, CH₂), 2.06-1.67 (m, 9 H), 1.64-1.42 (m, 8 H), 1.40-1.29 (m, 5 H), 1.29-0.96 (m, 14 H), 0.95-0.89 (m, 6 H), 0.87 (dd, J₁=6.8 Hz, J₂=1.6 Hz, 6 H, 2 x CH₃), 0.69 (s, 3 H, CH₃); ¹³C NMR (100 MHz, CDCl₃): δ=170.1, 165.2, 149.8, 141.9, 139.4, 131.6, 131.3, 129.8, 125.5, 122.8, 87.7, 74.8, 56.6, 56.1, 50.0, 42.2, 39.7, 39.4, 38.1, 36.9, 36.6, 36.1, 35.7, 31.9, 31.8, 28.9, 28.2, 27.9, 27.8, 24.8, 24.2, 23.9, 23.8, 22.8, 22.5, 22.3, 21.0, 19.3, 18.7, 13.7, 11.8; IR (neat): ν=2939, 2861, 1749, 1717, 1461, 1274, 1111, 1025 cm⁻¹; MS (DART) m/z: 740 (M(⁸¹Br)+NH₄⁺); 738 (M(⁷⁹Br)+NH₄⁺; Anal. Calcd. for C₄₃H₆₁BrO₄: C 71.55, H 8.52; found C 71.42, H 8.71.

### EXAMPLE 40

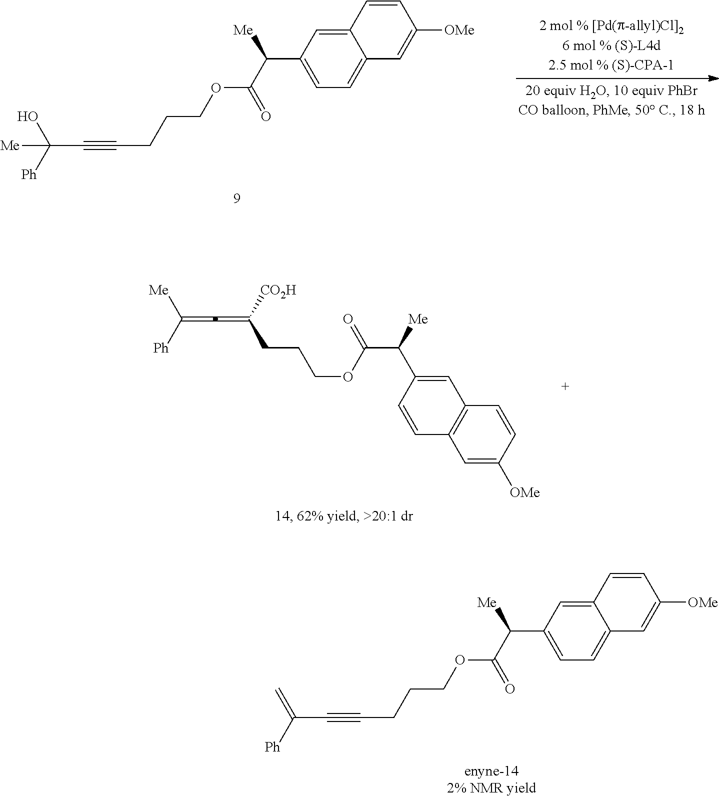

Operations were conducted by referring to Example 1. [Pd(π-allyl)Cl]₂ (0.0037 g, 0.01 mmol), chiral bisphosphine ligand (S)-L4d (0.0364 g, 0.03 mmol), (S)-CPA-1 (0.0101 g, 0.0125 mmol)), 9 (0.2087 g, 0.5 mmol), bromobenzene (527 μL, d=1.49 g/mL, 0.7860 g, 5 mmol), water (180 μL, d=1.0 g/mL, 0.18 g, 10 mmol), toluene (2 mL) were reacted at 50° C. for 18 hours. Flash column chromatography (eluent: petroleum ether (60˜90° C.)/ethyl acetate=10/1, then petroleum ether (60˜90° C.)/ether/dichloromethane=4/1/1) to afford a product: chiral allenoic acid 14 (0.1379 g, 62%): oil substance; >20:1 dr; [α]_{D}²¹=+15.7 (c=1.10, CHCl₃); ¹H NMR (400 MHz, CDCl₃): δ=7.70-7.54 (m, 3 H, Ar—H), 7.36 (dd, J₁=8.6 Hz, J₂=1.4 Hz, 1 H, Ar—H), 7.33-7.21 (m, 5 H, Ar—H), 7.09 (dd, J₁=9.0 Hz, J₂=2.6 Hz, 1 H, Ar—H), 7.07-7.03 (m, 1 H, Ar—H), 4.15-4.02 (m, 2 H, CH₂), 3.90-3.75 (m, 4 H, CH and OCH₃), 2.39-2.21 (m, 2 H, CH₂), 2.11 (s, 3 H, CH₃), 1.82-1.70 (m, 2 H, CH₂), 1.54 (d, J=7.2 Hz, 3 H, CH₃); ¹³C NMR (100 MHz, CDCl₃): δ=212.4, 174.6, 172.2, 157.5, 135.6, 134.6, 133.6, 129.2, 128.8, 128.5, 127.7, 127.0, 126.1, 126.0, 125.8, 118.8, 105.7, 105.5, 100.6, 63.8, 55.2, 45.3, 26.9, 25.1, 18.3, 16.2; IR (neat): ν=2938, 2850, 1941, 1725, 1682, 1454, 1265, 1182, 1029 cm⁻¹; MS (70 eV, ESI) m/z: 467 (M+Na⁺); HRMS calcd for C₂₈H₂₈O₅Na [M+Na⁺]: 467.1829, found: 467.1826.

### EXAMPLE 41

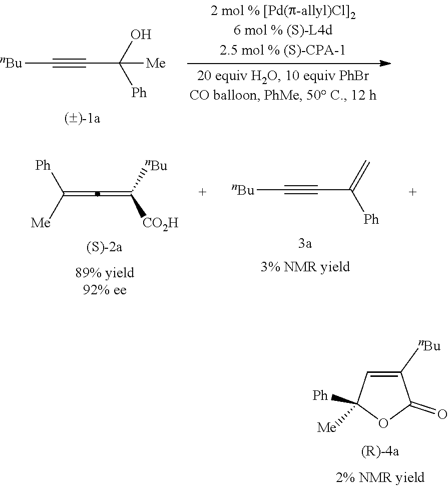

Operations were conducted by referring to Example 1. [Pd(π-allyl)Cl]₂ (0.0368 g, 0.1 mmol), chiral bisphosphine ligand (S)-L4d (0.3638 g, 0.3 mmol), (S)-CPA-1 (0.1009 g, 0.0125 mmol), (±)-1a (1.0109 g, 5.0 mmol), bromobenzene (5.27 mL, d=1.49 g/mL, 7.8523 g, 50 mmol), water (1.8019 g, 100 mmol), toluene (20 mL) were reacted at 50° C. for 12 hours. Flash column chromatography (eluent: petroleum ether (60˜90° C.)/ethyl acetate=15/1, then 10/1) to afford a product: chiral allenoic acid (S)-2a (1.0227 g, 89%): white solid; 92% ee (HPLC conditions: AS-H column, hexane/ⁱPrOH=98/2, 1.0 mL/min, λ=214 nm, t_{R}(major)=8.5 min, t_{R}(minor)=11.9 min); ¹H NMR (400 MHz, CDCl₃): δ=7.44-7.29 (m, 4 H, Ar—H), 7.28-7.21 (m, 1 H, Ar—H), 2.32 (t, J=7.4 Hz, 2 H, CH₂), 2.19 (s, 3 H, CH₃), 1.52-1.41 (m, 2 H, CH₂), 1.40-1.28 (m, 2 H, CH₂), 0.88 (t, J=7.2 Hz, 3 H, CH₃); ¹³C NMR (100 MHz, CDCl₃): δ=212.6, 172.8, 135.0, 128.5, 127.6, 126.1, 105.2, 101.8, 30.2, 28.3, 22.2, 16.3, 13.8.

### EXAMPLE 42

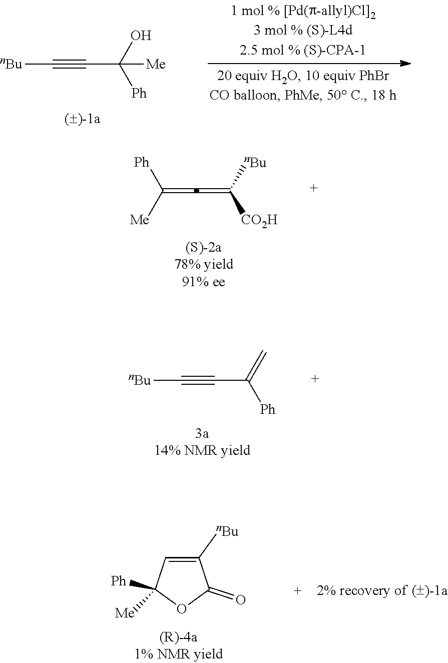

Operations were conducted by referring to Example 1. [Pd(π-allyl)Cl]₂ (0.0184 g, 0.1 mmol), chiral bisphosphine ligand (S)-L4d (0.1818 g, 0.3 mmol), (S)-CPA-1 (0.1007 g, 0.0125 mmol), (±)-1a (1.0115 g, 5.0 mmol), bromobenzene (5.27 mL, d=1.49 g/mL, 7.8523 g, 50 mmol), water (1.8008 g, 100 mmol), toluene (20 mL) were reacted at 50° C. for 18 hours. Flash column chromatography (eluent: petroleum ether (60˜90° C.)/ethyl acetate=20/1, then 15/1) to afford a product: chiral allenoic acid (S)-2a (0.8994 g, 78%): white solid; 91% ee (HPLC conditions: AS-H column, hexane/ⁱPrOH=98/2, 1.0 mL/min, λ=214 nm, t_{R}(major)=8.7 min, t_{R}(minor)=12.3 min); ¹H NMR (400 MHz, CDCl₃): δ=7.43-7.28 (m, 4 H, Ar—H), 7.27-7.22 (m, 1 H, Ar—H), 2.32 (t, J=7.6 Hz, 2 H, CH₂), 2.19 (s, 3 H, CH₃), 1.52-1.41 (m, 2 H, CH₂), 1.40-1.28 (m, 2 H, CH₂), 0.88 (t, J=7.2 Hz, 3 H, CH₃); ¹³C NMR (100 MHz, CDCl₃): δ=212.5, 172.5, 135.0, 128.5, 127.6, 126.1, 105.2, 101.8, 30.2, 28.3, 22.3, 16.3, 13.8.

### EXAMPLE 43

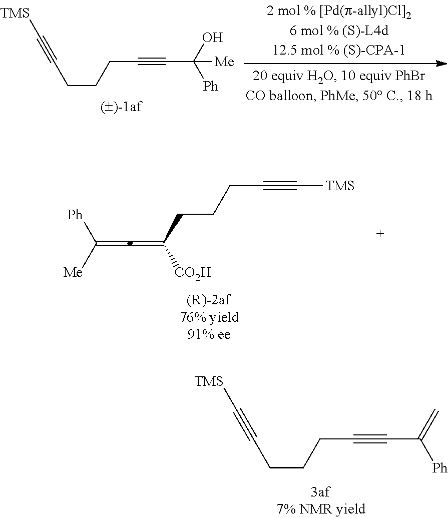

Operations were conducted by referring to Example 1. [Pd(π-allyl)Cl]₂ (0.0367 g, 0.1 mmol), chiral bisphosphine ligand (S)-L4d (0.3634 g, 0.3 mmol), (S)-CPA-1 (0.1008 g, 0.0125 mmol), (±)-1af (1.4219 g, 5.0 mmol), bromobenzene (5.27 mL, d=1.49 g/mL, 7.8523 g, 50 mmol), water (1.8012 g, 100 mmol), toluene (20 mL) were reacted at 50° C. for 18 hours. Flash column chromatography (eluent: petroleum ether (60˜90° C.)/ethyl acetate=20/1, then 15/1) to afford a product: chiral allenoic acid (R)-2af (1.1935 g, 76%): white solid; 91% ee (HPLC conditions: AS-H column, hexane/ⁱPrOH=98/2, 1.0 mL/min, λ=214 nm, t_{R}(minor)=6.7 min, t_{R}(major)=8.1 min); ¹H NMR (400 MHz, CDCl₃): δ=7.42-7.32 (m, 4 H, Ar—H), 7.28-7.23 (m, 1 H, Ar—H), 2.43 (td, J₁=7.5 Hz, J₂=2.3 Hz, 2 H, CH₂), 2.26 (t, J=7.0 Hz, 2 H, CH₂), 2.21 (s, 3 H, CH₃), 1.76-1.65 (m, 2 H, CH₂), 0.12 (s, 9 H, CH₃); ¹³C NMR (100 MHz, CDCl₃): δ=212.6, 172.6, 134.8, 128.6, 127.7, 126.1, 106.6, 105.5, 100.9, 84.9, 27.8, 26.9, 19.3, 16.4, 0.1.

### EXAMPLE 44

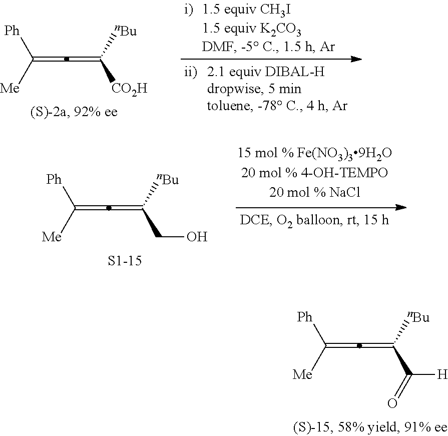

(S)-2a (0.4608 g, 2 mmol, 92% ee), K₂CO₃ (0.4152 g, 3 mmol), DMF (N,N-dimethylformamide) (10 mL) was added in sequence to a dry Schlenk reaction tube, the reaction tube was placed in a −5° C. cold bath, and then CH₃I (iodomethane) (188 μL, d=2.28 g/mL, 0.4286 g, 3 mmol) was added, the reaction was completed after stirred in a −5° C. cold bath for 1.5 hours as monitored by thin layer chromatography (TLC). The reaction was quenched by water (10 mL), the aqueous phase was extracted with ether (10 mL×3), the organic phases were combined, washed once with saturated ammonium chloride solution (10 mL), once with saturated brine (10 mL), separated and dried over anhydrous sodium sulfate, filtered and concentrated to afford an oily chiral allenoate which was used directly in the next reaction. All the S1 and toluene (10 mL) obtained in the previous step were added to a dry Schlenk reaction tube, the reaction tube was placed in a −78° C. cold bath and added with DIBAL-H (diisobutylaluminum hydride) (4.2 mL, 1.0 M in Hexane, 4.2 mmol) dropwise, the reaction was completed after stirred in a −5° C. cold bath for 4 hours as monitored by thin layer chromatography (TLC). The reaction was quenched by methanol (10 mL) at −78° C., the reaction tube was taken out of the cold bath, and after returning to room temperature, added with water (20 mL) and 1 mol/L aqueous hydrochloric acid solution (20 mL), the aqueous phase was extracted with ether (10 mL×3), the organic phases were combined, washed once with saturated brine (10 mL), separated, and dried over anhydrous sodium sulfate, filtered, concentrated, and subjected to flash silica gel column chromatography (eluent: petroleum ether (60˜90° C.)/ethyl acetate=20/1) to afford chiral allenol S1-15, which is directly used in the next reaction.

All the S1-15 obtained in the previous step, Fe(NO₃)₃·9H₂O (0.121 g, 0.3 mmol), 4-OH-TEMPO (0.0687 g, 0.4 mmol), NaCl (0.0236 g, 0.4 mmol), and DCE (1,2-dichloroethane) (8 mL) were added to a dry Schlenk reaction tube, the reaction was completed after stirred at room temperature for 15 hours as monitored by thin layer chromatography (TLC), and the reaction solution was filtered through a short silica gel column (3 cm), then subjected to flash column chromatography (eluent: petroleum ether (60˜90° C.)/ether/dichloromethane=100/1/1) to afford a product: chiral biuronic acid 15 (0.2478 g, 58%): oil substance; 91% ee (HPLC conditions: AS-H column, hexanePPrOH=99/1, 1.0 mL/min, λ=214 nm, t_{R}(minor)=6.5 min, t_{R}(major)=7.6 min); [α]_{D}²³=−5.1 (c=1.02, CHCl₃); oil; ¹H NMR (400 MHz, CDCl₃): δ=9.60 (s, 1 H, CHO), 7.44-7.33 (m, 4 H, Ar—H), 7.32-7.26 (m, 1 H, Ar—H), 2.31 (t, J=7.6 Hz, 2 H, CH₂), 2.26 (s, 3 H, CH₃), 1.52-1.42 (m, 2 H, CH₂), 1.42-1.32 (m, 2 H, CH₂), 0.90 (t, J=7.2 Hz, 3 H, CH₃); ¹³C NMR (100 MHz, CDCl₃): δ=219.5, 192.1, 134.5, 128.7, 127.9, 125.9, 113.5, 106.6, 29.9, 24.8, 22.4, 16.6, 13.8; IR (neat): ν=2960, 2866, 1931, 1680, 1452, 1171 cm⁻¹; MS (70 eV, EI) m/z (%): 215 (M⁺+1, 3.12), 214 (M⁺, 5.61), 128 (100); HRMS calcd for C₁₅H₁₈O [M⁺]: 214.1352, found: 214.1355.

### EXAMPLE 45

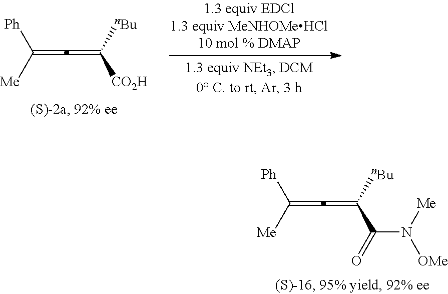

(S)-2a (0.1151 g, 0.5 mmol, 92% ee), 1-(3-dimethylaminopropyl)-3-ethylcarbodiimide hydrochloride (EDCI) (0.1245 g, 0.65 mmol), dimethylhydroxylamine hydrochloride (0.0637 g, 0.65 mmol), 4-dimethylaminopyridine (DMAP) (0.0063 g, 0.05 mmol), triethylamine (NEt₃) (90 μL, d=0.728 g/mL, 0.0655 g, 0.65 mmol) were added to a dry Schlenk reaction tube, which was replaced with argon three times, then dichloromethane (DCM) (2 mL) was added, and the reaction was completed after stirred in a cold bath at 0° C. for 3 hours as monitored by thin layer chromatography (TLC). After diluted by Dichloromethane (5 mL), the reaction was quenched by water (5 mL), the aqueous phase was extracted with dichloromethane (5 mL×3), the organic phases were combined, washed once with saturated brine (5 mL), separated and dried over anhydrous sodium sulfate, filtered, concentrated, and subjected to flash silica gel column chromatography (eluent: petroleum ether (60˜90° C.)/ethyl acetate=10/1) to afford a product: allenamide (S)-16 (0.1296 g, 95%): oil substance; 92% ee (HPLC conditions: AS-H column, hexane/ⁱPrOH=98/2, 1.0 mL/min, λ=214 nm, t_{R}(major)=6.5 min, t_{R}(minor)=7.8 min); [α]_{D}²⁰=+125.0 (c=1.00, CHCl₃); ¹H NMR (400 MHz, CDCl₃): δ=7.41 (d, J=8.8 Hz, 2 H, Ar—H), 7.34 (t, J=7.6 Hz, 2 H, Ar—H), 7.25-7.19 (m, 1 H, Ar—H), 3.51 (s, 3 H, CH₃), 3.22 (s, 3 H, CH₃), 2.41 (t, J=7.4 Hz, 2 H, CH₂), 2.17 (s, 3 H, CH₃), 1.51-1.42 (m, 2 H, CH₂), 1.42-1.31 (m, 2 H, CH₂), 0.89 (t, J=7.2 Hz, 3 H, CH₃); ¹³C NMR (100 MHz, CDCl₃): δ=206.3, 168.1, 136.0, 128.4, 127.0, 125.8, 102.1, 101.0, 61.1, 33.9, 30.2, 30.1, 22.4, 16.5, 13.9; IR (neat): ν=2956, 2928, 2864, 1942, 1637, 1453, 1365, 1186 cm⁻¹; MS (70 eV, ESI) m/z: 296 (M+Na⁺), 274 (M+H⁺); HRMS calcd for C₁₇H₂₃O₂N [M+H⁺]: 274.1802, found: 274.1800.

### EXAMPLE 46

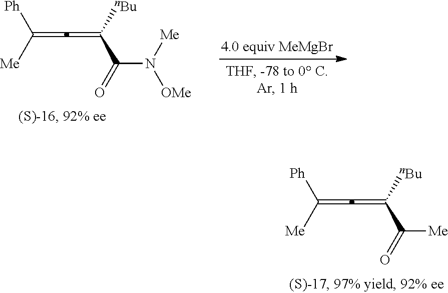

(S)-16 (0.0545 g, 0.2 mmol, 92% ee), and tetrahydrofuran (THF) (1 mL) were added to a dry Schlenk reaction tube, which was replaced with argon three times, then the reaction tube was put in −78° C. cold bath, and methylmagnesium bromide (0.27 mL, 3.0 M in hexane, 0.81 mmol) was added. Then the reaction was completed after stirred in a cold bath at 0° C. for 1 hour as monitored by thin layer chromatography (TLC). The reaction was quenched by saturated ammonium chloride (1 mL)at 0° C., the aqueous phase was extracted with ethyl acetate (2 mL×3), the organic phases were combined, washed once with saturated brine (3 mL), separated and dried over anhydrous sodium sulfate, filtered, concentrated, and subjected to flash silica gel column chromatography (eluent: petroleum ether (60˜90° C.)/ethyl acetate=20/1) to afford a product: allenone (S)-17 (0.044 g, 97%): oil substance; 92% ee (HPLC conditions: AD-H column, hexane/ⁱPrOH=99.5/0.5, 0.5 mL/min, λ=214 nm, t_{R}(minor)=11.8 min, t_{R}(major)=12.3 min); [α]_{D}²¹=+58.6 (c=1.01, CHCl₃); ¹H NMR (400 MHz, CDCl₃): δ=7.45-7.31 (m, 4 H, Ar—H), 7.30-7.24 (m, 1 H, Ar—H), 2.31 (t, J=7.4 Hz, 2 H, CH₂), 2.26 (s, 3 H, CH₃), 2.23 (s, 3 H, CH₃), 1.46-1.30 (m, 4 H, CH₂), 0.88 (t, J=7.2 Hz, 3 H, CH₃); ¹³C NMR (100 MHz, CDCl₃): δ=213.8, 198.9, 134.9, 128.7, 127.5, 125.7, 111.4, 104.8, 30.1, 27.2, 26.7, 22.4, 16.4, 13.8; IR (neat): ν=2955, 2925, 2860, 1931, 1676, 1454, 1358, 1234 cm⁻¹; MS (70 eV, EI) m/z (%): 229 (M⁺+1, 1.53), 228 (M⁺, 8.77), 185 (100); HRMS calcd for C₁₆H₂₀O [M⁺]: 228.1509, found: 228.1509.

### EXAMPLE 47

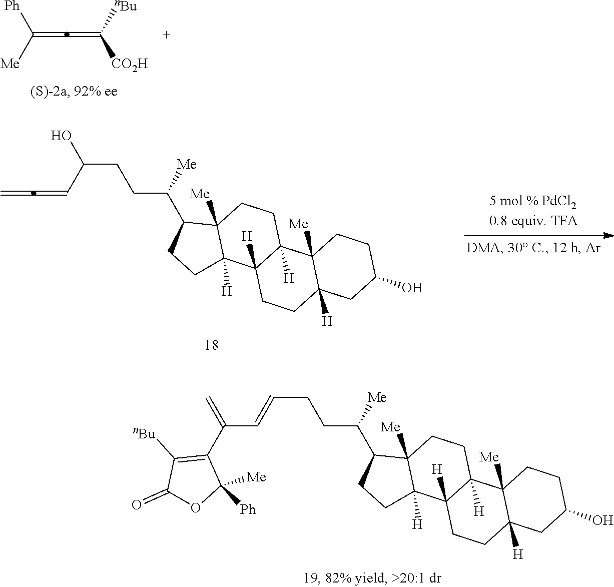

(S)-2a (0.0462 g, 0.2 mmol, 92% ee), 18 (0.1119 g, 0.28 mmol), and PdCl₂ (0.0019 g, 0.01 mmol) were added to a dry Schlenk reaction tube, which was replaced with argon three times, then TFA (trifluoroacetic acid) (12 uL, d=1.535 g/mL, 0.0184 g, 0.16 mmol) and DMA (N,N-dimethylacetamide) (2.5 mL) were added, and the reaction tube was placed in an oil bath that had been preheated to 30° C., the reaction was completed after stirred for 12 hours as monitored by thin layer chromatography (TLC). The reaction was quenched by water (2.5 mL), the aqueous phase was extracted with ether (3 mL×3), the organic phases were combined, washed once with saturated brine (5 mL), separated and dried over anhydrous sodium sulfate, filtered, concentrated, and subjected to flash silica gel column chromatography (eluent: petroleum ether (60˜90° C.)/ether/dichloromethane=10/1/1) to afford a chiral cyclic product 19 (0.1009 g, 82%): Oil substance; >20:1 dr; [α]_{D}²⁴=+81.2 (c=1.12, CHCl₃); ¹H NMR (400 MHz, CDCl₃): δ=7.33-7.20 (m, 5 H, Ar—H), 5.93 (d, J=15.6 Hz, 1 H, ═CH), 5.20-4.98 (m, 2 H, 2 x ═CH), 4.36 (s, 1 H, ═CH), 3.70-3.55 (m, 1 H, CH), 2.15 (td, J₁=7.8 Hz, J₂=2.1 Hz, 2 H, CH₂), 2.00-1.89 (m, 3 H), 1.87-1.73 (m, 7 H), 1.69-1.62 (m, 1 H), 1.61-1.45 (m, 4 H), 1.41-0.94 (m, 20 H), 0.94-0.81 (m, 10 H), 0.61 (s, 3 H, CH₃); ¹³C NMR (100 MHz, CDCl₃): δ=173.4, 164.6, 138.63, 138.58, 135.4, 128.8, 128.2, 128.1, 128.0, 125.3, 116.9, 88.0, 71.7, 56.4, 56.0, 42.6, 42.0, 40.3, 40.1, 36.3, 35.7, 35.3, 35.1, 34.5, 30.4, 29.8, 29.2, 28.2, 27.1, 26.3, 24.2, 24.14, 24.09, 23.3, 22.5, 20.7, 18.3, 13.7, 11.9; IR (neat): ν=3351, 2932, 2862, 2173, 1754, 1449, 1221, 1040 cm⁻¹; MS (70 eV, ESI) m/z: 635 (M+Na⁺), 613 (M+H⁺); HRMS calcd for C₄₂H₆₁O₃ [M+H⁺]: 613.4615, found: 613.4612.

### EXAMPLE 48

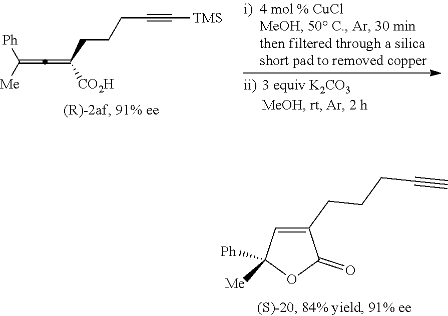

(R)-2af (0.6242 g, 2.0 mmol, 91% ee), CuCl (0.008 g, 0.08 mmol, weighed in a glove box) were added to a dry Schlenk reaction tube, which was replaced with argon three times, then MeOH (10 mL) was added, and the reaction tube was placed in an oil bath that had been preheated to 50° C., the reaction was completed after stirred for 30 minutes as monitored by thin layer chromatography (TLC). The resulting mixture was quickly filtered through a short silica gel column (3 cm) to remove the copper salt, eluted by 30 mL of ethyl acetate, and spin-dried to afford an oily substance, which was directly used in the next reaction. The above oil substance, K₂CO₃ (0.8291 g, 6 mmol) were added to a dry Schlenk reaction tube, which was replaced with argon three times, then MeOH (10 mL) was added, the reaction was completed after stirred at room temperature for 2 hours as monitored by thin layer chromatography (TLC), filtered, concentrated, and subjected to flash silica gel column chromatography (eluent: petroleum ether (60˜90° C.)/ether/dichloromethane=20/1/1) to afford a chiral cyclic product (S)-20 (0.4042 g, 84%): oil substance; 91% ee (HPLC conditions: AD-H column, hexane/ⁱPrOH=99/1, 0.9 mL/min, λ=214 nm, t_{R}(minor)=32.5 min, t_{R}(major)=36.4 min); [α]_{D}²³=−103.3 (c=1.07, CHCl₃); ¹H NMR (400 MHz, CDCl₃): δ=7.44-7.22 (m, 6 H, Ar—H), 2.43 (t, J=7.6 Hz, 2 H, CH₂), 2.23 (td, J₁=6.9 Hz, J₂=2.5 Hz, 2 H, CH₂), 1.97 (t, J=2.6 Hz, 1 H, CH), 1.87-1.70 (m, 5 H, CH₂ and CH₃); ¹³C NMR (100 MHz, CDCl₃): δ=172.9, 152.9, 140.0, 131.3, 128.7, 128.1, 124.7, 86.7, 83.3, 69.2, 26.8, 25.9, 24. 1, 17.9; IR (neat): ν=3294, 2933, 2116, 1750, 1444, 1261, 1036 cm⁻¹; MS (70 eV, ESI) m/z: 263 (M+Na⁺), 241 (M+El+); HRMS calcd for C₁₆H₁₇O₂ a [M+H⁺]: 241.1223, found: 241.1222.

### EXAMPLE 49

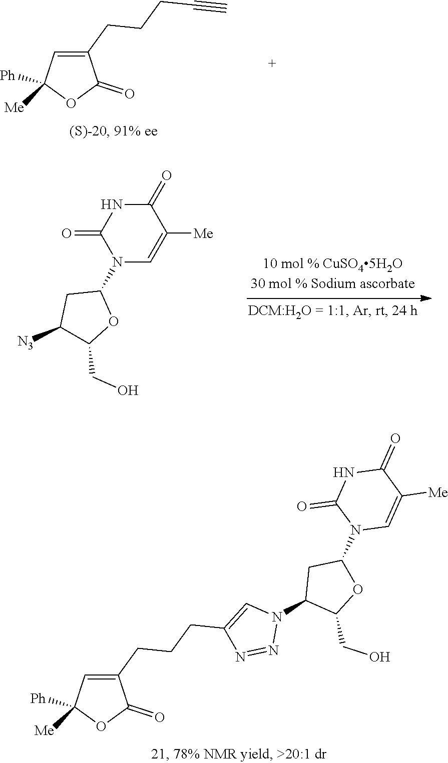 [SMILES: C#CCCCC1=C[C@@](C)(c2ccccc2)OC1=O ; Cc1cn([C@H]2C[C@H](N=[N+]=[N-])[C@@H](CO)O2)c(=O)[nH]c1=O ; Cc1cn([C@H]2C[C@H](n3cc(CCCC4=C[C@@](C)(c5ccccc5)OC4=O)nn3)[C@@H](CO)O2)c(=O)[nH]c1=O]

Zidovudine (0.0681 g, 0.24 mmol), chiral cyclic product (S)-20 (0.0483 g, 0.2 mmol, 91% ee) were added to a dry Schlenk reaction tube, which replaced with argon three times, then DCM (1 mL), aqueous sodium ascorbate (0.012 g, 0.06 mmol, dissolved in 0.5 mL water), aqueous CuSO₄·5H₂O (0.005 g, 0.02 mmol, dissolved in 0.5 mL water) were added, and the reaction was completed after stirred at room temperature for 24 hours as monitored by thin layer chromatography (TLC). After diluted by DCM (5 mL), the reaction solution was washed with saturated brine (5 mL), separated and dried over anhydrous sodium sulfate, filtered, concentrated, and subjected to flash silica gel column chromatography (eluent: ethyl acetate elution, then dichloromethane/methanol=10/1) to afford product 21 (0.0793 g, 78%): oil substance; >20:1 dr; [α]_{D}²²=−62.3 (c=1.19, CHCl₃); ¹H NMR (400 MHz, CDCl₃): δ=10.0-9.71 (m, 1 H, NH), 7.68-7.46 (m, 2 H, 2 x ═CH), 7.41-7.18 (m, 6 H, ═CH and Ar—H), 6.30 (t, J=6.4 Hz, 1 H, CH), 5.55-5.32 (m, 1 H, CH), 4.46-4.32 (m, 1 H, CH), 4.22 (br, 1 H, OH), 4.00 (d, J=11.6 Hz, 1 H, one proton of CH₂), 3.81 (d, J=11.2 Hz, 1 H, one proton of CH₂), 3.03-2.89 (m, 2 H, CH₂), 2.75 (t, J=7.2 Hz, 2 H, CH₂), 2.39-2.26 (m, 2 H, CH₂), 2.01-1.89 (m, 2 H, CH₂), 1.87 (s, 3 H, CH₃), 1.77 (s, 3 H, CH₃); ¹³C NMR (100 MHz, CDCl₃): δ=173.2, 164.3, 153.2, 150.6, 147.4, 139.8, 137.5, 131.4, 128.7, 128.1, 124.6, 121.2, 110.9, 87.1, 86.9, 85.1, 61.3, 59.1, 37.7, 26.9, 26.7, 24.8, 24.4, 12.3; IR (neat): ν=3454, 2932, 2249, 1748, 1684, 1463, 1267, 1101, 1051 cm⁻¹; MS (70 eV, ESI) m/z: 508 (M+El+); HRMS calcd for C₂₆H₃₀O₆N₅ [M+Na⁺]: 508.2191, found: 508.2190.

### EXAMPLE 50

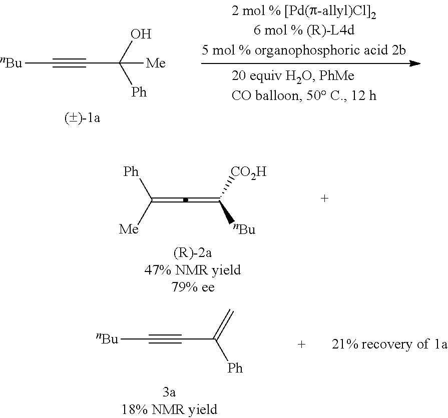

Operations were conducted by referring to Example 1. [Pd(π-allyl)Cl]₂ (0.0015 g, 0.004 mmol), chiral bisphosphine ligand (R)-L4d (0.0147 g, 0.0012 mmol), organophosphoric acid 2b (0.0054 g, 0.01 mmol), (±)-1a (0.0402 g, 0.2 mmol), water (72 μL, d=1.0 g/mL, 0.072 g, 4 mmol), toluene (1.0 mL) were reacted at 50° C. for 12 hours, purified by preparative plate chromatography (developing solvent: petroleum ether (60˜90° C.)/ethyl acetate=5/1) to affored a product: chiral allenoic acid (R)-2a (NMR yield 47%): 79% ee (HPLC conditions: AS-H column, hexane/ⁱPrOH=98/2, 1.0 mL/min, λ=214 nm, t_{R}(major)=8.7 min, t_{R}(minor)=11.2 min).

### EXAMPLE 51

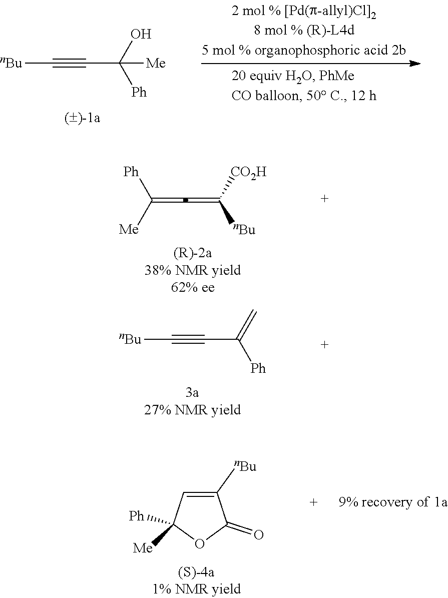

Operations were conducted by referring to Example 1. [Pd(π-allyl)Cl]₂ (0.0015 g, 0.004 mmol), chiral bisphosphine ligand (R)-L4d (0.0147 g, 0.0012 mmol), organophosphoric acid 2b (0.0053 g, 0.01 mmol), (±)-1a (0.0399 g, 0.2 mmol), water (72 μL, d=1.0 g/mL, 0.072 g, 4 mmol), fluorobenzene (1.0 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 38%): 62% ee (HPLC conditions: AS-H column, hexane/ⁱPrOH=98/2, 1.0 mL/min, λ=214 nm, t_{R}(major)=8.2 min, t_{R}(minor)=10.4 min).

### EXAMPLE 52

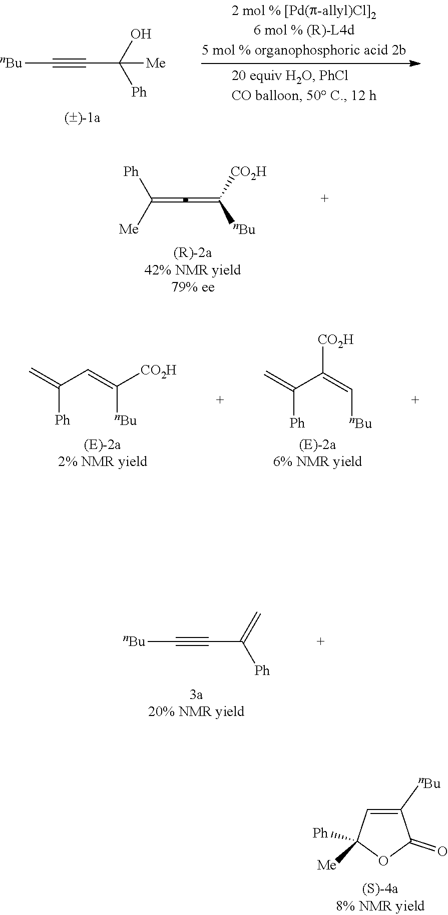

Operations were conducted by referring to Example 1. [Pd(π-allyl)Cl]₂ (0.0014 g, 0.004 mmol), chiral bisphosphine ligand (R)-L4d (0.0148 g, 0.0012 mmol), organophosphoric acid 2b (0.0052 g, 0.01 mmol), (±)-1a (0.0402 g, 0.2 mmol), water (72 d=1.0 g/mL, 0.072 g, 4 mmol), chlorobenzene (1.0 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 42%): 68% ee (HPLC conditions: AS-H column, hexane/ⁱPrOH=98/2, 1.0 mL/min, λ=214 nm, t_{R}(major)=8.3 min, t_{R}(minor)=10.5 min).

### EXAMPLE 53

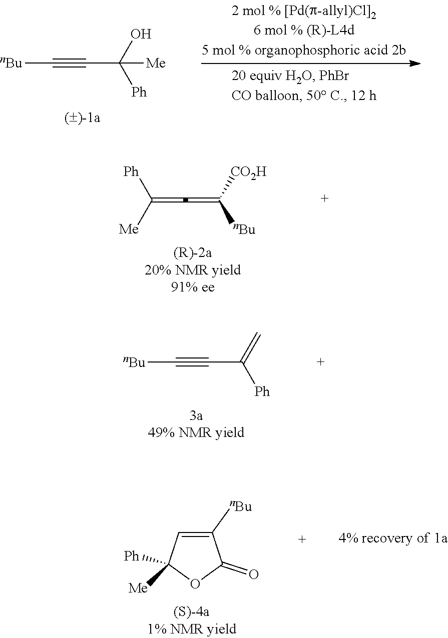

Operations were conducted by referring to Example 1. [Pd(π-allyl)Cl]₂ (0.0016 g, 0.004 mmol), chiral bisphosphine ligand (R)-L4d (0.0147 g, 0.0012 mmol), organophosphoric acid 2b (0.0052 g, 0.01 mmol), (±)-1a (0.0403 g, 0.2 mmol), water (72 d=1.0 g/mL, 0.072 g, 4 mmol), bromobenzene (1.0 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 30%): 91% ee (HPLC conditions: AS-H column, hexane/ⁱPrOH=98/2, 1.0 mL/min, λ=214 nm, t_{R}(major)=8.2 min, t_{R}(minor)=10.4 min).

### EXAMPLE 54

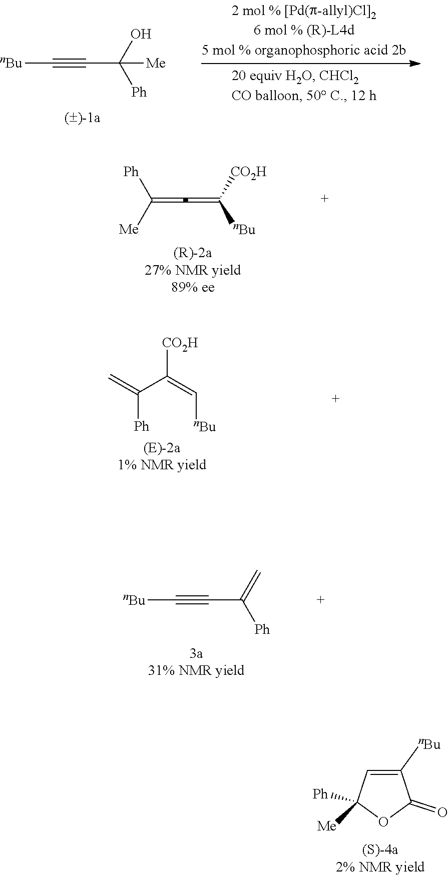

Operations were conducted by referring to Example 1. [Pd(π-allyl)Cl]₂ (0.0015 g, 0.004 mmol), chiral bisphosphine ligand (R)-L4d (0.0147 g, 0.0012 mmol), organophosphoric acid 2b (0.0051 g, 0.01 mmol), (±)-1a (0.0402 g, 0.2 mmol), water (72 μL, d=1.0 g/mL, 0.072 g, 4 mmol), chloroform (1.0 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 27%): 88% ee (HPLC conditions: AS-H column, hexane/ⁱPrOH=98/2, 1.0 mL/min, λ=214 nm, t_{R}(major)=8.5 min, t_{R}(minor)=11.0 min).

### EXAMPLE 55

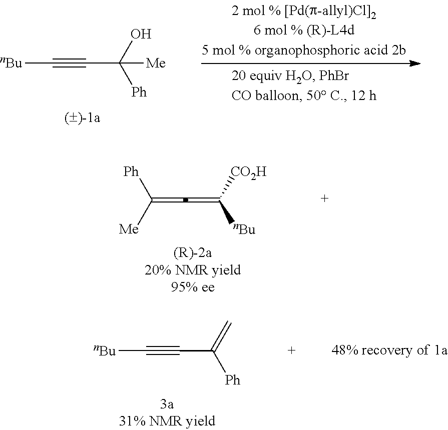

Operations were conducted by referring to Example 1. [Pd(π-allyl)Cl]₂ (0.0016 g, 0.004 mmol), chiral bisphosphine ligand (R)-L4d (0.0146 g, 0.0012 mmol), organophosphoric acid 2a (0.0025 g, 0.01 mmol), (±)-1a (0.0402 g, 0.2 mmol), water (72 d=1.0 g/mL, 0.072 g, 4 mmol), bromobenzene (1.0 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 20%): 95% ee (HPLC conditions: AS-H column, hexane/ⁱPrOH=98/2, 1.0 mL/min, λ=214 nm, t_{R}(major)=7.2 min, t_{R}(minor)=10.9 min).

### EXAMPLE 56

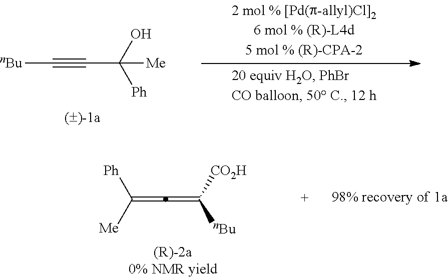

Operations were conducted by referring to Example 1. [Pd(π-allyl)Cl]₂ (0.0016 g, 0.004 mmol), chiral bisphosphine ligand (R)-L4d (0.0147 g, 0.0012 mmol), (R)-CPA-2 (0.0079 g, 0.01 mmol)), (±)-1a (0.0408 g, 0.2 mmol), water (72 μL, d=1.0 g/mL, 0.072 g, 4 mmol), bromobenzene (1.0 mL) were reacted at 50° C. for 12 hours, purified by preparative plate chromatography (developing solvent: petroleum ether (60˜90° C.)/ethyl acetate=5/1) to afford a product: chiral allenoic acid (S)-2a (NMR yield 0%).

### EXAMPLE 57

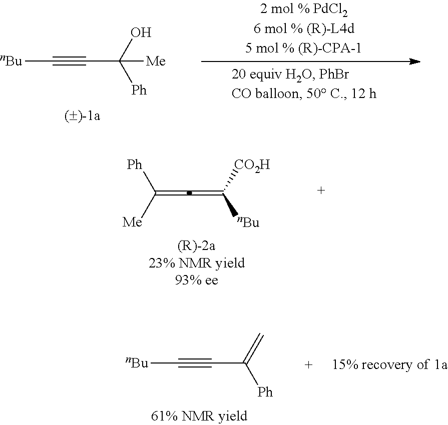

Operations were conducted by referring to Example 1. PdCl₂ (0.0008 g, 0.004 mmol), chiral bisphosphine ligand (R)-L4d (0.0148 g, 0.0012 mmol), (R)-CPA-1 (0.0077 g, 0.01 mmol), (±)-1a (0.0403 g, 0.2 mmol), water (72 μL, d=1.0 g/mL, 0.072 g, 4 mmol), bromobenzene (1.0 mL), were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 23%): 93% ee (HPLC conditions: AS-H column, hexane/ⁱPrOH=98/2, 1.0 mL/min, λ=214 nm, t_{R}(major)=8.8 min, t_{R}(minor)=11.6 min).

### EXAMPLE 58

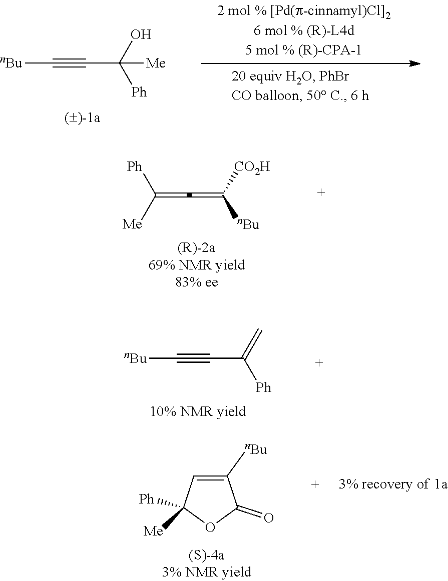

Operations were conducted by referring to Example 1. [Pd(n-cinnamyl)C1]2 (0.0022 g, 0.004 mmol), chiral bisphosphine ligand (R)-L4d (0.0147 g, 0.0012 mmol), (R)-CPA-1 (0.0077 g, 0.01 mmol), (±)-1a (0.0406 g, 0.2 mmol), water (72 μL, d=1.0 g/mL, 0.072 g, 4 mmol), bromobenzene (1.0 mL) were reacted at 50° C. for 6 hours, purified by preparative plate chromatography (developing solvent: petroleum ether (60˜90° C.)/ethyl acetate=5/1) to afford a product: chiral allenoic acid (R)-2a (NMR yield 69%): 83% ee (HPLC conditions: AS-H column, hexane/ⁱPrOH=98/2, 1.0 mL/min, λ=214 nm, t_{R}(major)=8.6 min, t_{R}(minor)=11.5 min).

### EXAMPLE 59

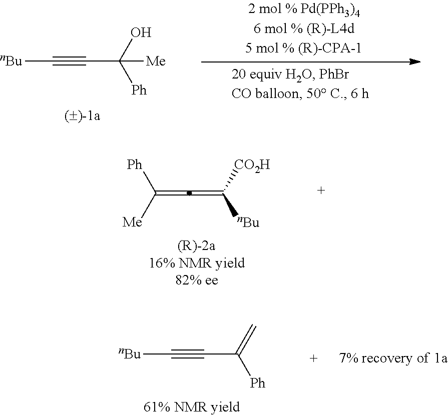

Operations were conducted by referring to Example 1. Pd(PPh₃)4 (0.0046 g, 0.004 mmol), chiral bisphosphine ligand (R)-L4d (0.0147 g, 0.0012 mmol), (R)-CPA-1 (0.0078 g, 0.01 mmol), (±)-1a (0.0407 g, 0.2 mmol), water (72 μL, d=1.0 g/mL, 0.072 g, 4 mmol), bromobenzene (1.0 mL) were reacted at 50° C. for 6 hours, purified by preparative plate chromatography (developing solvent: petroleum ether (60˜90° C.)/ethyl acetate=5/1) to afford a product: chiral allenoic acid (R)-2a (NMR yield 16%): 82% ee (HPLC conditions: AS-H column, hexane/ⁱPrOH=98/2, 1.0 mL/min, λ=214 nm, t_{R}(major)=8.3 min, t_{R}(minor)=11.0 min).

### EXAMPLE 60

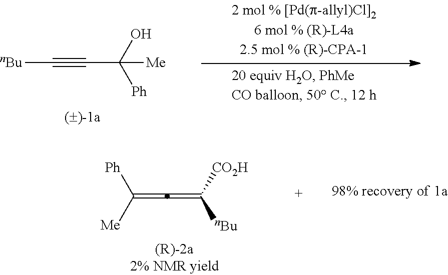

Operations were conducted by referring to Example 1. [Pd(π-allyl)Cl]₂ (0.0015 g, 0.004 mmol), chiral bisphosphine ligand (R)-L4a (0.0078 g, 0.0012 mmol), (R)-CPA-1 (0.0040 g, 0.005 mmol), (±)-1a (0.0402 g, 0.2 mmol), water (72 μL, d=1.0 g/mL, 0.072 g, 4 mmol), toluene (1.0 mL), were reacted at 50° C. for 12 hours. NMR monitored that the reaction hardly occurs.

### EXAMPLE 61

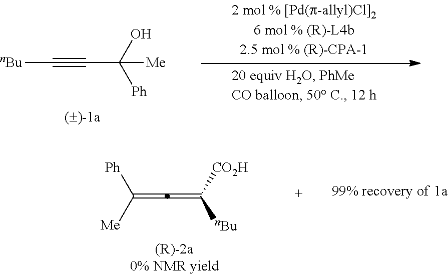

Operations were conducted by referring to Example 1. [Pd(π-allyl)Cl]₂ (0.0015 g, 0.004 mmol), chiral bisphosphine ligand (R)-L4b (0.0087 g, 0.0012 mmol), (R)-CPA-1 (0.0040 g, 0.005 mmol), (±)-1a (0.0405 g, 0.2 mmol), water (72 μL, d=1.0 g/mL, 0.072 g, 4 mmol), toluene (1.0 mL) were reacted at 50° C. for 12 hours. NMR monitored that the reaction did not occur.

### EXAMPLE 62

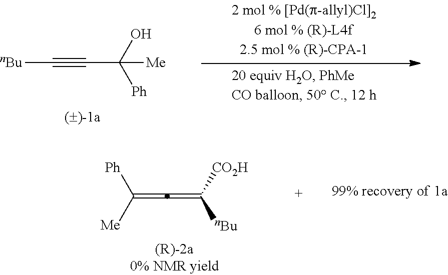

Operations were conducted by referring to Example 1. [Pd(π-allyl)Cl]₂ (0.0016 g, 0.004 mmol), chiral bisphosphine ligand (R)-L4f (0.0151 g, 0.0012 mmol), (R)-CPA-1 (0.0040 g, 0.005 mmol), (±)-1a (0.0404 g, 0.2 mmol), water (72 μL, d=1.0 g/mL, 0.072 g, 4 mmol), toluene (1.0 mL), were reacted at 50° C. for 12 hours. NMR monitored that the reaction did not occur.

### EXAMPLE 63

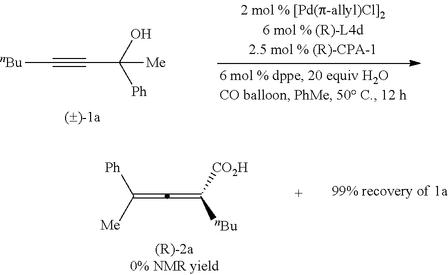

Operations were conducted by referring to Example 1. [Pd(π-allyl)Cl]₂ (0.0015 g, 0.004 mmol), chiral bisphosphine ligand (R)-L4d (0.0146 g, 0.0012 mmol), (R)-CPA-1 (0.0040 g, 0.005 mmol), dppe (0.0049 g, 0.012 mmol), (±)-1a (0.0403 g, 0.2 mmol), water (72 μL, d=1.0 g/mL, 0.072 g, 4 mmol), toluene (1.0 mL) were reacted at 50° C. for 12 hours. NMR monitored that the reaction did not occur.

### EXAMPLE 64

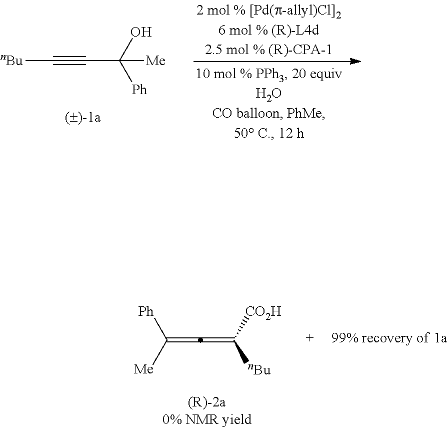

Operations were conducted by referring to Example 1. [Pd(π-allyl)Cl]₂ (0.0015 g, 0.004 mmol), chiral bisphosphine ligand (R)-L4d (0.0145 g, 0.0012 mmol), (R)-CPA-1 (0.0040 g, 0.005 mmol), PPh₃ (0.0053 g, 0.01 mmol), (±)-1a (0.0401 g, 0.2 mmol), water (72 μL, d=1.0 g/mL, 0.072 g, 4 mmol), toluene (1.0 mL) were reacted at 50° C. for 12 hours. NMR monitored that the reaction did not occur.

### EXAMPLE 65

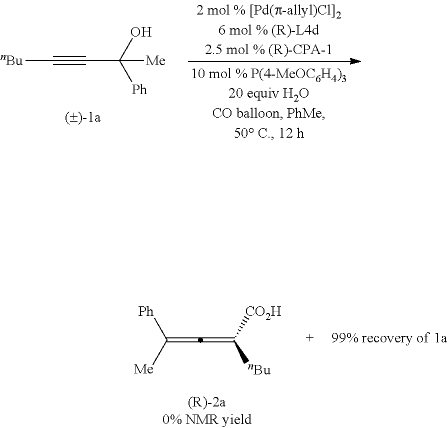

Operations were conducted by referring to Example 1. [Pd(π-allyl)Cl]₂ (0.0014 g, 0.004 mmol), chiral bisphosphine ligand (R)-L4d (0.0146 g, 0.0012 mmol), (R)-CPA-1 (0.0041 g, 0.005 mmol)), P(4-MeOC₆H₄)₃ (0.0069 g, 0.01 mmol), (±)-1a (0.0401 g, 0.2 mmol), water (72 μL, d=1.0 g/mL, 0.072 g, 4 mmol), toluene (1.0 mL) were reacted at 50° C. for 12 hours. NMR monitored that the reaction did not occur.

### EXAMPLE 66

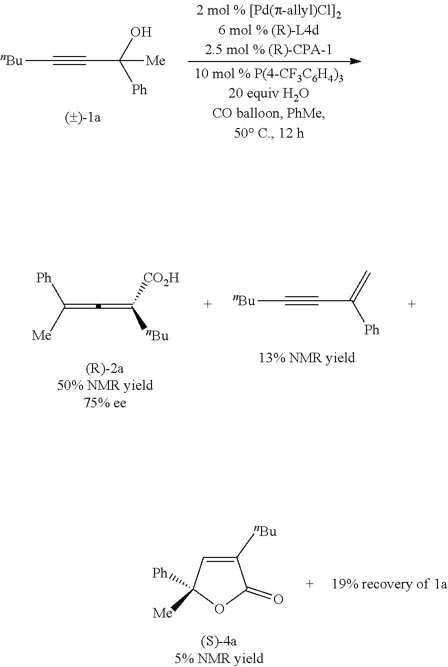

Operations were conducted by referring to Example 1. [Pd(π-allyl)Cl]₂ (0.0015 g, 0.004 mmol), chiral bisphosphine ligand (R)-L4d (0.0144 g, 0.0012 mmol), (R)-CPA-1 (0.0040 g, 0.005 mmol), P(4-CH₃OC₆H₄)₃ (0.0094 g, 0.01 mmol), (±)-1a (0.0398 g, 0.2 mmol), water (72 μL, d=1.0 g/mL, 0.072 g, 4 mmol)), toluene (1.0 mL) reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 50%): 75% ee (HPLC conditions: AS-H column, hexane/ⁱPrOH=98/2, 1.0 mL/min, λ=214 nm, t_{R}(major)=8.9 min, t_{R}(minor)=12.4 min).

### EXAMPLE 67

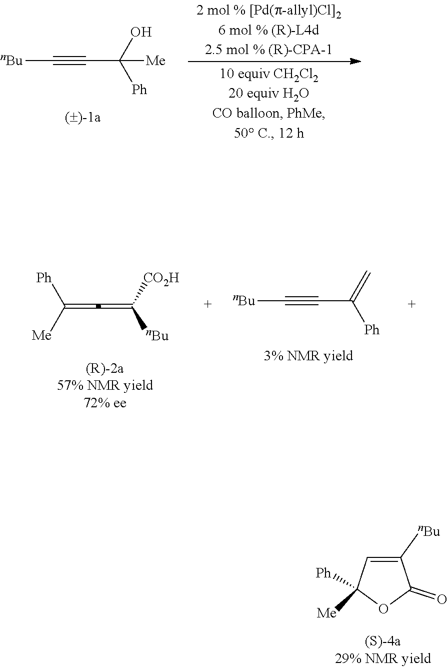

Operations were conducted by referring to Example 1. [Pd(π-allyl)Cl]₂ (0.0015 g, 0.004 mmol), chiral bisphosphine ligand (R)-L4d (0.0145 g, 0.0012 mmol), (R)-CPA-1 (0.0040 g, 0.005 mmol), CH₂Cl₂ (128 μL, d=1.32 g/mL, 0.1698 g, 2 mmol), (±)-1a (0.0400 g, 0.2 mmol), water (72 μL, d=1.0 g/mL, 0.072 g, 4 mmol), toluene (0.88 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 57%): 72% ee (HPLC conditions: AS-H column, hexane/ⁱPrOH=98/2, 1.0 mL/min, λ=214 nm, t_{R}(major)=8.9 min, t_{R}(minor)=12.1 min).

### EXAMPLE 68

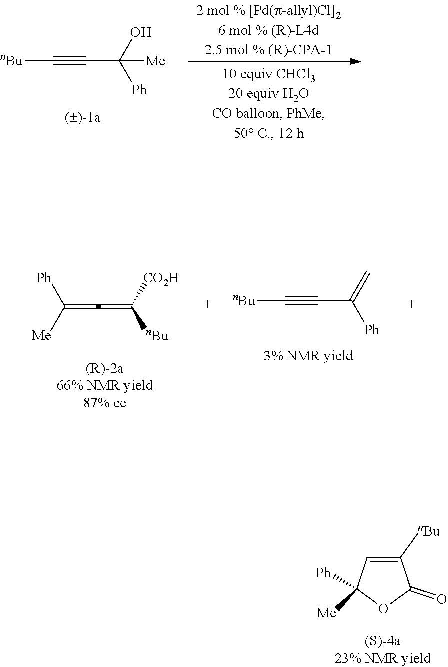

Operations were conducted by referring to Example 1. [Pd(π-allyl)Cl]₂ (0.0015 g, 0.004 mmol), chiral bisphosphine ligand (R)-L4d (0.0146 g, 0.0012 mmol), (R)-CPA-1 (0.0041 g, 0.005 mmol), CHCl₃ (161 μL, d=1.48 g/mL, 0.2388 g, 2 mmol), (±)-1a (0.0399 g, 0.2 mmol), water (72 μL, d=1.0 g/mL, 0.072 g, 4 mmol), toluene (0.84 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 66%): 87% ee (HPLC conditions: AS-H column, hexane/ⁱPrOH=98/2, 1.0 mL/min, λ=214 nm, t_{R}(major)=9.0 min, t_{R}(minor)=12.3 min).

### EXAMPLE 69

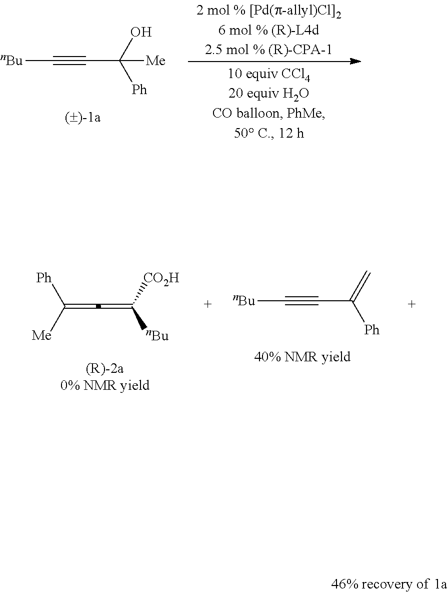

Operations were conducted by referring to Example 1. [Pd(π-allyl)Cl]₂ (0.0016 g, 0.004 mmol), chiral bisphosphine ligand (R)-L4d (0.0146 g, 0.0012 mmol), (R)-CPA-1 (0.0040 g, 0.005 mmol)), CCl₄ (193 μL, d=1.02 g/mL, 0.3076 g, 2 mmol), (±)-1a (0.0401 g, 0.2 mmol), water (72 μL, d=1.0 g/mL, 0.072 g, 4 mmol), toluene (0.81 mL) were reacted at 50° C. for 12 hours. NMR monitored that the reaction did not occur.

### EXAMPLE 70

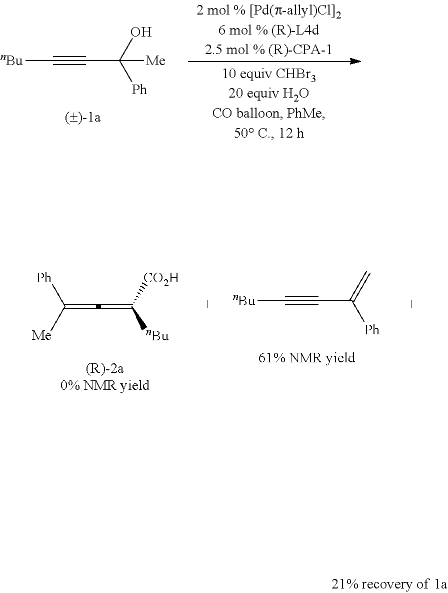

Operations were conducted by referring to Example 1. [Pd(π-allyl)Cl]₂ (0.0014 g, 0.004 mmol), chiral bisphosphine ligand (R)-L4d (0.0146 g, 0.0012 mmol), (R)-CPA-1 (0.0040 g, 0.005 mmol), CHBr₃ (174 μL, d=2.89 g/mL, 0.502 g, 2 mmol), (±)-1a (0.0401 g, 0.2 mmol), water (72 μL, d=1.0 g/mL, 0.072 g, 4 mmol), toluene (0.83 mL) were reacted at 50° C. for 12 hours. NMR monitored that the reaction did not occur.

### EXAMPLE 71

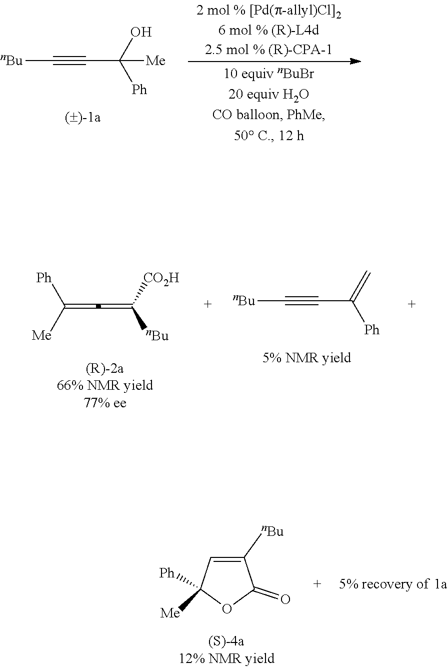

Operations were conducted by referring to Example 1. [Pd(π-allyl)Cl]₂ (0.0016 g, 0.004 mmol), chiral bisphosphine ligand (R)-L4d (0.0145 g, 0.0012 mmol), (R)-CPA-1 (0.0040 g, 0.005 mmol), ⁿBuBr (214 μL, d=1.28 g/mL, 0.274 g, 2 mmol), (±)-1a (0.0400 g, 0.2 mmol), water (72 μL, d=1.0 g/mL, 0.072 g, 4 mmol), toluene (0.8 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 66%): 77% ee (HPLC conditions: AS-H column, hexane/ⁱPrOH=98/2, 1.0 mL/min, λ=214 nm, t_{R}(major)=9.0 min, t_{R}(minor)=12.6 min).

### EXAMPLE 72

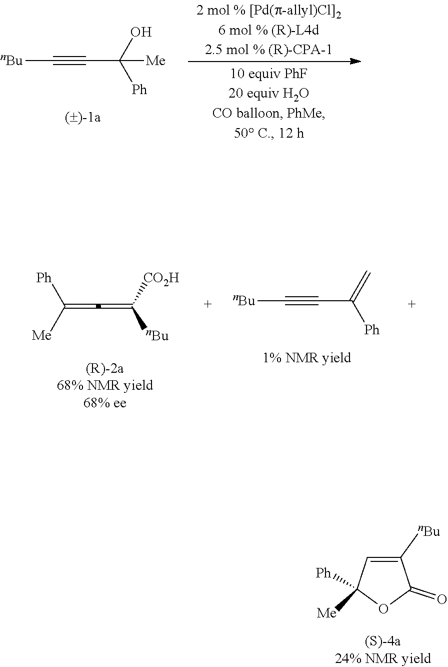

Operations were conducted by referring to Example 1. [Pd(π-allyl)Cl]₂ (0.0016 g, 0.004 mmol), chiral bisphosphine ligand (R)-L4d (0.0145 g, 0.0012 mmol), (R)-CPA-1 (0.0041 g, 0.005 mmol), PhF (188 μL, d=1.02 g/mL, 0.1922 g, 2 mmol), (±)-1a (0.0403 g, 0.2 mmol), water (72 μL, d=1.0 g/mL, 0.072 g, 4 mmol), toluene (0.81 mL) were reacted at 50° C. for 12 hours, purifiec by preparative plate chromatography (developing solvent: petroleum ether (60˜90° C.)/ethyl acetate=5/1) to afford a product: chiral allenoic acid (R)-2a (NMR yield 68%): 68% ee (HPLC conditions: AS-H column, hexane/ⁱPrOH=98/2, 1.0 mL/min, λ=214 nm, t_{R}(major)=9.3 min, t_{R}(minor)=13.2 min).

### EXAMPLE 73

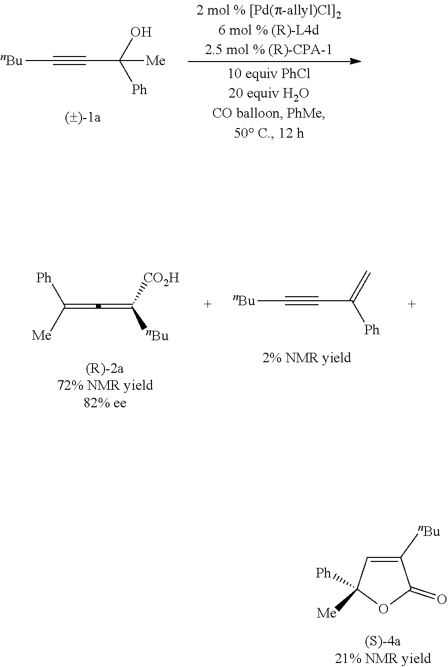

Operations were conducted by referring to Example 1. [Pd(π-allyl)Cl]₂ (0.0015 g, 0.004 mmol), chiral bisphosphine ligand (R)-L4d (0.0147 g, 0.0012 mmol), (R)-CPA-1 (0.0041 g, 0.005 mmol), PhCl (220 μL, d=1.02 g/mL, 0.2252 g, 2 mmol), (±)-1a (0.0400 g, 0.2 mmol), water (72 μL, d=1.0 g/mL, 0.072 g, 4 mmol), toluene (0.78 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 72%): 82% ee (HPLC conditions: AS-H column, hexane/ⁱPrOH=98/2, 1.0 mL/min, λ=214 nm, t_{R}(major)=9.3 min, t_{R}(minor)=12.8 min).

### EXAMPLE 74

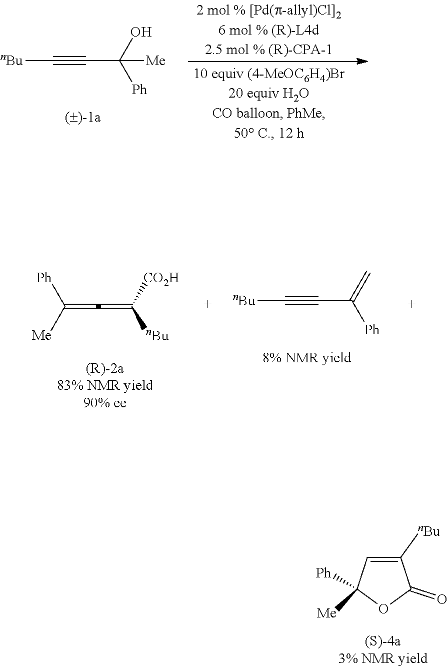

Operations were conducted by referring to Example 1. [Pd(π-allyl)Cl]₂ (0.0015 g, 0.004 mmol), chiral bisphosphine ligand (R)-L4d (0.0145 g, 0.0012 mmol), (R)-CPA-1 (0.0040 g, 0.005 mmol), (4-MeOC₆H₄)Br (250 μL, d=1.49 g/mL, 0.374 g, 2 mmol), (±)-1a (0.0405 g, 0.2 mmol), water (72 μL, d=1.0 g/mL, 0.072 g, 4 mmol), toluene (0.75 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 83%): 90% ee (HPLC conditions: AS-H column, hexane/ⁱPrOH=98/2, 1.0 mL/min, λ=214 nm, t_{R}(major)=9.3 min, t_{R}(minor)=13.4 min).

### EXAMPLE 75

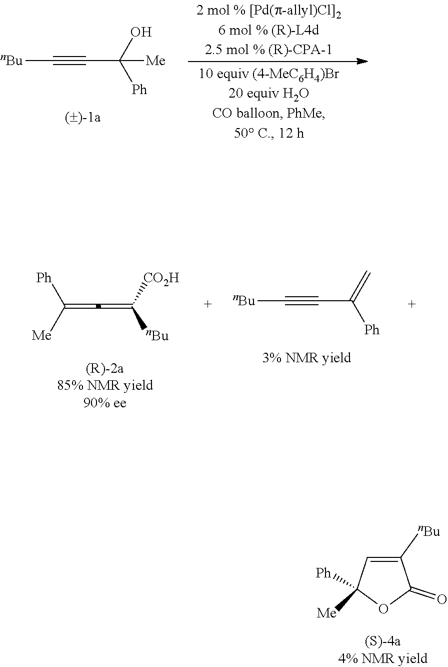

Operations were conducted by referring to Example 1. [Pd(π-allyl)Cl]₂ (0.0016 g, 0.004 mmol), chiral bisphosphine ligand (R)-L4d (0.0146 g, 0.0012 mmol), (R)-CPA-1 (0.0040 g, 0.005 mmol), (4-MeC₆H₄)Br (220 μL, d=1.55 g/mL, 0.342 g, 2 mmol), (±)-1a (0.0405 g, 0.2 mmol), water (72 μL, d=1.0 g/mL), 0.072 g, 4 mmol), toluene (0.78 mL) were reacted at 50° C. for 12 hours, purified by preparative plate chromatography (developing solvent: petroleum ether (60˜90° C.)/ethyl acetate=5/1) to afford a product: chiral allenoic acid (R)-2a (NMR yield 85%): 90% ee (HPLC conditions: AS-H column, hexane/ⁱPrOH=98/2, 1.0 mL/min, λ=214 nm, t_{R}(major)=9.4 min, t_{R}(minor)=13.5 min).

### EXAMPLE 76

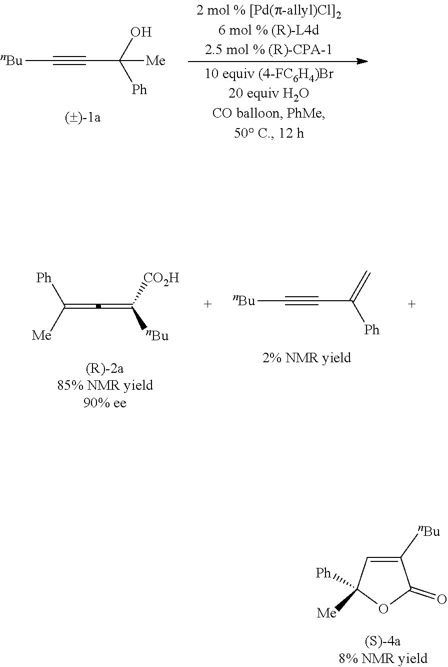

Operations were conducted by referring to Example 1. [Pd(π-allyl)Cl]₂ (0.0015 g, 0.004 mmol), chiral bisphosphine ligand (R)-L4d (0.0148 g, 0.0012 mmol), (R)-CPA-1 (0.0040 g, 0.005 mmol), (4-FC₆H₄)Br (220 μL, d=1.59 g/mL, 0.350 g, 2 mmol), (±)-1a (0.0405 g, 0.2 mmol), water (72 μL, d=1.0 g/mL), 0.072 g, 4 mmol), toluene (0.78 mL) were reacted at 50° C. for 12 hours, purified by preparative plate chromatography (developing solvent: petroleum ether (60˜90° C.)/ethyl acetate=5/1) to afford a product: chiral allenoic acid product (R)-2a (NMR yield 85%): 90% ee (HPLC conditions: AS-H column, hexane/ⁱPrOH=98/2, 1.0 mL/min, λ=214 nm, t R t_{R}(major)=9.3 min, t_{R}(minor)=13.4 min).

### EXAMPLE 77

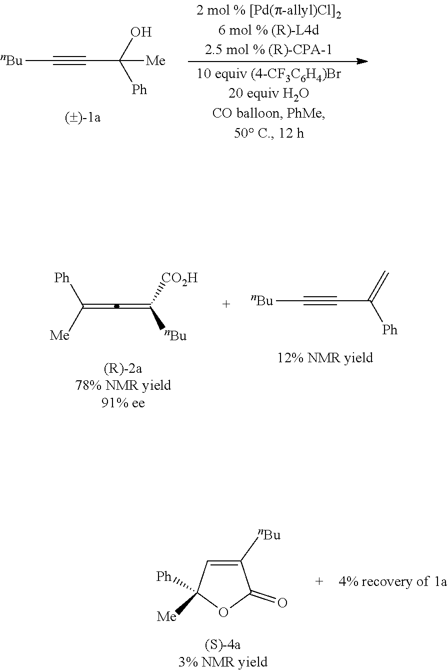

Operations were conducted by referring to Example 1. [Pd(π-allyl)Cl]₂ (0.0015 g, 0.004 mmol), chiral bisphosphine ligand (R)-L4d (0.0146 g, 0.0012 mmol), (R)-CPA-1 (0.0040 g, 0.005 mmol)), (4-CF₃C₆H₄)Br (280 μL, d=1.61 g/mL, 0.450 g, 2 mmol), (±)-1a (0.0401 g, 0.2 mmol), water (72 μL, d=1.0 g/mL, 0.072 g, 4 mmol), toluene (0.78 mL) were reacted at 50° C. for 12 hours, purified by preparative plate chromatography (developing solvent: petroleum ether (60˜90° C.)/ethyl acetate=5/1) to afford a product: chiral allenoic acid product (R)-2a (NMR yield: 78%): 91% ee (HPLC conditions: AS-H column, hexane/ⁱPrOH=98/2, 1.0 mL/min, λ=214 nm, t_{R}(major)=9.3 min, t_{R}(minor)=13.5 min).

Ordinary technicians in this field will understand that within the protection scope of the invention, it is feasible to modify, add and replace the above implementation cases, and none of them is beyond the protection scope of the invention.

1. A method for preparing a chiral tetra-substituted allenoic acid compound based on a palladium catalytic system, wherein, in the presence of palladium catalyst, chiral bisphosphine ligand, organophosphoric acid and organic additive, the tertiary propargyl alcohol with different substituents, carbon monoxide and water undergo the asymmetric allenylation reaction in an organic solvent through transition metal catalysis, constructing highly optically active axially chiral allenoic acid compound in one-step synthesis, the reaction process has the following reaction equation (a): 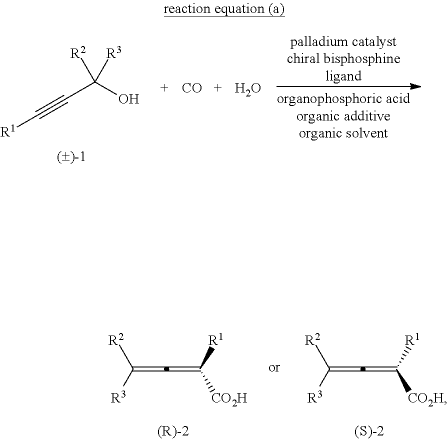
wherein, R¹ is an alkyl, an alkyl with functional group, phenyl, aryl or heterocyclic group; R² is an alkyl, an alkyl with functional group, phenyl, aryl or heterocyclic group; R³ is an alkyl, an alkyl with functional group, phenyl, aryl or heterocyclic group;
in R¹, R² and R³, said functional group is selected from carbon-carbon triple bond, hydroxyl, acyl, acyloxy, amide, amino, and silicon group; said aryl group is phenyl group with electron-donating or electron-withdrawing substituents at the ortho, meta, and para positions; said heterocyclyl group is furyl or pyridyl group, or furan or pyridine with electron-donating or electron-withdrawing substituents.

2. The method of claim 1, wherein, R¹ is a C1-C30 alkyl, a C1-C30 alkyl with functional group at the end, phenyl, aryl or heterocyclic group; R² is a C1-C10 alkyl, a C1-C10 alkyl with functional group at the end, phenyl, aryl or heterocyclic group; R³ is C1-C10 alkyl, a C1-C10 alkyl with functional group at the end, phenyl, aryl or heterocyclic group;
in R¹, R² and R³, in said C1-C30 alkyl with functional group at the end or said C1-C10 alkyl with functional group at the end, said functional group is selected from carbon-carbon triple bond, hydroxyl, acyl, acyloxy, amide, amino, silicon group; said aryl group is phenyl group with electron-donating or electron-withdrawing substituents at the ortho, meta and para positions, said heterocyclic group is a furanyl or pyridyl group, or furan or pyridine with electron-donating or electron-withdrawing substituents; said electron-withdrawing substituents in the aryl or heterocyclic group include halogen, nitro, ester, carboxyl, acyl, amide, and cyano group, and said electron-donating substituents include alkyl, alkenyl, phenyl, alkoxy group, hydroxyl, amino, silicon group.

3. The method of claim 1, wherein, said method comprises the following steps:
1) adding a palladium catalyst, a chiral bisphosphine ligand and an organophosphoric acid in sequence into a dried reaction tube, plugging the reaction tube with a rubber stopper, connecting the vacuum pump, replacing with argon under argon atmosphere, adding a functionalized tertiary propargyl alcohol, water, organic additives, and a certain volume of organic solvent; freezing the reaction tube in liquid nitrogen bath, inserting carbon monoxide balloon, replacing with carbon monoxide into the reaction system under the atmosphere of carbon monoxide; after freezing and pumping, when the reaction system returns to the room temperature and melts, putting the reaction tube in the preset low-temperature bath at −20˜80° C. or oil bath and stirring for 4-36 hours; wherein, said organic solvent with a certain volume refers to the amount of functionalized tertiary propargyl alcohol shown in equation (a) as a basis, and said dosage of the organic solvent is 1.0-10.0 mL/mmol;
2) after the completion of the reaction in step 1), raising the reaction tube from the oil bath, after returning to the room temperature, adding a certain volume of ethyl acetate into the reaction tube, filtering the resulting mixture with silica gel short column, washing with a certain amount of ethyl acetate, concentrating, and subjecting to the flash column chromatography, so as to obtain the highly optically active axially chiral allenoic acid compounds; wherein, the certain volume of the ethyl acetate refers to the amount of functionalized tertiary propargyl alcohol shown in equation (a) as a basis, said amount of ethyl acetate is 1.0-100 mL/mmol.

4. The method of claim 1, wherein, said palladium catalysts are any one or more of dis-(allyl-palladium chloride), tetra-(triphenylphosphine)palladium, tri-(dibenzylidene-acetone)dipalladium, dis-(cinnamyl-palladium chloride), dis-(dibenzylidene-acetone)monopalladium, palladium chloride, palladium acetate, dis-(triphenylphosphine)palladium chloride and bis-(acetonitrile)palladium chloride.

5. The method of claim 1, wherein, said chiral diphosphine ligand is selected from one or more of (R)-L1˜(R)-L4 and its enantiomer (S)-L1˜(S)-L4 in the following structures; wherein, Ar is a phenyl, an aryl or heterocyclic group;
said aryl group is a phenyl group substituted by alkyl or alkoxy group at the ortho, meta, and para positions; said heterocyclic group is thiophene, furan, or pyridine and thiophene substituted by alkyl or alkoxy group, furan substituted by alkyl or alkoxy group, or pyridine substituted by alkyl or alkoxy group:

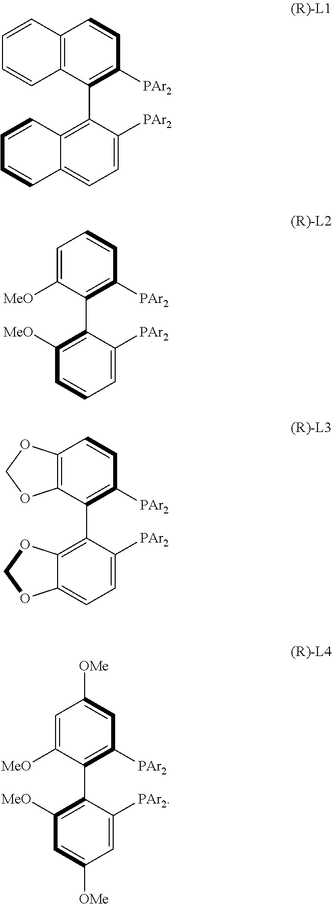

6. The method of claim 5, wherein said chiral diphosphine ligand is selected from (R)-L4 and its enantiomer (S)-L4, the said structure of (R)-L4 is as follows:
Wherein, Ar is 3,5-dialkyl-4-alkoxyphenyl, 3,5-dialkylphenyl, 4-alkylphenyl or phenyl group;

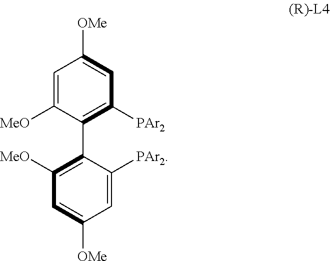

7. The method of claim 1, wherein said organophosphoric acid is selected from any one or more of organophosphoric acid 1, organophosphoric acid 2, organophosphoric acid 3, the structure of which is as follows; wherein, R⁴ is C1˜C6 alkyl, phenyl or aryl group; said aryl group is a phenyl group substituted by C1˜C6 alkyl at the ortho, meta, and para positions; R⁵ is hydrogen, C1˜C6 alkyl, phenyl or aryl group;
said aryl group is a phenyl group substituted by C1˜C6 alkyl at the ortho, meta, and para positions;

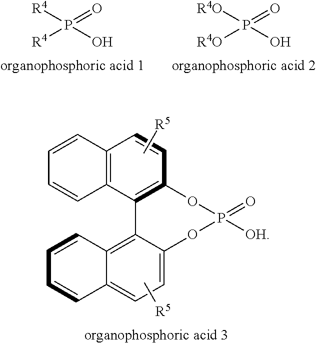

8. The method of claim 1, wherein, said organic additive is selected from any one or more of 1,1-bis(diphenylphosphine)methane, 1,2-bis(diphenylphosphine)ethane, 1,3-bis(diphenylphosphine)propane, 1,4-bis(diphenylphosphine)butane, 1,1′-bis(diphenylphosphine)ferrocene, bis(2-diphenylphosphine)ether, 4,5-bis(diphenylphosphino)-9,9-dimethylxanthene, 1,1′-binaphthyl-2,2′-bisdiphenylphosphine, triphenylphosphine, tri(4-methoxyphenyl)phosphine, tri(4-methylphenyl)phosphine, tri(4-fluorophenyl)phosphine, tris(4-trifluoromethylphenyl)phosphine, dichloromethane, dibromomethane, chloroform, bromoform, carbon tetrachloride, bromoethane, bromobutane, benzene, fluorobenzene, 1,4-difluorobenzene, hexafluorobenzene, chlorobenzene, 1,4-dichlorobenzene, bromobenzene, 1,4-dibromobenzene, 4-methoxybromobenzene, 4-methylbromobenzene, 4-fluorobromobenzene, 4-trifluoromethylbromobenzene, iodobenzene, trifluorotoluene, aniline, benzenesulfonic acid, phenol, phenylboronic acid; and/or, said organic solvent is selected from any one or more of N-methyl pyrrolidone, 1,4-dioxane, tetrahydrofuran, acetonitrile, methyl tert-butyl ether, fluorobenzene, chlorobenzene, bromobenzene, iodobenzene, toluene, 1,2-xylene, 1,3-xylene, 1,4-xylene, mesitylene, 4-ethyltoluene, 1,4-diethylbenzene, triethylbenzene, trifluorotoluene, dichloromethane, dibromomethane, 1,1-dichloroethane, 1,2-dichloroethane, 1,2-dibromoethane, chloroform, acetic acid, N,N-dimethylformamide and dimethyl sulfoxide.

9. The method of claim 1, wherein, the molar ratio of tertiary propargyl alcohol (±1) with different substituents, water, palladium catalyst, chiral bisphosphine ligand, organophosphoric acid, and organic additives is 1.0:(1.0-30.0):(0.005-0.1):(0.005-0.1):(0.01-0.3):(0.1-30); and/or, the reaction temperature is −20 —100° C.; and/or, the dosage of the organic solvent is 1.0-10.0 mL/mmol, based on the dosage of functionalized tertiary propargyl alcohol (±1).

10. A class of highly optically active axially chiral allenoic acid compound, wherein, the structure is as (R)-2, (S)-2 shown: 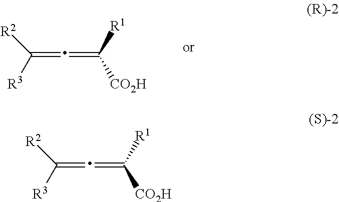
wherein, R¹ is an alkyl, an alkyl with functional group, phenyl, aryl or heterocyclic group; R² is an alkyl, an alkyl with functional group, phenyl, aryl or heterocyclic group; R³ is an alkyl, an alkyl with functional group, phenyl, aryl or heterocyclic groups;
in R¹, R² and R³, said functional group is selected from carbon-carbon triple bond, hydroxyl, acyl, acyloxy, amide, amino, and silicon group; said aryl group is phenyl group with electron-donating or electron-withdrawing substituents at the ortho, meta, and para positions; said heterocyclyl group is furyl or pyridyl group, or furan or pyridine with electron-donating or electron-withdrawing substituents.

11. The compound of claim 10, wherein, R¹ is a C1-C30 alkyl, a C1-C30 alkyl with functional group at the end, phenyl, aryl or heterocyclic group; R² is a C1-C10 alkyl, a C1-C10 alkyl with functional group at the end, phenyl, aryl or heterocyclic group; R³ is C1-C10 alkyl, a C1-C10 alkyl with functional group at the end, phenyl, aryl or heterocyclic group;
in R¹, R² and R³, in the C1-C30 alkyl with functional group at the end or the C1-C10 alkyl with functional group at the end, the functional group is selected from carbon-carbon triple bond, hydroxyl, acyl, acyloxy, amide, amino, silicon group; said aryl group is phenyl group with electron-donating or electron-withdrawing substituents at the ortho, meta and para positions, said heterocyclic group is a furanyl or pyridyl group, or furan or pyridine with electron-donating or electron-withdrawing substituents; said electron-withdrawing substituents in the aryl or heterocyclic group include halogen, nitro, ester, carboxyl, acyl, amide, and cyano groups, and the electron-donating substituents include alkyl, alkenyl, phenyl, alkoxy group, hydroxyl, amino, silicon group.

12. A highly optically active axially chiral allenoic acid compound according to claim 10 for use to preparing γ-butyrolactone compound containing a tetra-substituted chiral quaternary carbon center, tetra-substituted allenol, tetra-substituted allenal, tetra-substituted allenyl ketone, tetra-substituted allenamide compounds.

13. The method of claim 2, wherein, said palladium catalysts are any one or more of dis-(allyl-palladium chloride), tetra-(triphenylphosphine)palladium, tri-(dibenzylidene-acetone)dipalladium, dis-(cinnamyl-palladium chloride), dis-(dibenzylidene-acetone)monopalladium, palladium chloride, palladium acetate, dis-(triphenylphosphine)palladium chloride and bis-(acetonitrile)palladium chloride.

14. The method of claim 3, wherein, said palladium catalysts are any one or more of dis-(allyl-palladium chloride), tetra-(triphenylphosphine)palladium, tri-(dibenzylidene-acetone)dipalladium, dis-(cinnamyl-palladium chloride), dis-(dibenzylidene-acetone)monopalladium, palladium chloride, palladium acetate, dis-(triphenylphosphine)palladium chloride and bis-(acetonitrile)palladium chloride.

15. The method of claim 2, wherein, said chiral diphosphine ligand is selected from one or more of (R)-L1˜(R)-L4 and its enantiomer (S)-L1—(S)-L4 in the following structures; wherein, Ar is a phenyl, an aryl or heterocyclic group; said aryl group is a phenyl group substituted by alkyl or alkoxy group at the ortho, meta, and para positions; said heterocyclic group is thiophene, furan, or pyridine and thiophene substituted by alkyl or alkoxy group, furan substituted by alkyl or alkoxy group, or pyridine substituted by alkyl or alkoxy group;

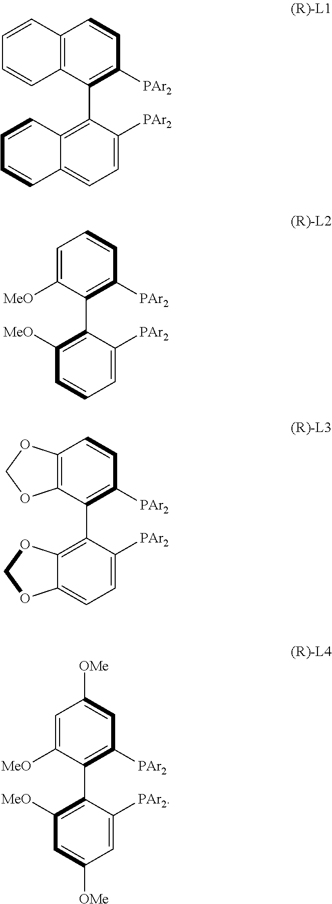

16. The method of claim 3, wherein, said chiral diphosphine ligand is selected from one or more of (R)-L1˜(R)-L4 and its enantiomer (S)-L1˜(S)-L4 in the following structures; wherein, Ar is a phenyl, an aryl or heterocyclic group; said aryl group is a phenyl group substituted by alkyl or alkoxy group at the ortho, meta, and para positions; said heterocyclic group is thiophene, furan, or pyridine and thiophene substituted by alkyl or alkoxy group, furan substituted by alkyl or alkoxy group, or pyridine substituted by alkyl or alkoxy group;

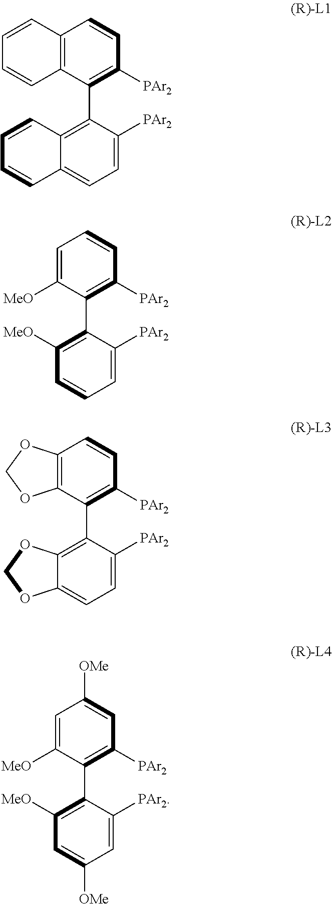

17. The method of claim 2, wherein said organophosphoric acid is selected from any one or more of organophosphoric acid 1, organophosphoric acid 2, organophosphoric acid 3, the structure of which is as follows; wherein, R⁴ is C1˜C6 alkyl, phenyl or aryl group; said aryl group is a phenyl group substituted by C1˜C6 alkyl at the ortho, meta, and para positions; R⁵ is hydrogen, C1˜C6 alkyl, phenyl or aryl group; said aryl group is a phenyl group substituted by C1˜C6 alkyl at the ortho, meta, and para positions;

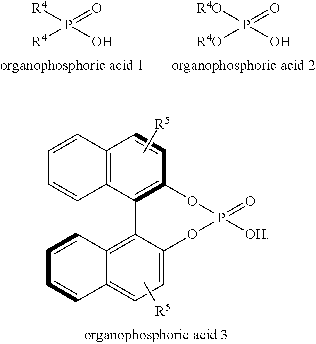

18. The method of claim 3, wherein said organophosphoric acid is selected from any one or more of organophosphoric acid 1, organophosphoric acid 2, organophosphoric acid 3, the structure of which is as follows; wherein, R⁴ is C1˜C6 alkyl, phenyl or aryl group; said aryl group is a phenyl group substituted by C1˜C6 alkyl at the ortho, meta, and para positions; R⁵ is hydrogen, C1˜C6 alkyl, phenyl or aryl group; said aryl group is a phenyl group substituted by C1˜C6 alkyl at the ortho, meta, and para positions;

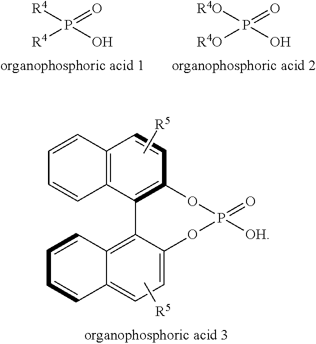

19. The method of claim 2, wherein, said organic additive is selected from any one or more of 1,1-bis(diphenylphosphine)methane, 1,2-bis(diphenylphosphine)ethane, 1,3-bis(diphenylphosphine)propane, 1,4-bis(diphenylphosphine)butane, 1,1′-bis(diphenylphosphine)ferrocene, bis(2-diphenylphosphine)ether, 4,5-bis(diphenylphosphino)-9,9-dimethylxanthene, 1,1′-binaphthyl-2,2′-bisdiphenylphosphine, triphenylphosphine, tri(4-methoxyphenyl)phosphine, tri(4-methylphenyl)phosphine, tri(4-fluorophenyl)phosphine, tris(4-trifluoromethylphenyl)phosphine, dichloromethane, dibromomethane, chloroform, bromoform, carbon tetrachloride, bromoethane, bromobutane, benzene, fluorobenzene, 1,4-difluorobenzene, hexafluorobenzene, chlorobenzene, 1,4-dichlorobenzene, bromobenzene, 1,4-dibromobenzene, 4-methoxybromobenzene, 4-methylbromobenzene, 4-fluorobromobenzene, 4-trifluoromethylbromobenzene, iodobenzene, trifluorotoluene, aniline, benzenesulfonic acid, phenol, phenylboronic acid; and/or, said organic solvent is selected from any one or more of N-methyl pyrrolidone, 1,4-dioxane, tetrahydrofuran, acetonitrile, methyl tert-butyl ether, fluorobenzene, chlorobenzene, bromobenzene, iodobenzene, toluene, 1,2-xylene, 1,3-xylene, 1,4-xylene, mesitylene, 4-ethyltoluene, 1,4-diethylbenzene, triethylbenzene, trifluorotoluene, dichloromethane, dibromomethane, 1,1-dichloroethane, 1,2-dichloroethane, 1,2-dibromoethane, chloroform, acetic acid, N,N-dimethylformamide and dimethyl sulfoxide.

20. The method of claim 3, wherein, said organic additive is selected from any one or more of 1,1-bis(diphenylphosphine)methane, 1,2-bis(diphenylphosphine)ethane, 1,3-bis(diphenylphosphine)propane, 1,4-bis(diphenylphosphine)butane, 1,1′-bis(diphenylphosphine)ferrocene, bis(2-diphenylphosphine)ether, 4,5-bis(diphenylphosphino)-9,9-dimethylxanthene, 1,1′-binaphthyl-2,2′-bisdiphenylphosphine, triphenylphosphine, tri(4-methoxyphenyl)phosphine, tri(4-methylphenyl)phosphine, tri(4-fluorophenyl)phosphine, tris(4-trifluoromethylphenyl)phosphine, dichloromethane, dibromomethane, chloroform, bromoform, carbon tetrachloride, bromoethane, bromobutane, benzene, fluorobenzene, 1,4-difluorobenzene, hexafluorobenzene, chlorobenzene, 1,4-dichlorobenzene, bromobenzene, 1,4-dibromobenzene, 4-methoxybromobenzene, 4-methylbromobenzene, 4-fluorobromobenzene, 4-trifluoromethylbromobenzene, iodobenzene, trifluorotoluene, aniline, benzenesulfonic acid, phenol, phenylboronic acid; and/or, said organic solvent is selected from any one or more of N-methyl pyrrolidone, 1,4-dioxane, tetrahydrofuran, acetonitrile, methyl tert-butyl ether, fluorobenzene, chlorobenzene, bromobenzene, iodobenzene, toluene, 1,2-xylene, 1,3-xylene, 1,4-xylene, mesitylene, 4-ethyltoluene, 1,4-diethylbenzene, triethylbenzene, trifluorotoluene, dichloromethane, dibromomethane, 1,1-dichloroethane, 1,2-dichloroethane, 1,2-dibromoethane, chloroform, acetic acid, N,N-dimethylformamide and dimethyl sulfoxide.
